(19) United States
(12) Patent Application Publication
(10) Pub. No.: US 2013/0116304 A1
(43) Pub. Date: May 9, 2013

(54) TMEM195 ENCODES FOR TETRAHYDROBIOPTERIN-DEPENDENT ALKYLGLYCEROL MONOOXYGENASE ACTIVITY

(71) Applicants: Ernst R. Werner, Innsbruck (AT); Katrin Watschinger, Innsbruck (AT)

(72) Inventors: Ernst R. Werner, Innsbruck (AT); Katrin Watschinger, Innsbruck (AT)

(73) Assignee: Ernst R. Werner, Innsbruck (AT)

(21) Appl. No.: 13/641,913

(22) PCT Filed: Apr. 18, 2011
(86) PCT No.: PCT/EP2011/056142

§ 371 Date: Jan. 7, 2013

(30) Foreign Application Priority Data
Apr. 19, 2010 (EP) 10160368.6

(51) Int. Cl. C12N 9/00

(52) U.S. Cl. CPC C12N 9/0071
USPC 514/44 A; 435/320.1; 435/358; 435/189; 435/7.1; 435/6.11; 536/23.2; 530/387.9; 536/24.5; 514/44 R

(57) ABSTRACT

The present invention relates to the provision of a pharmaceutical composition comprising a nucleic acid molecule encoding a alkylglycerol monooxygenase (TMEM195; glyceryl ether monooxygenase; EC 1.14.16.5). The present invention also provides for a method for producing said alkylglycerol monooxygenase (TMEM195; glyceryl ether monooxygenase; EC 1.14.16.5) polypeptides encoded by said polynucleotides. Moreover, the use of such polypeptides as well as of antagonists/inhibitors of such polypeptides in a medical setting (e.g. in from of a pharmaceutical composition) and methods for assessing the activity of a candidate molecule suspected of being an antagonist/inhibitor or agonist/activator in order to identify potential antagonists/inhibitors or agonists/activators of the polypeptide are also provided in the present invention. Finally, the present invention provides kits for carrying out said methods wherein the kits comprise polynucleotides and/or antibodies capable of detecting the activity of alkylglycerol monooxygenase.

The present invention relates to the provision of a pharmaceutical composition comprising a nucleic acid molecule encoding a alkylglycerol monooxygenase (TMEM195; glyceryl ether monooxygenase; EC 1.14.16.5) comprising a polynucleotide selected from the group consisting of: (a) a polynucleotide sequence as shown in SEQ ID NO:1 or a fragment thereof; (b) a polynucleotide sequence encoding a polypeptide as shown in SEQ ID NO:2 or a fragment thereof; (c) a polynucleotide sequence which has at least 80% identity to the polynucleotides as defined in (a) or (b) encoding a functional alkylglycerol monooxygenase or a fragment thereof; (d) a polynucleotide sequence which hybridizes to the polynucleotide sequence of any one of (a) to (c) and whereby the coding strand encodes a functional alkylglycerol monooxygenase or a fragment thereof; (e) a polynucleotide sequence encoding a polypeptide as encoded by the nucleotide sequence of any one of (a) to (d) wherein at least one amino acid is deleted, substituted, inserted or added and whereby said polynucleotide encodes a alkylglycerol monooxygenase or a fragment thereof; (f) a polynucleotide sequence being degenerate as a result of the genetic code to the nucleotide sequence as defined in any one of (a) to (e); and (g) the complementary strand of the polynucleotide of any one of (a) to (f). The present invention also provides for a method for producing said alkylglycerol monooxygenase (TMEM195; glyceryl ether monooxygenase; EC 1.14.16.5) polypeptides encoded by said polynucleotides. Moreover, the use of such polypeptides as well as of antagonists/inhibitors of such polypeptides in a medical setting (e.g. in form of a pharmaceutical composition) and methods for assessing the activity of a candidate molecule suspected of being an antagonist/inhibitor or agonist/activator in order to identify potential antagonists/inhibitors or agonists/activators of the polypeptide are also provided in the present invention. Finally, the present invention provides kits for carrying out said methods wherein the kits comprise polynucleotides and/or antibodies capable of detecting the activity of alkylglycerol monooxygenase.

With the progress of the genome and cDNA sequencing projects, most if not all protein coding cDNAs in man and mouse have been characterised (1, 2). On the other hand, many enzyme activities have been described to a degree warranting assignment of an E.C. number, but still are orphans in the sense that they lack a sequence corresponding to the enzyme activity (3), and are expected to be promising therapeutic targets (4).

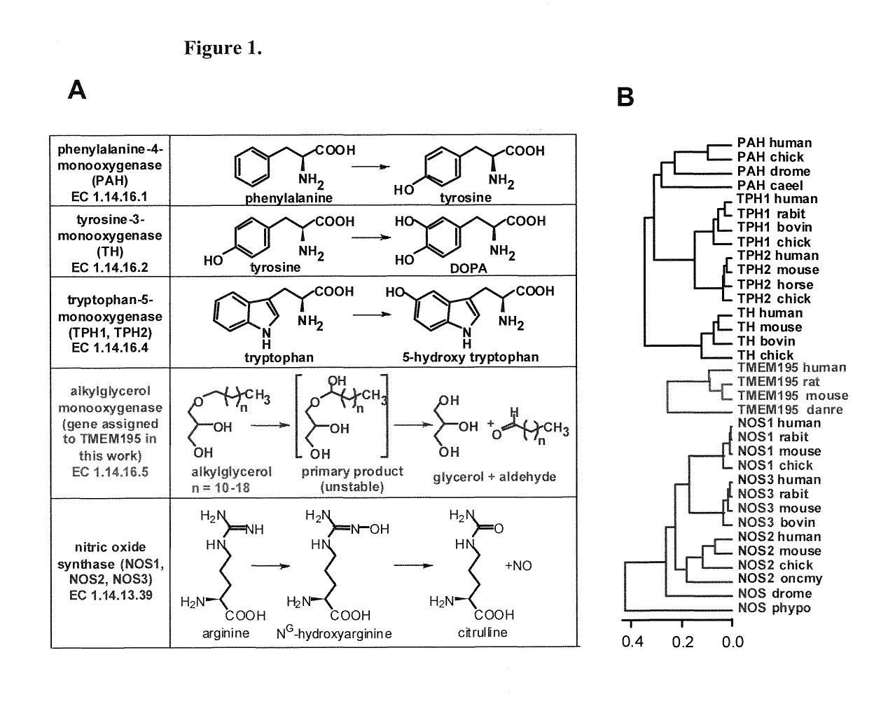

Tetrahydrobiopterin is a metabolite structurally related to the vitamins folic acid and riboflavin by sharing the common pterin (pyrimido[4,5-b]pyrazine) backbone. In contrast to the two vitamins which have to be taken up by the diet, however, tetrahydrobiopterin is synthesized in animals from guanosine triphosphate by the consecutive action of three enzymes (5). Five enzymatic reactions are known to depend essentially on the tetrahydrobiopterin cofactor (6) (FIG. 1A). In three of these reactions, a hydroxy function is introduced into the aromatic ring of phenylalanine, tyrosine and tryptophan by aromatic amino acid hydroxylases, which are required for the degradation of phenylalanine and for the biosynthesis of catecholamines and serotonin, important neurotransmitters and metabolism regulators. The fourth enzymatic reaction requiring tetrahydrobiopterin is catalyzed by nitric oxide synthases, which occur in three isoforms (7). After hydroxylation of the guanidino nitrogen of L-arginine in a first step, this reaction yields the radical gas nitric oxide and citrulline (8, 9). Nitric oxide synthases are required for a number of physiological processes such as blood pressure regulation, neurotransmission and host defense against pathogens (10-12). The fifth tetrahydrobiopterin-dependent enzymatic reaction catalyzed by alkylglycerol monooxygenase (glyceryl ether monooxygenase, EC 1.14.16.5) has been first described already in 1964 (13). Despite several attempts to purify and characterize this membrane bound protein (14), it still belonged to the currently 1187 enzymes lacking sequence assignment which are called orphan enzymes (15). Alkylglycerol monooxygenase is the only enzyme known to cleave the O-alkyl ether bond in alkylglycerols, yielding an aldehyde and a glycerol derivative. The aldehyde is detoxified by conversion to the corresponding acid by fatty aldehyde dehydrogenase (EC 1.2.1.48, gene symbol ALDH3A2). Tetrahydrobiopterin leaves the reaction as “quinoid” 6,7[8H]-dihydrobiopterin (14) and is recycled to tetrahydrobiopterin by quinoid dihydropteridine reductase (FIG. 3, EC 1.5.1.34, gene symbol QDPR). The formation of 6,7[8H]-dihydrobiopterin from the initial enzymatic product formed from tetrahydrobiopterin may be facilitated by 4a-carbinolamine dehydratase (EC 4.2.1.96, PCBD1) like for aromatic amino acid hydroxylases (16), but this has not yet been demonstrated for alkylglycerol monooxygenase.

One of these currently 280 human orphan enzymes is alkylglycerol monooxygenase (glyceryl ether monooxygenase, E.C. 1.14.16.5). It is one of only five enzyme reactions which are known to require tetrahydrobiopterin as a cofactor.

While the other four reactions (phenylalanine hydroxylase, tyrosine hydroxylase, tryptophan hydroxylases and nitric oxide synthases) are well characterized, and sequences and genes are known, little is known about alkylglycerol monooxygenase although its activity had been first described as early as 1964 and several attempts had been made to purify and study this enzyme (13; 17-21). FIG. 1A shows formulae and gene symbols of the currently known five tetrahydrobiopterin dependent reactions.

Alkylglycerols are abundant throughout the body, as is alkylglycerol monooxygenase (22). An alkylglycerol derivative constitutes the terminal lipid in the glycosylphosphatidyl (GPI) anchor used to attach many proteins to membranes. Alkylglycerol derivatives occur in a variety of lipid and phospholipid species, which have been characterized only to some but not all details. From the study of mice deficient in alkylglycerol biosynthesis, several important physiological functions have been deduced (23-25). These include spermatogenesis, protection of eyes from cataract formation, and central nervous system myelination.

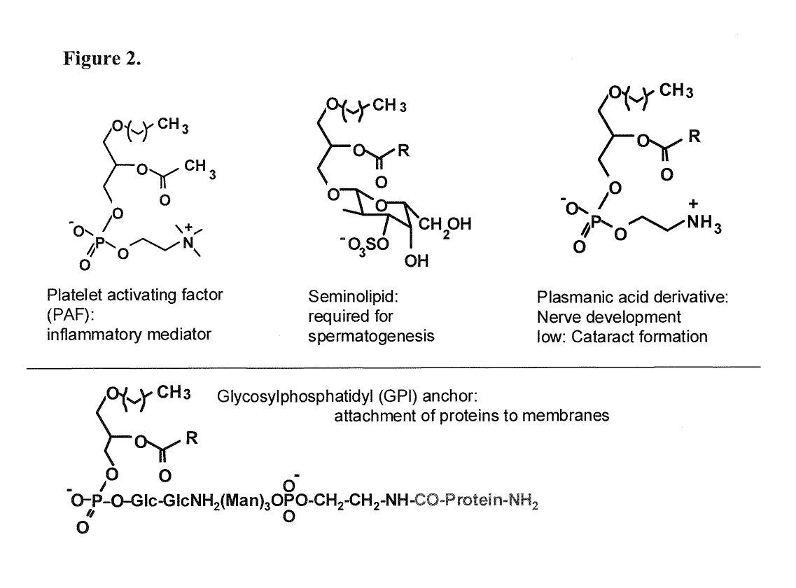

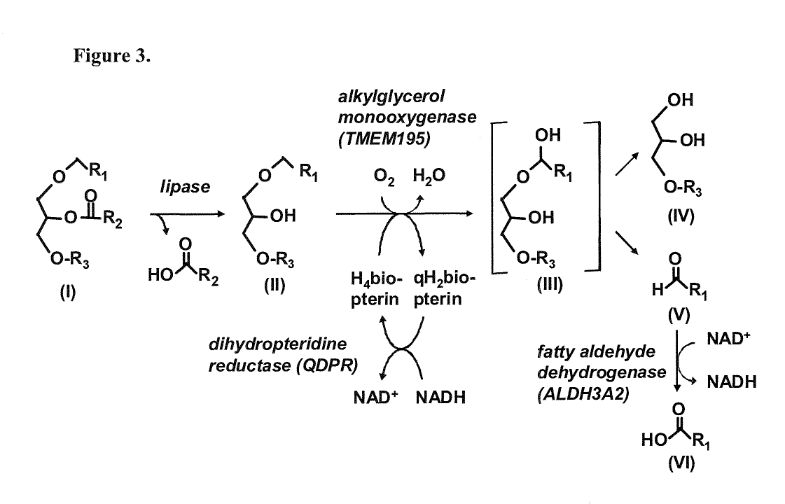

Another interesting aspect of alkylglycerols is their impact on signal transduction. In rat renal mesangial cells, alkylacylglycerols inhibit the diacylglycerol-mediated activation of protein kinase C isoforms. The differential effect of interleukin-1 and endothelin on the cells can be explained by the formation of alkylacylglycerols after treatment of cells by interleukin 1 (26). The antiproliferative action of etherlipid antitumor agents such as edelfosine may be caused by a similar mechanism, in addition to the suggested inhibition of phospholipid biosynthesis and apoptosis induction (27). FIGS. 2 and 3 show some examples of biologically active etherlipids, and the biochemistry of their degradation.

Investigation of the substrate specificity of alkylglycerol monooxygenase showed that the optimal alkyl length at R1 (FIG. 3) is 12 to 20 carbon atoms in total, that no double bond is accepted adjacent to the ether bond, that a free hydroxyl is required at C2 and that no restriction at the substitution of C3 is observed (14). C3 may even be missing (20). After the review on the biochemistry and substrate specificity of alkylglycerol monooxygenase by Armarego's group in 1998 (10), no further scientific paper appeared in the literature until a novel, robust and highly sensitive assay of the enzyme has been developed (22). With this, it has been able to observe widespread distribution of alkylglycerol monooxygenase activity in rat tissues. The biochemistry of the enzyme with respect to the effect of iron complexation by 1,10-phenanthroline could be characterized and the handling of tetrahydrobiopterin and two analogues of the cofactor. This led to the conclusion that alkylglycerol monooxygenase biochemically resembles aromatic amino acid hydroxylases which use non-heme iron for catalysis, rather than nitric oxide synthases which use heme iron for catalysis (28).

Despite the long standing practice with the purification of difficult enzymes like nitric oxide synthase from Physarum polycephalum (29, 30), the inventors of the present invention were thus far not able to purify the alkylglycerol monooxygenase protein from rat liver, the source with by far the highest specific activity. Despite detailed testing of more than a dozen detergents, the protein could not have even been solubilized to apparent homogeneity in gel filtration, but also at the best conditions it has been found still aggregated to a large extent. Although an in-gel assay for testing in gel slices of another, related membrane bound enzyme, fatty aldehyde dehydrogenase, could be developed (31), a similar attempt for alkylglycerol monooxygenase proved unsuccessful. The membrane enzyme fatty aldehyde dehydrogenase could immediately be purified from rat liver microsomes using published protocols, but alkylglycerol monooxygenase could not be enriched, although detergent combinations and stabilizing additives that let the activity survive three consecutive columns have been found. Therefore, after several years of intense trials, the inventors of the present invention had to admit that one may not succeed in purifying the native alkylglycerol monooxygenase protein in its active form.

Consequently, it is recognized in the art that there is a need to identify orphan molecules (i.e. molecules that lack a sequence corresponding to the enzyme activity) since they are expected to be promising therapeutic targets. Accordingly, the problem underlying the present invention is the provision of means and methods to treat diseases with an aberrant expression of alkylglycerol monooxygenase.

This technical problem is solved by the embodiments provided herein and as characterized in the claims. Specifically and in accordance with the present invention, a solution to this technical problem is achieved by providing a pharmaceutical composition comprising a nucleic acid molecule encoding a alkylglycerol monooxygenase (TMEM 195; glyceryl ether monooxygenase; EC 1.14.16.5) comprising a polynucleotide selected from the group consisting of: (a) a polynucleotide sequence as shown in SEQ ID NO:1 or a fragment thereof; (b) a polynucleotide sequence encoding a polypeptide as shown in SEQ ID NO:2 or a fragment thereof; (c) a polynucleotide sequence which has at least 80% identity to the polynucleotides as defined in (a) or (b) encoding a functional alkylglycerol monooxygenase or a fragment thereof; (d) a polynucleotide sequence which hybridizes to the polynucleotide sequence of any one of (a) to (c) and whereby the coding strand encodes a functional alkylglycerol monooxygenase or a fragment thereof; (e) a polynucleotide sequence encoding a polypeptide as encoded by the nucleotide sequence of any one of (a) to (d) wherein at least one amino acid is deleted, substituted, inserted or added and whereby said polynucleotide encodes a alkylglycerol monooxygenase or a fragment thereof; (f) a polynucleotide sequence being degenerate as a result of the genetic code to the nucleotide sequence as defined in any one of (a) to (e); and (g) the complementary strand of the polynucleotide of any one of (a) to (f).

In the context of the present invention, the term “alkylglycerol monooxygenase” relates to a polypeptide with tetrahydrobiopterin-dependent alkylglycerol monooxygenase activity and relates to a polypeptide that catalyzes the fifth tetrahydrobiopterin-dependent enzymatic reaction as already described above, i.e. the alkylglycerol monooxygenase (glycerylether monooxygenase, EC 1.14.16.5). In the context of the present invention, this polypeptide is also referred to as TMEM195 (transmembrane protein 195), glyceryl ether monooxygenase and EC 1.14.16.5 and relates to sequences disclosed herein below. As exemplified in the appended examples, the alkylglycerol monooxygenase TMEM195 provided herein include inter alia the following functions and activities, i.e. the capability of having alkylglycerol monooxygenase enzyme activity as described herein below. For testing the functional alkylglycerol monooxygenase activity, assays provided herein below and as exemplified in the appended examples may be used. Structural features of the alkylglycerol monooxygenase TMEM195 of the present invention are exemplified in the appended examples.

The coding regions of the alkylglycerol monooxygenase TMEM195 of the present invention (glycerylether monooxygenase, EC 1.14.16.5) or functional fragments thereof are known in the art and comprise, inter alia, the GenBank entries for Pan troglodytes, XP_{—}518978.2, XM_{—}518978.2; Canis lupus familiaris, XP_{—}532481.2, XM_{—}518978.2; Bos taurus, XP_{—}869752.2, XM_{—}864659.3; Rattus norvegicus, NP_{—}001129371.1, NM_{—}001135899.1; Gallus gallus, XP_{—}001235521.1, XM_{—}001235520.1; Danio rerio, NP_{—}998048.1, NM 212883.1; Caenorhabditis elegans, NP_{—}499664.2, NM_{—}067263.4; Macaca mulatta, XP_{—}001105641.1, XM_{—}001105641.1; Equus caballus, XP_{—}001495726.1, XM_{—}001495676.1; Oryctolagus cuniculus, XP_{—}002720904.1, XM_{—}002720858.1; Mus musculus, NP_{—}848882.2, NM_{—}178767.4; Monodelphis domestica, XP_{—}001374227.1, XM_{—}001374190.1; Xenopus (Silurana) tropicalis, NP_{—}001011313.1, NM_{—}001011313.1; Branchiostoma floridae, XP_{—}002593852.1, XM_{—}002593806.1; Strongylocentrotus purpuratus, XP_{—}784522.1, XM_{—}779429.2; Ciona intestinalis, XP_{—}002128466.1, XM_{—}002128430.1; Tribolium castaneum, XP_{—}969001.1, XM_{—}963908.2; Nasonia vitripennis, XP_{—}001603646.1, XM_{—}001603596.1; Apis mellifera, XP_{—}397032.3, XM_{—}397032.3. The person skilled in the art may easily deduce the relevant coding region of the alkylglycerol monooxygenase TMEM195 of the present invention in these GenBank entries, which may also comprise the entry of genomic DNA as well as mRNA/cDNA.

The human alkylglycerol monooxygenase TMEM195 cDNA is given in SEQ ID NO:3. Thus, in particular, wild type human alkylglycerol monooxygenase TMEM195 may be encoded by the following nucleic acid sequence:

(SEQ ID NO: 1)
ATGAAGAACCCAGAAGCCCAGCAGGATGTTTCAGTTTCCCAGGGATTTCGCATGTTGTTTTACACGATGA
AACCCAGTGAAACTTCATTCCAAACATTAGAAGAGGTGCCTGATTATGTAAAAAAGGCAACTCCATTTTT
CATTTCTTTGATGCTGCTTGAACTTGTTGTCAGCTGGATTCTCAAAGGAAAGCCACCAGGTCGCCTGGAT
GATGCTTTAACGTCAATCTCAGCTGGTGTTCTGTCTCGACTTCCAAGTCTATTTTTCAGGAGCATTGAAC
TGACCAGTTATATTTATATCTGGGAGAACTACAGGCTGTTCAATTTGCCTTGGGATTCTCCATGGACTTG
GTATTCAGCCTTCTTAGGAGTTGACTTTGGCTACTACTGGTTCCATCGTATGGCTCATGAAGTTAATATT
ATGTGGGCCGGACACCAAACACATCATAGTTCTGAAGACTATAACTTATCCACAGCACTGAGACAGTCTG
TCCTCCAGATATATACTTCCTGGATTTTCTACTCTCCCCTGGCCCTCTTCATACCCCCTTCAGTATATGC
TGTTCATCTTCAATTCAATCTTCTTTACCAATTTTGGATCCATACAGAGGTCATCAATAACCTTGGTCCT
TTGGAACTGATTCTTAATACTCCTAGCCATCATAGGGTTCATCATGGCAGAAATCGTTATTGCATAGACA
AAAATTATGCTGGTGTTCTTATTATTTGGGATAAAATTTTTGGGACATTTGAAGCAGAAAATGAAAAAGT
TGTATATGGCTTAACACATCCCATTAATACATTTGAACCAATCAAAGTGCAGTTCCATCACTTATTTTCC
ATATGGACTACATTCTGGGCCACACCTGGATTCTTCAATAAGTTTTCTGTCATATTTAAGGGACCGGGAT
GGGGTCCAGGTAAACCAAGACTTGGTCTCAGTGAAGAAATTCCAGAGGTCACCGGCAAAGAAGTTCCCTT
CTCATCATCTTCATCTCAGCTATTAAAGATATATACAGTTGTACAGTTTGCTCTGATGTTGGCATTTTAT
GAAGAGACCTTTGCAGATACAGCTGCACTGTCGCAAGTTACTCTCCTTCTGAGGGTTTGCTTCATTATCC
TGACCTTGACTTCCATTGGATTTCTTCTGGATCAAAGACCCAAGGCAGCTATTATGGAAACTCTCCGTTG
CTTGATGTTCTTAATGCTGTACCGATTTGGTCACCTGAAGCCTCTTGTCCCTTCATTGTCATCTGCTTTT
GAGATTGTTTTTTCCATTTGCATTGCTTTCTGGGGAGTTAGAAGCATGAAACAACTCACCTCTCACCCTT
GGAAATAA,

which corresponds to the following amino acid sequence:

(SEQ ID NO: 2)
MKNPEAQQDVSVSQGFRMLFYTMKPSETSFQTLEEVPDYVKKATPFFISLMLLELVVSWILKGKPPGRLD
DALTSISAGVLSRLPSLFFRSIELTSYIYIWENYRLFNLPWDSPWTWYSAFLGVDFGYYWFHRMAHEVNI
MWAGHQTHHSSEDYNLSTALRQSVLQIYTSWIFYSPLALFIPPSVYAVHLQFNLLYQFWIHTEVINNLGP
LELILNTPSHHRVHHGRNRYCIDKNYAGVLIIWDKIFGTFEAENEKVVYGLTHPINTFEPIKVQFHHLFS
IWTTFWATPGFFNKFSVIFKGPGWGPGKPRLGLSEEIPEVTGKEVPFSSSSSQLLKIYTVVQFALMLAFY
EETFADTAALSQVTLLLRVCFIILTLTSIGFLLDQRPKAAIMETLRCLMFLMLYRFGHLKPLVPSLSSAF
EIVFSICIAFWGVRSMKQLTSHPWK.

The cDNA of the human alkylglycerol monooxygenase TMEM195 corresponds to the following sequence:

(SEQ ID NO: 3)
CTCTCTACACAGAATCGGCTGTTGAGTGTGCTCTCAGTGGAGCTTTGGTTTTAGCTGTTCTCTGACAAAG
AGCTTGTTCTGAGCTGCACATCTCGTCCTCTTTGTTCAGCCTCAGGCTTCAAGCATTGAATCCTAAATAT
TCTCCAGCTGGGAATCAGACAAGGGCAGAAATGAAGAACCCAGAAGCCCAGCAGGATGTTTCAGTTTCCC
AGGGATTTCGCATGTTGTTTTACACGATGAAACCCAGTGAAACTTCATTCCAAACATTAGAAGAGGTGCC
TGATTATGTAAAAAAGGCAACTCCATTTTTCATTTCTTTGATGCTGCTTGAACTTGTTGTCAGCTGGATT
CTCAAAGGAAAGCCACCAGGTCGCCTGGATGATGCTTTAACGTCAATCTCAGCTGGTGTTCTGTCTCGAC
TTCCAAGTCTATTTTTCAGGAGCATTGAACTGACCAGTTATATTTATATCTGGGAGAACTACAGGCTGTT
CAATTTGCCTTGGGATTCTCCATGGACTTGGTATTCAGCCTTCTTAGGAGTTGACTTTGGCTACTACTGG
TTCCATCGTATGGCTCATGAAGTTAATATTATGTGGGCCGGACACCAAACACATCATAGTTCTGAAGACT
ATAACTTATCCACAGCACTGAGACAGTCTGTCCTCCAGATATATACTTCCTGGATTTTCTACTCTCCCCT
GGCCCTCTTCATACCCCCTTCAGTATATGCTGTTCATCTTCAATTCAATCTTCTTTACCAATTTTGGATC
CATACAGAGGTCATCAATAACCTTGGTCCTTTGGAACTGATTCTTAATACTCCTAGCCATCATAGGGTTC
ATCATGGCAGAAATCGTTATTGCATAGACAAAAATTATGCTGGTGTTCTTATTATTTGGGATAAAATTTT
TGGGACATTTGAAGCAGAAAATGAAAAAGTTGTATATGGCTTAACACATCCCATTAATACATTTGAACCA
ATCAAAGTGCAGTTCCATCACTTATTTTCCATATGGACTACATTCTGGGCCACACCTGGATTCTTCAATA
AGTTTTCTGTCATATTTAAGGGACCGGGATGGGGTCCAGGTAAACCAAGACTTGGTCTCAGTGAAGAAAT
TCCAGAGGTCACCGGCAAAGAAGTTCCCTTCTCATCATCTTCATCTCAGCTATTAAAGATATATACAGTT
GTACAGTTTGCTCTGATGTTGGCATTTTATGAAGAGACCTTTGCAGATACAGCTGCACTGTCGCAAGTTA
CTCTCCTTCTGAGGGTTTGCTTCATTATCCTGACCTTGACTTCCATTGGATTTCTTCTGGATCAAAGACC
CAAGGCAGCTATTATGGAAACTCTCCGTTGCTTGATGTTCTTAATGCTGTACCGATTTGGTCACCTGAAG
CCTCTTGTCCCTTCATTGTCATCTGCTTTTGAGATTGTTTTTTCCATTTGCATTGCTTTCTGGGGAGTTA
GAAGCATGAAACAACTCACCTCTCACCCTTGGAAATAACCTGAATTTGTACATAATTCTCTTCTTTTAAT
GAGTTGTCCACACGCATATTATGACTGCATATTAAAATGTAATTATTTTATGTAATGCTTATATGAACTA
TTTCTTCAATGAAAAGTAAAATTACTTATTTACTATTGTTTGCCTTTCACATTTGTTATTTTCTATTAAA
AATTAAAGTCAGTTTTGGTTACTTCCCCCCTTTACTACAATTAAAAAAAGATTTCAAATATAATGATGTT
ATATTAACTGATAGCCTTATATGACAAGTATAAAAAGAAGGGATGAAACTTAAAAACAGTAAAAACAAGA
AGGAATATTGCCTTTACATCAATTTGAAAACAATGTTTCCTTTGATGTTTGCTAAAATTATGCATAGATA
CATGTTTGTAGTCATAAAAATGTATTACATTGGTTGTCTTCCTAAGGCCACAGTTACCTTTGCAATCCAT
ATAACCTAAGAAGCTGCATTCCAGAAAAAGACATCACTGAGGCCAGGCGCGGTGGCTCACCCCTGTAATC
CCAGCACTTTGTGGGGCTGAGGTGGGCGGATCATGAGGTCCAGAGATAGAGACCATCCTGGCCAACATGG
TGAAGTCCTGTCTCTACTAAAAATATAAAAATTTAGCTGGACATGGTGGTGTGCGCCTGTAGTCCCAGCT
ACTCTGGAGGCTGAGGCAGGAGAATCGCTTGAACCTGGGAGGCAGAAGTTGGAGTGAGTGGAGATTGCAC
CACTGCACTCCAGCCTGGGCGACAGAGCGAGACTCCGTCTCAAAGAAAAAAGACATCACTGAAAGAAAAA
TGAACAGAATTTGTCAGAATTAGTTTTTTCAACAGGTTACTTTGTCATACATTTCTCTAATATGCTTGGT
CAATTTGTTTTGGCAGACTGGGCAGCATGCAGCAATTCTGCATTATTTAAAGTTATCAGAACAATGTTAA
TTCTCTAAATAAAATTACCCAAGGT.

Accordingly, the alkylglycerol monooxygenase TMEM195 molecules to be employed in the context of the present invention comprise, but are not limited to the molecules encoded by the nucleic acid molecules as described herein. Also envisaged are alkylglycerol monooxygenase TMEM195 orthologs which are at least 60%, 70%, 80%, 90%, 95%, 96%, 97%, 98% or 99% identical to nucleic acid sequence as shown in SEQ ID NO: 1. These alkylglycerol monooxygenase TMEM195 molecules as referred here are defined as molecules that are capable of acting as a functional alkylglycerol monooxygenase as described herein above and below. These functions and activities include, inter alia, the capability of having alkylglycerol monooxygenase enzyme activity as described herein above and below. For testing the functional alkylglycerol monooxygenase activity, assays provided herein below and as exemplified in the appended examples may be used. Furthermore envisaged are alkylglycerol monooxygenase TMEM195 orthologs which are at least 60%, 70%, 80%, 90%, 95%, 96%, 97%, 98% or 99% identical to the amino acid sequence as shown in SEQ ID NO: 2 and being capable of acting as a functional alkylglycerol monooxygenase as described herein above and below. In addition, the term “alkylglycerol monooxygenase TMEM195 ortholog” comprises molecules which are at least 60%, more preferably at least 80% and most preferably at least 90% homologous to the polypeptide as shown in SEQ ID NO: 2 and being capable of acting as a functional alkylglycerol monooxygenase as described herein above and below.

In order to determine whether a nucleic acid sequence has a certain degree of identity to a nucleic acid encoding alkylglycerol monooxygenase TMEM195 orthologs, the skilled person can use means and methods well known in the art, e.g. alignments, either manually or by using computer programs such as those mentioned herein below in connection with the definition of the term “hybridization” and degrees of homology.

The term “hybridization” or “hybridizes” as used herein may relate to hybridizations under stringent or non-stringent conditions. If not further specified, the conditions are preferably non-stringent. Said hybridization conditions may be established according to conventional protocols described, e.g., in Sambrook, Russell “Molecular Cloning, A Laboratory Manual”, Cold Spring Harbor Laboratory, N.Y. (2001); Ausubel, “Current Protocols in Molecular Biology”, Green Publishing Associates and Wiley Interscience, N.Y. (1989), or Higgins and Hames (Eds.) “Nucleic acid hybridization, a practical approach” IRL Press Oxford, Washington D.C., (1985). The setting of conditions is well within the skill of the artisan and can be determined according to protocols described in the art. Thus, the detection of only specifically hybridizing sequences will usually require stringent hybridization and washing conditions such as 0.1×SSC, 0.1% SDS at 65° C. Non-stringent hybridization conditions for the detection of homologous or not exactly complementary sequences may be set at 6×SSC, 1% SDS at 65° C. As is well known, the length of the probe and the composition of the nucleic acid to be determined constitute further parameters of the hybridization conditions. Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility. Hybridizing nucleic acid molecules also comprise fragments of the above described molecules. Such fragments may represent nucleic acid sequences which code for alkylglycerol monooxygenase TMEM195 or a functional fragment thereof which have a length of at least 12 nucleotides, preferably at least 15, more preferably at least 18, more preferably of at least 21 nucleotides, more preferably at least 30 nucleotides, even more preferably at least 40 nucleotides and most preferably at least 60 nucleotides. Furthermore, nucleic acid molecules which hybridize with any of the aforementioned nucleic acid molecules also include complementary fragments, derivatives and allelic variants of these molecules. Additionally, a hybridization complex refers to a complex between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an anti-parallel configuration. A hybridization complex may be formed in solution (e.g., Cot or Rot analysis) or between one nucleic acid sequence present in solution and another nucleic acid sequence immobilized on a solid support (e.g., membranes, filters, chips, pins or glass slides to which, e.g., cells have been fixed). The terms “complementary” or “complementarity” refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between single-stranded molecules. The degree of complementarity between nucleic acid strands has significant effects on the efficiency and strength of hybridization between nucleic acid strands. This is of particular importance in amplification reactions, which depend upon binding between nucleic acids strands.

The term “hybridizing sequences” preferably refers to sequences which display a sequence identity of at least 40%, preferably at least 50%, more preferably at least 60%, even more preferably at least 70%, particularly preferred at least 80%, more particularly preferred at least 90%, even more particularly preferred at least 95% and most preferably at least 97% identity with a nucleic acid sequence as described above (i.e. SEQ ID NO: 1) encoding alkylglycerol monooxygenase TMEM 195 or a functional fragment thereof and being capable of acting as a functional alkylglycerol monooxygenase as described herein above and below and tests are described for assaying the alkylglycerol monooxygenase activity as described in detail below and as exemplified in the appended examples. Moreover, the term “hybridizing sequences” preferably refers to sequences encoding alkylglycerol monooxygenase TMEM195 or a functional fragment thereof having a sequence identity of at least 40%, preferably at least 50%, more preferably at least 60%, even more preferably at least 70%, particularly preferred at least 80%, more particularly preferred at least 90%, even more particularly preferred at least 95% and most preferably at least 97% identity with an amino acid sequence of the alkylglycerol monooxygenase TMEM195 sequences as described herein (i.e. SEQ ID NO: 2) and being as an capable of acting as a functional alkylglycerol monooxygenase as described herein above and below.

In accordance with the present invention, the term “identical” or “percent identity” in the context of two or more nucleic acid or amino acid sequences, refers to two or more sequences or subsequences that are the same, or that have a specified percentage of amino acid residues or nucleotides that are the same (e.g., 60% or 65% identity, preferably, 70-95% identity, more preferably at least 95% identity with the nucleic acid sequences of, e.g., SEQ ID NO: 1 or with the amino acid sequence of, e.g., SEQ ID NO: 2 and being capable of acting as a functional alkylglycerol monooxygenase as described herein above and below), when compared and aligned for maximum correspondence over a window of comparison, or over a designated region as measured using a sequence comparison algorithm as known in the art, or by manual alignment and visual inspection. Sequences having, for example, 60% to 95% or greater sequence identity are considered to be substantially identical. Such a definition also applies to the complement of a test sequence. Preferably, the described identity exists over a region that is at least about 15 to 25 amino acids or nucleotides in length, more preferably, over a region that is about 50 to 100 amino acids or nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art.

Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul, (1997) Nucl. Acids Res. 25:3389-3402; Altschul (1993) J. Mol. Evol. 36:290-300; Altschul (1990) J. Mol. Biol. 215:403-410). The BLASTN program for nucleic acid sequences uses as defaults a word length (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff (1989) PNAS 89:10915) uses alignments (B) of 50, expectation (E) of 10, M=5, N=4, and a comparison of both strands.

Moreover, the present invention also relates to nucleic acid molecules whose sequence is being degenerate in comparison with the sequence of an above-described hybridizing molecule. When used in accordance with the present invention the term “being degenerate as a result of the genetic code” means that due to the redundancy of the genetic code different nucleotide sequences code for the same amino acid.

In order to determine whether an amino acid residue or nucleotide residue in a nucleic acid sequence corresponds to a certain position in the amino acid sequence of, e.g., SEQ ID NO: 2 or nucleotide sequence of e.g. SEQ ID NO: 1, the skilled person can use means and methods well-known in the art, e.g., alignments, either manually or by using computer programs such as those mentioned further down below in connection with the definition of the term “hybridization” and degrees of homology.

For example, BLAST 2.0, which stands for Basic Local Alignment Search Tool BLAST (Altschul (1997), loc. cit.; Altschul (1993), loc. cit.; Altschul (1990), loc. cit.), can be used to search for local sequence alignments. BLAST, as discussed above, produces alignments of both nucleotide and amino acid sequences to determine sequence similarity. Because of the local nature of the alignments, BLAST is especially useful in determining exact matches or in identifying similar sequences. The fundamental unit of BLAST algorithm output is the High-scoring Segment Pair (HSP). An HSP consists of two sequence fragments of arbitrary but equal lengths whose alignment is locally maximal and for which the alignment score meets or exceeds a threshold or cut-off score set by the user. The BLAST approach is to look for HSPs between a query sequence and a database sequence, to evaluate the statistical significance of any matches found, and to report only those matches which satisfy the user-selected threshold of significance. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the program output.

Analogous computer techniques using BLAST (Altschul (1997), loc. cit.; Altschul (1993), loc. cit.; Altschul (1990), loc. cit.) are used to search for identical or related molecules in nucleotide databases such as GenBank or EMBL. This analysis is much faster than multiple membrane-based hybridizations. In addition, the sensitivity of the computer search can be modified to determine whether any particular match is categorized as exact or similar. The basis of the search is the product score which is defined as:

$$\frac{\% sequence identity \times \% maximum BLAST score}{100}$$

and it takes into account both the degree of similarity between two sequences and the length of the sequence match. For example, with a product score of 40, the match will be exact within a 1-2% error; and at 70, the match will be exact. Similar molecules are usually identified by selecting those which show product scores between 15 and 40, although lower scores may identify related molecules. Another example for a program capable of generating sequence alignments is the CLUSTALW computer program (Thompson (1994) Nucl. Acids Res. 2:4673-4680) or FASTDB (Brutlag (1990) Comp. App. Biosci. 6:237-245), as known in the art.

Accordingly, and in a further aspect, the present invention relates to a vector comprising the nucleic acid molecules described herein and a recombinant host cell comprising the nucleic acid molecules and/or the vector.

The term “vector” as used herein particularly refers to plasmids, cosmids, viruses, bacteriophages and other vectors commonly used in genetic engineering. In a preferred embodiment, the vectors of the invention are suitable for the transformation of cells, like fungal cells, cells of microorganisms such as yeast or bacterial cells or animal cells. The vectors as well as the host cells of the present invention are particularly useful in the recombinant expression of the polypeptides (alkylglycerol monooxygenase TMEM195 and fragments thereof) of the present invention.

In a further aspect, the recombinant host cell of the present invention is capable of expressing or expresses the polypeptide encoded by the polynucleotide sequence of this invention. In a specific embodiment, the “polypeptide” comprised in the host cell may be a heterologous with respect to the origin of the host cell. An overview of examples of different expression systems to be used for generating the host cell of the present invention, for example the above-described particular one, is for instance contained in Glorioso et al. (1999), Expression of Recombinant Genes in Eukaryotic Systems, Academic Press Inc., Burlington, USA, Paulina Balbas and Argelia Lorence (2004), Recombinant Gene Expression: Reviews and Protocols, Second Edition Reviews and Protocols (Methods in Molecular Biology), Humana Press, USA.

The transformation or genetically engineering of the host cell with a nucleotide sequence or the vector according to the invention can be carried out by standard methods, as for instance described in Sambrook and Russell (2001), Molecular Cloning: A Laboratory Manual, CSH Press; Cold Spring Harbor, N.Y., USA. Moreover, the host cell of the present invention is cultured in nutrient media meeting the requirements of the particular host cell used, in particular in respect of the pH value, temperature, salt concentration, aeration, antibiotics, vitamins, trace elements etc.

The term “recombinant host cell”, as used herein, relates to a host cell, genetically engineered with the nucleotide sequence of the present invention or comprising the vector or the polypeptide or a fragment thereof of the present invention.

Generally, the host cell of the present invention may be a prokaryotic or eukaryotic cell comprising the nucleotide sequence, the vector and/or the polypeptide of the invention or a cell derived from such a cell and containing the nucleotide sequence, the vector and/or the polypeptide of the invention. In a preferred embodiment, the host cell comprises, for example due to genetic engineering, the nucleotide sequence or the vector of the invention in such a way that it contains the nucleotide sequences of the present invention integrated into the genome. Non-limiting examples of such a host cell of the invention (but also the host cell of the invention in general) may be a bacterial, yeast, fungus, plant, animal or human cell.

In accordance with the above, the invention relates in a further embodiment to a method for producing a polypeptide provided herein, comprising culturing the recombinant host cell under such conditions that the polypeptide is expressed, and recovering the polypeptide.

The term “such conditions”, as used herein, refers to culture conditions of recombinant host cells in order to express and recover polypeptides, preferably heterologous polypeptides. These conditions are well known to a person skilled in the art, as for instance described in Sambrook and Russell (2001), Molecular Cloning: A Laboratory Manual, CSH Press, Cold Spring Harbor, N.Y., USA.

In general, the polypeptide of this invention may, in addition to the alkylglycerol monooxygenase TMEM195 of the invention and as defined herein, comprise (a) further polypeptide(s), i.e. (a) polypeptide(s) being heterologous with respect to the polypeptides of the present invention. One skilled in the art will be aware that the heterologous polypeptide can be expressed by prokaryotic (e.g. bacteria) or eukaryotic cell (e.g. 293 cells or CHO cells). The present invention also relates to a fusion protein (and a nucleic acid molecule encoding the fusion protein). For example, the further/heterologous polypeptide(s) may particularly be suitable for a potentiated or increased production for the polypeptide of the present invention (for example, in a cell). The further/heterologous polypeptide(s) may, for example, comprise a protein fragment or peptide, an entire functional moiety, or an entire protein sequence which can be designed to be used in purifying the fusion protein, for example either with antibodies or with affinity purification specific for the further/heterologous polypeptide. Likewise, physical properties of the additional polypeptide, protein fragment, peptide (and the like) can be exploited to allow selective purification of the heterologous polypeptide and, hence, also the polypeptide of the present invention.

As a fusion protein, the polypeptide of the present invention, i.e., e.g., as shown in SEQ ID NO: 2, may also include a reporter or reporter construct being expressible in a cell, a tissue, a cell culture, tissue culture, animals or plants. Thus, the polypetide of the present invention can be easily identified and measured by the skilled person, in order to determine whether the polypeptide has been expressed in the cell, animals or plants.

Commonly used reporter genes that induce visually identifiable characteristics usually involve fluorescent and luminescent proteins; examples include the gene that encodes jellyfish green fluorescent protein (GFP), which causes cells that express it to glow green under blue light, the enzyme luciferase, which catalyzes a reaction with luciferin to produce light, and the red fluorescent protein from the gene dsRed. Another common reporter in bacteria is the lacZ gene, which encodes the protein β-galactosidase. This enzyme causes bacteria expressing the gene to appear blue when grown on a medium that contains the substrate analog X-gal (an inducer molecule such as Isopropyl β-D-1-thiogalactopyranoside (IPTG) is also needed under the native promoter).

One of skill in the art will recognize that the particular peptide/protein fragment etc. is chosen with the purification scheme in mind. As a non-limiting example, His tags, GST, and maltose-binding protein represent peptides that have readily available affinity columns to which they can be bound and eluted. Thus, where the peptide is an N-terminal His tag such as hexahistidine (His.sub.6 tag), the heterologous protein can be purified using a matrix comprising a metal-chelating resin, for example, nickel nitrilotriacetic acid (Ni-NTA), nickel iminodiacetic acid (Ni-IDA), and cobalt-containing resin (Co-resin). See, for example, Steinert et al. (1997) QIAGEN News 4:11-15. Where the peptide is GST, the heterologous protein can be purified using a matrix comprising glutathione-agarose beads (Sigma or Pharmacia Biotech); where the protein fragment is a maltose-binding protein (MBP), the heterologous protein can be purified using a matrix comprising an agarose resin derivatized with amylose.

In a further embodiment, the present invention refers to a pharmaceutical composition having the amino acid sequence encoded by the nucleic acid molecule of the present invention, or fragment thereof having the amino acid sequence encoded by the nucleic acid molecules which encodes the alkylglycerol monooxygenase TMEM195 wherein said fragment is a alkylglycerol monooxygenase TMEM195. Furthermore, the pharmaceutical composition comprises the polypeptide is obtainable by the above-mentioned method. Polypeptides of the present invention have been described in detail herein above.

In a particular embodiment, the present invention relates to an antibody and the use thereof that specifically binds to the polypeptide or fragments thereof as shown in SEQ ID NO: 2. Moreover, said antibody can be used for the purification and detection of said polypeptide. The term “antibody” is well known in the art.

In context of the present invention, the term “antibody” as used herein relates in particular to full immunoglobulin molecules as well as to parts of such immunoglobulin molecules substantially retaining binding specificity. Furthermore, the term relates to modified and/or altered antibody molecules, like chimeric and humanized antibodies, CDR-grafted antibodies, recombinantly or synthetically generated/synthesized antibodies and to intact antibodies as well as to antibody fragments thereof, like, separated light and heavy chains, Fab, Fab/c, Fv, Fab′, F(ab′)₂. The term “antibody” also comprises bifunctional antibodies, trifunctional antibodies and antibody constructs, like single chain Fvs (scFv) or antibody-fusion proteins. Further “antibody” constructs are known in the art and comprised in the present invention. Techniques for the production of antibodies are well known in the art and described, e.g. in Howard and Bethell (2000) Basic Methods in Antibody Production and Characterization, Crc. Pr. Inc. Antibodies directed against a polypeptide according to the present invention can be obtained, e.g., by direct injection of the polypeptide (or a fragment thereof) into an animal or by administering the polypeptide (or a fragment thereof) to an animal. The antibody so obtained will then bind polypeptide (or a fragment thereof) itself. In this manner, even a fragment of the polypeptide can be used to generate antibodies binding the whole polypeptide, as long as said binding is “specific” as defined above.

These polypeptides are particularly useful in the preparation of specific antibodies and are provided herein for illustrative purposes.

With the normal skill of the person skilled in the art and by routine methods, the person skilled in the art can easily deduce from the sequences provided herein relevant epitopes (also functional fragments) of the polypeptides of the present invention which are useful in the generation of antibodies like polyclonal and monoclonal antibodies. However, the person skilled in the art is readily in a position to also provide for engineered antibodies like CDR-grafted antibodies or also humanized and fully human antibodies and the like.

Particularly preferred in the context of the present invention are monoclonal antibodies. For the preparation of monoclonal antibodies, any technique which provides antibodies produced by continuous cell line cultures can be used. Examples for such techniques include the hybridoma technique, the trioma technique, the human B-cell hybridoma technique and the EBV-hybridoma technique to produce human monoclonal antibodies (Shepherd and Dean (2000), Monoclonal Antibodies: A Practical Approach, Oxford University Press, Goding and Goding (1996), Monoclonal Antibodies: Principles and Practice—Production and Application of Monoclonal Antibodies in Cell Biology, Biochemistry and Immunology, Academic Pr Inc, USA).

The antibody derivatives can also be produced by peptidomimetics. Further, techniques described for the production of single chain antibodies (see, inter alia, U.S. Pat. No. 4,946,778) can be adapted to produce single chain antibodies specifically recognizing the polypeptide of the invention. Also, transgenic animals may be used to express humanized antibodies to the polypeptide of the invention.

As shown in the appended examples, the present invention also envisages the production of specific antibody against native polypeptides and recombinant polypeptides according to the invention. This production is based, for example, on the immunization of animals, like mice. However, also other animals for the production of antibody/antisera are envisaged within the present invention. For example, monoclonal and polyclonal antibodies can be produced by rabbit, mice, goats, donkeys and the like. The polynucleotide according to the invention as shown in SEQ ID NO:1 can be subcloned into an appropriated vector, wherein the recombinant polypeptide is to be expressed in an organism being able for an expression, for example in bacteria. Thus, the expressed recombinant protein can be intra-peritoneally injected into a mice and the resulting specific antibody can be, for example, obtained from the mice serum being provided by intra-cardiac blood puncture. The amount of obtained specific antibody can be quantified using an ELISA, which is also described herein below. Further methods for the production of antibodies are well known in the art, see, e.g. Harlow and Lane, “Antibodies, A Laboratory Manual”, CSH Press, Cold Spring Harbor, 1988.

The term “specifically binds”, as used herein, refers to a binding reaction that is determinative of the presence of the alkylglycerol monooxygenase TMEM195 protein and antibody in the presence of a heterogeneous population of proteins and other biologics.

Thus, under designated assay conditions, the specified antibodies and alkylglycerol monooxygenase TMEM195 proteins bind to one another and do not bind in a significant amount to other components present in a sample. Specific binding to a target analyte under such conditions may require a binding moiety that is selected for its specificity for a particular target analyte. A variety of immunoassay formats may be used to select antibodies specifically reactive with a particular antigen. For example, solid-phase ELISA immunoassays are routinely used to select monoclonal antibodies specifically immunoreactive with an analyte. See Shepherd and Dean (2000), Monoclonal Antibodies: A Practical Approach, Oxford University Press and/or Howard and Bethell (2000) Basic Methods in Antibody Production and Characterization, Crc. Pr. Inc. for a description of immunoassay formats and conditions that can be used to determine specific immunoreactivity. Typically a specific or selective reaction will be at least twice background signal to noise and more typically more than 10 to 100 times greater than background. The person skilled in the art is in a position to provide for and generate specific binding molecules directed against the novel polypeptides. For specific binding-assays it can be readily employed to avoid undesired cross-reactivity, for example polyclonal antibodies can easily be purified and selected by known methods (see Shepherd and Dean, loc. cit.).

The term “purification or detection”, as used herein, refers to a series of processes intended to isolate or detect a single type of protein from a complex mixture employing the “specifical binding” as defined above, that refers to a binding reaction that is determinative of the presence of the alkylglycerol monooxygenase TMEM195 protein and antibody in the presence of a heterogeneous population of proteins and other biologics. Protein purification or detection is vital for the characterisation of the function, structure and interactions of the protein of interest. The starting material, as a non-limiting example, can be a biological tissue or a microbial culture. The various steps in the purification or detection process may free the protein from a matrix that confines it, separate the protein and non-protein parts of the mixture, and finally separate the desired protein from all other proteins. Separation steps exploit differences in protein size, physico-chemical properties and binding affinity. Exemplary purification methods are also shown in appended Examples.

In summary, in accordance with the above and in relation with the embodiments of this invention, the present invention relates also to a pharmaceutical composition comprising the polynucleotide as shown in SEQ ID NO:1, the vector, the polypeptide as shown in Seq ID No 2 or the antibody. The pharmaceutical composition of the present invention may also comprise (functional) fragments of the polypeptides provided herein. Such pharmaceutical composition may, inter alia, be used in elicitating male infertility and to ameliorate both the neurodegeneration and the recent memory loss associated with Alzheimer's disease.

### Testis/Male Infertility:

Testes contain a complex ether lipid, the called seminolipid (23). Male mice deficient in the biosynthesis of ether lipids are infertile. Infertility of men is only understood in a minor part of the cases (O'Flynn O'Brien, K. L. et al., Fertil Steril 93(1):1-12 (2010)). Testis is an organ with high alkylglycerol monooxygenase activity. In cases where a partial inhibition of alkylglycerol biosynthesis may cause male infertility due to defective biosynthesis of seminolipid or its precursors, dimishing the activity of alkylglycerol has the potential to restore fertility. On the contrary, increasing the activity of alkylglycerolmonoxygenase in testis is expected to degrade seminolipid, and thereby elicit male infertility comparable to the situation found in mice lacking etherlipid biosynthesis (23).

### Overexpression/Upregulation of Alkylglycerol Monooxygenase to Provide a Tetrahydrobiopterin-Tuneable Means to Overcome the Growth Arrest of Cultured Cells.

Animal cells in culture stop growing when reaching confluency. In production processes involving cultured cells, overcoming this growth stop may increase cellular yield and thus productivity. It has been demonstrated in Madin-Darby Canine Kidney (MDCK) cells, that the concentration of alkylglycerols increased up to 20 fold during the growth of MDCK cell cultures to a confluent density, thus inhibiting protein kinase C and proliferation (Warne, T. R. et al., J Biol Chem 270(19):11147-54 (1995)). Upregulation/overexpression of alkylglycerol monooxygenase is expected to lead to degradation of these lipids, to lower the concentration of accumulated alkylglycerols, to thereby reverse the alkylglycerol-mediated the inhibition of protein kinase C and hence to overcome the growth arrest. The enzymatic activity of alkylgylcerol monooxygenase in cultivated, intact Chinese hamster ovary cells is largely dependent on the amount of tetrahydrobiopterin supplied to the cells via the precursor drug sepiapterin. Thus, depending on the endogenous tetrahydrobiopterin production of the individual cell, the alleviation of the growth arrest is expected to be tuneable by tetrahydrobiopterin and/or by sepiapterin added to the culture medium.

### Ether-Lipid Degradation Induced Protein Kinase C Activation to Treat Alzheimer's Disease.

Degradation of alkylglycerols is expected to stimulate protein kinase C by removal of inhibitory alkylgylcerols, as outlined in the example of cell proliferation control above. Protein kinase C (PKC) signaling is critical for the non-toxic degradation of amyloid precursor protein (APP) and inhibition of GSK3beta, which controls phosphorylation of tau protein in Alzheimer's disease. Thus the misregulation of PKC signaling could contribute to the origins of Alzheimer's disease (Khan, T. K. et al., Neurobiol Dis. 34(2):332-9. (2009)). Modulation of PKC by degradation of inhibitory alkylglycerols thus has the potential to ameliorate both the neurodegeneration and the recent memory loss associated with Alzheimer's disease.

The pharmaceutical composition will be formulated and dosed in a fashion consistent with good medical practice, taking into account the clinical condition of the individual patient, the site of delivery of the pharmaceutical composition, the method of administration, the scheduling of administration, and other factors known to practitioners. The “effective amount” of the pharmaceutical composition for purposes herein is thus determined by such considerations.

The skilled person knows that the effective amount of pharmaceutical composition administered to an individual will, inter alia, depend on the nature of the compound. For example, if said compound is a (poly)peptide or protein the total pharmaceutically effective amount of pharmaceutical composition administered parenterally per dose will be in the range of about 1 μg protein/kg/day to 10 mg protein/kg/day of patient body weight, although, as noted above, this will be subject to therapeutic discretion. More preferably, this dose is at least 0.01 mg protein/kg/day, and most preferably for humans between about 0.01 and 1 mg protein/kg/day. If given continuously, the pharmaceutical composition is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect. The particular amounts may be determined by conventional tests which are well known to the person skilled in the art.

Pharmaceutical compositions of the invention may be administered orally, parenterally, intracisternally, intraperitoneally, topically (as by powders, ointments, drops or transdermal patch), bucally, or as an oral or nasal spray.

Pharmaceutical compositions of the invention preferably comprise a pharmaceutically acceptable carrier. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

The pharmaceutical composition is also suitably administered by sustained release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Sustained release pharmaceutical composition also include liposomally entrapped compound. Liposomes containing the pharmaceutical composition are prepared by methods known per se: DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal therapy.

For parenteral administration, the pharmaceutical composition is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation.

Generally, the formulations are prepared by contacting the components of the pharmaceutical composition uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non aqueous vehicles such as fixed oils and ethyl oleate are also useful herein, as well as liposomes. The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) (poly)peptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The components of the pharmaceutical composition to be used for therapeutic administration must be sterile. Sterility is readily accomplished by filtration through sterile filtration membranes (e.g., 0.2 micron membranes). Therapeutic components of the pharmaceutical composition generally are placed into a container having a sterile access port, for example, an intravenous solution bag or vial having a stopper pierceable by a hypodermic injection needle.

The components of the pharmaceutical composition ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous solution, and the resulting mixture is lyophilized. The infusion solution is prepared by reconstituting the lyophilized compound(s) using bacteriostatic Water-for-Injection.

The term “medical intervention” as herein described, refers to any examination, treatment, or other act having preventive, diagnostic therapeutic or rehabilitative aims and which is carried out by a physician or other health care provider (WHO).

The compounds of the present invention (polypeptides and fragments thereof, polynucleotides, vectors, host cells, antibodies etc.) may also be comprised in a diagnostic composition.

The term “diagnostic composition” as herein described, refers to one of the aforementioned compounds which are prepared to be used for diagnostic purposes. It is to be understood that depending on the nature of the diagnostic agent, i.e. depending on whether a polynucleotide, a polypeptide (or a fragment thereof), an antibody or an oligonucleotide is used, the diagnostic composition may comprise additional agents such as agents which allow hybridization, antibody binding or detection. Such additional agents are well known to those skilled in the art.

The term “diagnosis” as used herein means identification of pathological condition and features. For the purpose of the invention, a diagnosis is to identify a condition wherein a decrease or an increase in the activity of a functional alkylglycerol monooxygenase is expected to have various medical implications as outlined in more detail below. As explained in the following, a decrease in the activity of a functional alkylglycerol monooxygenase is expected to have medical implications, i.e., e.g., to have an antiproliferative/antitumor effect, to counteract hypertension, to restore male fertility, and to protect the eye from cateract. Furthermore, as also explained in the following, an increase in the activity of a functional alkylglycerol monooxygenase is expected to have medical implications, i.e., e.g., to restore male infertility and to ameliorate both the neurodegeneration and the recent memory loss associated with Alzheimer's disease.

In a particular embodiment, the present invention relates to a pharmaceutical composition comprising an antagonist/inhibitor of alkylglycerol monooxygenase (TMEM195; glyceryl ether monooxygenase; EC 1.14.16.5) as defined above.

The term “antagonist” or “inhibitor” as used herein is known in the art and relates to a compound/substance capable of fully or partially preventing or reducing the physiologic activity of (a) specific protein(s). In the context of the present invention said antagonist, therefore, may prevent or reduce or inhibit or inactivate the physiological activity of a protein such as alkylglycerol monooxygenase TMEM195 upon binding of said compound/substance to said protein. Binding of an “antagonist/inhibitor” to a given protein, e.g. alkylglycerol monooxygenase TMEM195, may compete with or prevent the binding of an endogenous activating molecules binding to said protein. As used herein, accordingly, the term “antagonist” also encompasses competitive antagonists, (reversible) non-competitive antagonists or irreversible antagonist, as described, inter alia, in Mutschler, “Arzneimittelwirkungen” (1986), Wissenschaftliche Verlagsgesellschaft mbH, Stuttgart, Germany. In addition thereto, however, an “antagonist” or “inhibitor” of alkylglycerol monooxygenase TMEM195 in the context of the present invention may also be capable of preventing the function of a given protein, such as alkylglycerol monooxygenase TMEM195, by preventing/reducing the expression of the nucleic acid molecule encoding for said protein. Thus, an antagonist/inhibitor of alkylglycerol monooxygenase TMEM195 may lead to a decreased expression level of alkylglycerol monooxygenase TMEM195 (e.g. decreased level of alkylglycerol monooxygenase TMEM195 mRNA, alkylglycerol monooxygenase TMEM195 protein) which is reflected in a decreased activity of alkylglycerol monooxygenase TMEM195. This decreased activity can be measured/detected by the herein described methods. An inhibitor of alkylglycerol monooxygenase TMEM195 in the context of the present invention, accordingly, may also encompass transcriptional repressors of alkylglycerol monooxygenase TMEM195 expression that are capable of reducing alkylglycerol monooxygenase TMEM195 function. As described herein below in detail, the decreased expression and/or activity of alkylglycerol monooxygenase TMEM195 by an antagonist/inhibitor of alkylglycerol monooxygenase TMEM195 leads to a decreased activity (and/or expression) of alkylglycerol monooxygenase TMEM195, thereby decreasing functional alkylglycerol monooxygenase activity.

Without being bound by theory, it is believed that a decrease or an increase in the activity of a functional alkylglycerol monooxygenase is expected to have various medical implications.

As explained in the following, a decrease in the activity of a functional alkylglycerol monooxygenase is expected to have medical implications, i.e., e.g., to have an antiproliferative/antitumor effect, to counteract hypertension, to restore male fertility, and to protect the eye from cateract. Furthermore, as also explained in the following, an increase in the activity of a functional alkylglycerol monooxygenase is expected to have medical implications, i.e., e.g., to restore male infertility and to ameliorate both the neurodegeneration and the recent memory loss associated with Alzheimer's disease.

The metabolism of ether lipids has not been studied in great detail yet. Limited information on physiological roles of ether lipids originates from observations on knockout mice (23). Like acyl-glycerols, ether lipids occur in many subspecies with varying lipid chains (with or without double bonds) attached to the glycerol backbone by an ether linkage. In addition, many lipids found in the body are alkyl acyl glycerols, with one alkyl and one acyl residue attached to glycerol in the 10 and 20 position (Yang, K. et al., PLoS One 2(12):e1368 (2007)). Substrates for alkylglycerol monooxygenase need a free hydroxy function in position 2 of the glycerol backbone, and a saturated bond (i.e. no double bond as in plasmalogens, which are no substrates) adjacent to the ether oxygen. Thus, to become substrates of alkylglycerol monooxygenase, alkyl acyl lipids have to be cleaved by phospholipase A2 type enzymes to the respective lyso lipids. All of the speculations listed below rely on the (reasonable) assumption, that inhibition of the degradation of 10 alkyl glycerol derivatives will modify the in vivo concentration of these compounds, and thereby alter their availability for biosynthesis and hence also alter the concentrations of biosynthetic products formed from these.

Consequently, in particular, without being bound by theory, it is believed that inhibitors/antagonists of alkylglycerol monooxygenase TMEM195 lead to a decrease in the activity of a functional alkylglycerol monooxygenase wherein such a decrease is expected to have medical implications, i.e., e.g., to have an antiproliferative/antitumor effect, to counteract hypertension, to restore male fertility, and to protect the eye from cateract:

### Antiproliferative/Antitumor Action.

Cell proliferation is stimulation by proteinkinase C, which is activated by diacylglycerols and inhibited by alkylglycerols. Endogenously accumulating alkylglycerols are responsible for the growth arrest of cells when reaching confluency (Warne, T. R. et al., J Biol Chem 270(19):11147-54 (1995)) Inhibition/downregulation of alkylgylcerol monooxygenase and the expected rise in intracellular alkylglycerols will therefore have an antiproliferative effect on its one and enhance the action of e.g. alkyllysophospholid anticancer drugs (Glasser, L. et al., Exp Hematol. 24(2):253-7 (1996)).

### Hypertension.

Lowered concentrations of etherlipids have been found to be asscociated with hypertension independent of obesity and insuline resistance (48). Inhibition/downregulation of alkylglycerol monooxygenase could correct this to normal, and thereby counteract hypertension, provided that the association of lowered ether lipids and hypertension is a functional one.

Protection of the Eye from Cataract.

The eye is a tissue with exceptionally high content of etherlipids. If the biosynthesis is impaired as in knockout mice, the frequency of cataract formation is increased (23). Inhibition/downregulation of alkylglycerol monooxygenase thus could increase the concentrations of the endogenous etherlipids and thereby protect the eye from cataract.

In contrast, without being bound by theory, it is believed that agonists/activators of alkylglycerol monooxygenase TMEM195 lead to an increase in the activity of a functional alkylglycerol monooxygenase wherein such an increase is expected to have medical implications, i.e., e.g., to ameliorate both the neurodegeneration and the recent memory loss associated with Alzheimer's disease, and to induce male infertility.

### Testis/Male Infertility.

Testes contain a complex ether lipid, the called seminolipid (23). Male mice deficient in the biosynthesis of ether lipids are infertile. Infertility of men is only understood in a minor part of the cases (O'Flynn O'Brien, K. L. et al., Fertil Steril 93(1):1-12 (2010)). Testis is an organ with high alkylglycerol monooxygenase activity. In cases where a partial inhibition of alkylglycerol biosynthesis may cause male infertility due to defective biosynthesis of seminolipid or its precursors, dimishing the activity of alkylglycerol has the potential to restore fertility. On the contrary, increasing alkylglycerol monooxgyenase activity in testis is expected to elicit male infertility by degrading etherlipid precursors of seminolipid, thereby causing male infertility comparable to mice deficient in etherlipid biosynthesis (23).

### Overexpression/Upregulation of Alkylglycerol Monooxygenase to Provide a Tetrahydrobiopterin-Tuneable Means to Overcome the Growth Arrest of Cultured Cells.

Animal cells in culture stop growing when reaching confluency. In production processes involving cultured cells, overcoming this growth stop may increase cellular yield and thus productivity. It has been demonstrated in Madin-Darby Canine Kidney (MDCK) cells, that the concentration of alkylglycerols increased up to 20 fold during the growth of MDCK cell cultures to a confluent density, thus inhibiting protein kinase C and proliferation (Warne, T. R. et al., J Biol Chem 270(19):11147-54 (1995)). Upregulation/overexpression of alkylglycerol monooxygenase is expected to lead to degradation of these lipids, to lower the concentration of accumulated alkylglycerols, to thereby reverse the alkylglycerol-mediated the inhibition of protein kinase C and hence to overcome the growth arrest. The enzymatic activity of alkylgylcerol monooxygenase in cultivated, intact Chinese hamster ovary cells is largely dependent on the amount of tetrahydrobiopterin supplied to the cells via the precursor drug sepiapterin. Thus, depending on the endogenous tetrahydrobiopterin production of the individual cell, the alleviation of the growth arrest is expected to be tuneable by tetrahydrobiopterin and/or by sepiapterin added to the culture medium.

### Ether-Lipid Degradation Induced Protein Kinase C Activation to Treat Alzheimer's Disease.

Degradation of alkylglycerols is expected to stimulated protein kinase C by removal of inhibitory alkylgylcerols, as outlined in the example of cell proliferation control above. Protien kinase C (PKC) signaling is critical for the non-toxic degradation of amyloid precursor protein (APP) and inhibition of GSK3beta, which controls phosphorylation of tau protein in Alzheimer's disease. Thus the misregulation of PKC signaling could contribute to the origins of Alzheimer's disease (Khan, T. K. et al., Neurobiol Dis. 34(2):332-9. (2009)). Modulation of PKC by degradation of inhibitory alkylglycerols thus has the potential to ameliorate both the neurodegeneration and the recent memory loss associated with Alzheimer's disease.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a subject and includes: (a) preventing cancer, hypertesion, cateract or Alzheimer's disease from occurring in a subject which may be predisposed to the disease; (b) inhibiting the disease, i.e. arresting its development; or (c) relieving the disease, i.e. causing regression of the disease.

A “patient” or “subject” for the purposes of the present invention includes both humans and other animals, particularly mammals, and other organisms. Thus, the methods are applicable to both human therapy and veterinary applications. In the preferred embodiment the patient is a mammal, and in the most preferred embodiment the patient is human.

The compounds capable of reducing alkylglycerol monooxygenase TMEM195 function or (a) fragment(s) thereof, are expected to be very beneficial as agents in pharmaceutical settings disclosed herein and to be used for medical purposes, in particular, in the treatment of cancer, hypertension, male infertility and cateract. Said antagonist/inhibitor of alkylglycerol monooxygenase TMEM195 may be selected from the group consisting of alkylglycerol monooxygenase TMEM195 inhibitory peptide, small binding molecules, RNAi, anti-alkylglycerol monooxygenase TMEM195 antisense molecules, intracellular binding-partners of alkylglycerol monooxygenase TMEM195, aptamers or intramers specifically directed against alkylglycerol monooxygenase TMEM195.

Compounds which may function as specific an “antagonist” or “inhibitor” of alkylglycerol monooxygenase TMEM 195 may comprise small binding molecules such as small (organic) compounds or ligands for alkylglycerol monooxygenase TMEM195. The term “small molecule” in the context of drug discovery is known in the art and relates to medical compounds having a molecular weight of less than 2,500 Daltons, preferably less than 1,000 Daltons, more preferably between 50 and 350 daltons. (Small) binding molecules comprise natural as well as synthetic compounds. The term “compound” in context of this invention comprises single substances or a plurality of substances. Said compound/binding molecules may be comprised in, for example, samples, e.g., cell extracts from, e.g., plants, animals or microorganisms. Furthermore, said compound(s) may be known in the art but hitherto not known to be capable of (negatively) influencing the activity alkylglycerol monooxygenase TMEM195 or not known to be capable of influencing the expression of the nucleic acid molecule encoding for alkylglycerol monooxygenase TMEM195, respectively. The plurality of compounds may be, e.g., added to a sample in vitro, to the culture medium or injected into the cell.

Yet it is also envisaged in the context of the present invention that compounds including, inter alia, peptides, proteins, nucleic acids including cDNA expression libraries, small organic compounds, ligands, PNAs and the like can be used as an antagonist of alkylglycerol monooxygenase TMEM195 function. Said compounds can also be functional derivatives or analogues. Methods for the preparation of chemical derivatives and analogues are well known to those skilled in the art and are described in, for example, Beilstein, “Handbook of Organic Chemistry”, Springer Edition New York, or in “Organic Synthesis”, Wiley, New York. Furthermore, said derivatives and analogues can be tested for their effects, i.e. their antagonistic effects of alkylglycerol monooxygenase TMEM195 function according to methods known in the art. Furthermore, peptidomimetics and/or computer aided design of appropriate antagonists or inhibitors of alkylglycerol monooxygenase TMEM195 can be used. Appropriate computer systems for the computer aided design of, e.g., proteins and peptides are described in the prior art, for example, in Berry (1994) Biochem. Soc. Trans. 22:1033-1036; Wodak (1987), Ann. N.Y. Acad. Sci. 501:1-13; Pabo (1986), Biochemistry 25:5987-5991. The results obtained from the above-described computer analysis can be used in combination with the method of the invention for, e.g., optimizing known compounds, substances or molecules. Appropriate compounds can also be identified by the synthesis of peptidomimetic combinatorial libraries through successive chemical modification and testing the resulting compounds, e.g., according to the methods described herein. Methods for the generation and use of peptidomimetic combinatorial libraries are described in the prior art, for example in Ostresh (1996) Methods in Enzymology 267:220-234 and Dorner (1996) Bioorg. Med. Chem. 4:709-715. Furthermore, the three-dimensional and/or crystallographic structure of antagonists of alkylglycerol monooxygenase TMEM195 can be used for the design of (peptidomimetic) antagonists of alkylglycerol monooxygenase TMEM 195.

The RNAi-approach is also envisaged in context of this invention for use in the preparation of a pharmaceutical composition for the treatment of diseases/disorders related to cancer, hypertension, male infertility or cateract.

The term “RNA interference” or “inhibiting RNA” (RNAi/iRNA) describes the use of double-stranded RNA to target specific mRNAs for degradation, thereby silencing their expression. Preferred inhibiting RNA molecules may be selected from the group consisting of double-stranded RNA (dsRNA), RNAi, siRNA, shRNA and stRNA. dsRNA matching a gene sequence is synthesized in vitro and introduced into a cell. The dsRNA may also be introduced into a cell in form of a vector expressing a target gene sequence in sense and antisense orientation, for example in form of a hairpin mRNA. The sense and antisense sequences may also be expressed from separate vectors, whereby the individual antisense and sense molecules form double-stranded RNA upon their expression. It is known in the art that in some occasions the expression of a sequence in sense orientation or even of a promoter sequence suffices to give rise to dsRNA and subsequently to siRNA due to internal amplification mechanisms in a cell. Accordingly, all means and methods which result in a decrease in activity (which may be reflected in a lower expression of alkylglycerol monooxygenase TMEM195), in particular by taking advantage of alkylglycerol monooxygenase TMEM195-specific siRNAs (i.e. siRNAs that target specifically alkylglycerol monooxygenase TMEM195 mRNA or a functional fragment thereof) are to be used in accordance with the present invention. For example sense constructs, antisense constructs, hairpin constructs, sense and antisense molecules and combinations thereof can be used to generate/introduce these siRNAs. The dsRNA feeds into a natural, but only partially understood process including the highly conserved nuclease dicer which cleaves dsRNA precursor molecules into short interfering RNAs (siRNAs). The generation and preparation of siRNA(s) as well as the method for inhibiting the expression of a target gene is, inter alia, described in WO 02/055693, Wei (2000) Dev. Biol. 15:239-255; La Count (2000) Biochem. Paras. 111:67-76; Baker (2000) Curr. Biol. 10:1071-1074; Svoboda (2000) Development 127:4147-4156 or Marie (2000) Curr. Biol. 10:289-292. These siRNAs built then the sequence specific part of an RNA-induced silencing complex (RISC), a multicomplex nuclease that destroys messenger RNAs homologous to the silencing trigger). Elbashir (2001) EMBO J. 20:6877-6888 showed that duplexes of 21 nucleotide RNAs may be used in cell culture to interfere with gene expression in mammalian cells. It is already known that RNAi is mediated very efficiently by siRNA in mammalian cells but the generation of stable cell lines or non-human transgenic animals was limited. However, new generations of vectors may be employed in order to stably express, e.g. short hairpin RNAs (shRNAs). Stable expression of siRNAs in Mammalian Cells is inter alia shown in Brummelkamp (2002) Science 296:550-553. Also Paul (2002) Nat. Biotechnol. 20:505-508 documented the effective expression of small interfering RNA in human cells. RNA interference by expression of short-interfering RNAs and hairpin RNAs in mammalian cells was also shown by Yu (2002) PNAS 99:6047-6052. The shRNA approach for gene silencing is well known in the art and may comprise the use of st (small temporal) RNAs; see, inter alia, Paddison (2002) Genes Dev. 16:948-958. These approaches may be vector-based, e.g. the pSUPER vector, or RNA polIII vectors may be employed as illustrated, inter alia, in Yu (2002), loc. cit.; Miyagishi (2002), loc. cit. or Brummelkamp (2002), loc. cit. It is envisaged that the regulatory sequences of the present invention are used in similar fashion as the systems based on pSUPER or RNA polIII vectors.

Methods to deduce and construct siRNAs are known in the art and are described in Elbashir (2002) Methods 26:199-213, at the internet web sites of commercial vendors of siRNA, e.g. Qiagen GmbH (https://wwwl.qiagen.com/GeneGlobe/Default.aspx); Dharmacon (www.dharmacon.com); Xeragon Inc. (http://www.dharmacon.com/Default.aspx), and Ambion (www.ambion.com), or at the web site of the research group of Tom Tuschl (http://www.rockefeller.edu/labheads/tuschl/sirna.html). In addition, programs are available online to deduce siRNAs from a given mRNA sequence (e.g. http://www.ambion.com/techlib/misc/siRNA_finder.html or http://katandin.cshl.org:9331/RNAi/html/rnai.html). Uridine residues in the 2-nt 3′ overhang can be replaced by 2′ deoxythymidine without loss of activity, which significantly reduces costs of RNA synthesis and may also enhance resistance of siRNA duplexes when applied to mammalian cells (Elbashir (2001) loc. cit). The siRNAs may also be sythesized enzymatically using T7 or other RNA polymerases (Donze (2002) Nucleic Acids Res 30:e46). Short RNA duplexes that mediate effective RNA interference (esiRNA) may also be produced by hydrolysis with Escherichia coli RNase 111 (Yang (2002) PNAS 99:9942-9947). Furthermore, expression vectors have been developed to express double stranded siRNAs connected by small hairpin RNA loops in eukaryotic cells (e.g. (Brummelkamp (2002) Science 296:550-553). All of these constructs may be developed with the help of the programs named above. In addition, commercially available sequence prediction tools incorporated in sequence analysis programs or sold separately, e.g. the siRNA Design Tool offered by www.oligoEngine.com (Seattle, Wash.) may be used for siRNA sequence prediction.

Accordingly, specific interfering RNAs can be used in accordance with the present invention as antagonists (inhibitors) of alkylglycerol monooxygenase TMEM195 (expression and/or function). These siRNAs are formed by an antisense and a sense strand, whereby the antisense/sense strand preferably comprises at least 10, more preferably at least 12, more preferably at least 14, more preferably at least 16, more preferably at least 18, more preferably at least 19, 20, 21 or 22 nucleotides.

As mentioned above, methods for preparing siRNAs to be used in accordance with the present invention are well known in the art. Based on the teaching provided herein, a skilled person in the art is easily in the position not only to prepare such siRNAs but also to assess whether a siRNA is capable of antagonizing/inhibiting alkylglycerol monooxygenase TMEM195. It is envisaged herein that the above described siRNAs lead to a degradation of alkylglycerol monooxygenase TMEM195 mRNA and thus to a decreased protein level of alkylglycerol monooxygenase TMEM 195.

In other words, siRNAs lead to a pronounced decrease in mRNA and/or protein levels of alkylglycerol monooxygenase TMEM195 (i.e. to a reduced expression of alkylglycerol monooxygenase TMEM195). This decrease in expression may be reflected in a decreased activity of alkylglycerol monooxygenase TMEM195. For example, alkylglycerol monooxygenase TMEM195-specific siRNAs may lead to a decreased capacity of alkylglycerol monooxygenase TMEM195 and to inhibit alkylglycerol monooxygenase TMEM195 activity. Hence, the use of potent antagonists/inhibitors of alkylglycerol monooxygenase TMEM195 (such as the herein described siRNAs) will lead to a lower alkylglycerol monooxygenase TMEM195 activity.

As used herein the term “small interfering RNA” (siRNA), sometimes known as short interfering RNA or silencing RNA, refers to a class of generally short and double-stranded RNA molecules that play a variety of roles in biology and, to an increasing extent, in treatment of a variety of diseases and conditions. As mentioned above, siRNAs are involved in the RNA interference (RNAi) pathway where the siRNA interferes with the expression of a specific gene (see, e.g. Zamore Nat Struct Biol 2001, 8(9):746-50; Tuschl T. CHEMBIOCHEM. 2001, 2:239-245; Scherr and Eder, Cell Cycle. 2007 February; 6(4):444-9; Leung and Whittaker, Pharmacol Ther. 2005 August; 107(2):222-39; de Fougerolles et al., Nat. Rev. Drug Discov. 2007, 6: 443-453).

Such siRNAs are generally 18-27 nt long, generally comprising a short (usually 19-21-nt) double-strand of RNA (dsRNA) with or without 2-nt 3′ overhangs on either end. Each strand can have a 5′ phosphate group and a 3′ hydroxyl (—OH) group or the phosphate group can be absent on one or both strands. This structure is the result of processing by dicer, an enzyme that converts either long dsRNAs or small hairpin RNAs into siRNAs.

siRNAs can also be exogenously (artificially) introduced into cells by various transfection methods to bring about the specific knockdown of a gene of interest. In this context, other structures than those described above are also envisaged, provided they are capable of interfering with gene expression. Preferably, the double-stranded part has a length of about 12 to about 50 base pairs, more preferably 16 to 30, more preferably 18 to 25, more preferably 19 to 21 in length. Most preferably, the double-stranded part has a length of 19 base pairs. The siRNA of the invention may either have overhanging sequences of up to 10 bases, preferably not more than 5 bases in length at either end or at one end, or may be blunt-ended. Also preferred is that the complementarity to the target gene extends over the entire length of the double-stranded part. The region which is complementary to the target gene is at least 12 bases, preferably at least 15, 16, 17, 18, 19, 20, 21, 22, 23 or more bases in length. The siRNA of the invention may be fully complementary to the target gene. Alternatively, the siRNA may comprise up to 5%, 10%, 20% or 30% mismatches to the target gene. Furthermore, siRNAs and also antisense RNAs can be chemically modified e.g. on the backbone including the sugar residues. Preferred modifications of the siRNA molecules of the invention include linkers connecting the two strands of the siRNA molecule. Chemical modifications serve inter alia to improve the pharmacological properties of siRNAs and antisense RNAs such as in vivo stability and/or delivery to the target site within an organism. The skilled person is aware of such modified siRNAs as well as of means and methods of obtaining them, see, for example, Zhang et al., Curr Top Med. Chem. 2006; 6(9):893-900; Manoharan, Curr Opin Chem. Biol. 2004 December; 8(6):570-9.

Essentially any gene of which the sequence is known can thus be targeted based on sequence complementarity with an appropriately tailored siRNA. This has made siRNAs an important tool for gene function and drug target validation studies as well as for therapeutic intervention which is envisaged here. The siRNAs disclosed herein are capable of reducing or blocking the expression of alkylglycerol monooxygenase TMEM195.

In a further aspect, it is envisaged that antisense molecules inhibit the expression or function of alkylglycerol monooxygenase TMEM195, in particular of human alkylglycerol monooxygenase TMEM195 and interact with alkylglycerol monooxygenase TMEM195 as expressed by the coding regions, mRNAs/cDNAs as defined herein above as well as with alkylglycerol monooxygenase TMEM195 as expressed by isoforms and variants of said alkylglycerol monooxygenase TMEM195. Said isoforms or variants may, inter alia, comprise allelic variants or splice variants. Furthermore, it is also envisaged that the antisense molecules to be used in accordance with the present invention against alkylglycerol monooxygenase TMEM 195 expression or function interfere specifically with regulatory sequences of alkylglycerol monooxygenase TMEM195 as defined herein below.

The term “variant” means in this context that the alkylglycerol monooxygenase TMEM195 nucleotide sequence and the encoded alkylglycerol monooxygenase TMEM195 amino acid sequence, respectively, differs from the distinct sequences available under the above-identified GenBank Accession numbers, by mutations, e.g. deletion, additions, substitutions, inversions etc.

Therefore, the antisense-molecule to be employed in accordance with the present invention specifically interacts with/hybridizes to one or more nucleic acid molecules encoding alkylglycerol monooxygenase TMEM195. Preferably said nucleic acid molecule is RNA, i.e. pre m-RNA or mRNA. The term “specifically interacts with/hybridizes to one or more nucleic acid molecules encoding alkylglycerol monooxygenase TMEM195” relates, in context of this invention, to antisense molecules which are capable of interfering with the expression of alkylglycerol monooxygenase TMEM195. Yet, highly mutated anti-alkylglycerol monooxygenase TMEM 195 antisense constructs, which are not capable of hybridizing to or specifically interacting with alkylglycerol monooxygenase TMEM195-coding nucleic acid molecules are not to be employed in the context of the present invention. The person skilled in the art can easily deduce whether an antisense construct specifically interacts with/hybridizes to alkylglycerol monooxygenase TMEM195 encoding sequences. These tests comprise, but are not limited to hybridization assays, RNAse protection assays, Northern Blots, North-western blots, nuclear magnetic resonance and fluorescence binding assays, dot blots, micro- and macroarrays and quantitative PCR. In addition, such a screening may not be restricted to alkylglycerol monooxygenase TMEM195 mRNA molecules, but may also include alkylglycerol monooxygenase TMEM195 mRNA/protein (RNP) complexes (Hermann (2000) Angew Chem Int Ed Engl 39:1890-1904; DeJong (2002) Curr Trop Med Chem 2:289-302). Furthermore, functional tests including Western blots, immunohistochemistry, immunoprecipitation assay, and bioassays based on alkylglycerol monooxygenase TMEM195-responsive promoters are envisaged for testing whether a particular antisense construct is capable of specifically interacting with/hybridizing to the alkylglycerol monooxygenase TMEM195 encoding nucleic acid molecules.

The term “antisense-molecule” as used herein comprises in particular antisense oligonucleotides. Said antisense oligonucleotides may also comprise modified nucleotides as well as modified internucleoside-linkage, as, inter alia, described in U.S. Pat. No. 6,159,697.

Most preferably, the antisense oligonucleotides of the present invention comprise at least 8, more preferably at least 10, more preferably at least 12, more preferably at least 14, more preferably at least 16 nucleotides. The deduction as well as the preparation of antisense molecules is very well known in the art. The deduction of antisense molecules is, inter alia, described in Smith, 2000. Usual methods are “gene walking”, Rnase H mapping, RNase L mapping (Leaman (1999) Meth Enzymol 18:252-265), combinatorial oligonucleotide arrays on solid support, determination of secondary structure analysis by computational methods (Walton (2000) Biotechnol Bioeng, 65:1-9), aptamer oligonucleotides targeted to structured nucleic acids (aptastruc), thetered oligonucleotide probes, foldback triplex-forming oligonucleotides (FTFOs) (Kandimalla (1994) Gene 149:115-121) and selection of sequences with minimized non-specific binding (Han (1994) Antisense Res Dev 4:53-65).

Preferably, the antisense molecules of the present invention are stabilized against degradation. Such stabilization methods are known in the art and, inter alia, described in U.S. Pat. No. 6,159,697. Further methods described to protect oligonucleotides from degradation include oligonucleotides bridged by linkers (Vorobjev (2001) Antisense Nucleic Acid Drug Dev, 11:77-85), minimally modified molecules according to cell nuclease activity (Samani (2001) Antisense Nucleic Acid Drug Dev, 11:129-136), 2′O-DMAOE oligonucleotides (Prakash (2001) Nucleosides Nucleotides Nucleic Acids 20:829-832), 3′5′-Dipeptidyl oligonucleotides (Schwope (1999) J Org Chem 64:4749-4761), 3′ methylene thymidine and 5-methyluridine/cytidine h-phosphonates and phosphonamidites (An (2001) J Org Chem, 66:2789-2801), as well as anionic liposome (De Oliveira (2000) Life Sci 67:1625-1637) or ionizable aminolipid (Semple (2001) Biochim Biophys Acta, 10:152-166) encapsulation.

In addition thereto, the antagonist/inhibitor of alkylglycerol monooxygenase TMEM195 expression or function may also comprise intracellular binding partners of alkylglycerol monooxygenase TMEM195. As used herein, the term “intracellular binding partner” relates to intracellular molecules capable of preventing or reducing alkylglycerol monooxygenase TMEM195 activity. Such intracellular binding partners of alkylglycerol monooxygenase TMEM195, inter alia, may relate to endogenous inhibitor/repressor proteins of alkylglycerol monooxygenase TMEM195. In another embodiment of the invention the intracellular binding partner is an intracellular antibody. Intracellular antibodies are known in the art and can be used to modulate or inhibit the functional activity of the target molecule. This therapeutic approach is based on intracellular expression of recombinant antibody fragments, either Fab or single chain Fv, targeted to the desired cell compartment using appropriate targeting sequences (Teillaud (1999) Pathol Biol 47:771-775).

As mentioned herein above, the antagonist/inhibitor of alkylglycerol monooxygenase TMEM195 expression or function may also comprise an aptamer. In the context of the present invention, the term “aptamer” comprises nucleic acids such as RNA, ssDNA (ss=single stranded), modified RNA, modified ssDNA or PNAs which bind a plurality of target sequences having a high specificity and affinity. Aptamers are well known in the art and, inter alia, described in Famulok (1998) Curr. Op. Chem. Biol. 2:320-327. The preparation of aptamers is well known in the art and may involve, inter alia, the use of combinatorial RNA libraries to identify binding sites (Gold (1995) Ann. Rev. Biochem. 64:763-797).

Accordingly, aptamers are oligonucleotides derived from an in vitro evolution process called SELEX (systematic evolution of ligands by exponential enrichment). Pools of randomized RNA or single stranded DNA sequences are selected against certain targets. The sequences of tighter binding with the targets are isolated and amplified. The selection is repeated using the enriched pool derived from the first round selection. Several rounds of this process lead to winning sequences that are called “aptamers”. Aptamers have been evolved to bind proteins which are associated with a number of disease states. Using this method, many powerful antagonists of such proteins can be found. In order for these antagonists to work in animal models of disease and in humans, it is normally necessary to modify the aptamers. First of all, sugar modifications of nucleoside triphosphates are necessary to render the resulting aptamers resistant to nucleases found in serum. Changing the 2′OH groups of ribose to 2′F or 2′NH2 groups yields aptamers which are long lived in blood. The relatively low molecular weight of aptamers (8000-12000) leads to rapid clearance from the blood. Aptamers can be kept in the circulation from hours to days by conjugating them to higher molecular weight vehicles. When modified, conjugated aptamers are injected into animals, they inhibit physiological functions known to be associated with their target proteins. Aptamers may be applied systemically in animals and humans to treat organ specific diseases (Ostendorf (2001) J Am Soc Nephrol. 12:909-918). The first aptamer that has proceeded to phase I clinical studies is NX-1838, an injectable angiogenesis inhibitor that can be potentially used to treat macular degeneration-induced blindness. (Sun (2000) Curr Opin Mol Ther 2:100-105). Cytoplasmatic expression of aptamers (“intramers”) may be used to bind intracellular targets (Blind (1999) PNAS 96:3606-3610; Mayer (2001) PNAS 98:4961-4965). Said intramers are also envisaged to be employed in context of this invention.

As used herein, the term “nucleic acid sequence” relates to the sequence of bases comprising purine- and pyrimidine bases which are comprised by nucleic acid molecules, whereby said bases represent the primary structure of a nucleic acid molecule. Nucleic acid sequences include DNA, cDNA, genomic DNA, RNA, synthetic forms and mixed polymers, both sense and antisense strands, or may contain non-natural or derivatized nucleotide bases, as will be readily appreciated by those skilled in the art. Preferably, the term “alkylglycerol monooxygenase TMEM195” when used in the context of expressing alkylglycerol monooxygenase TMEM 195 refers to the nucleic acid molecule encoding alkylglycerol monooxygenase TMEM 195 protein, or a functional fragment thereof. Exemplary nucleic acid sequences are known in the art and also disclosed herein.

As used herein, the term “polypeptide” relates to a peptide, a protein, or a polypeptide which encompasses amino acid chains of a given length, wherein the amino acid residues are linked by covalent peptide bonds. However, peptidomimetics of such proteins/polypeptides wherein amino acid(s) and/or peptide bond(s) have been replaced by functional analogs are also encompassed by the invention as well as other than the 20 gene-encoded amino acids, such as selenocysteine. Peptides, oligopeptides and proteins may be termed polypeptides. The terms polypeptide and protein are often used interchangeably herein. The term polypeptide also refers to, and does not exclude, modifications of the polypeptide, e.g., glycosylation, acetylation, phosphorylation and the like. Such modifications are well described in basic texts and in more detailed monographs, as well as in a voluminous research literature. Preferably, the term “alkylglycerol monooxygenase TMEM195”, particularly when used in context of “activity of alkylglycerol monooxygenase TMEM195”, refers to the protein/polypeptide having the specific alkylglycerol monooxygenase TMEM195 activity as disclosed herein.

As used herein, a “functional fragment” of a protein which displays a specific biological activity relates to fragments of said protein having a sufficient length to display said activity. Accordingly, a functional fragment of a protein showing e.g. a specific (enzymatic) activity may relate to a polypeptide which corresponds to a fragment of said protein which is still capable of showing said (enzymatic) activity. For example, a functional fragment of alkylglycerol monooxygenase TMEM195 in the context of the protein binding activity of alkylglycerol monooxygenase TMEM195 may correspond to the protein-binding domain of alkylglycerol monooxygenase TMEM195 as defined herein below. Methods for determining whether a certain fragment of a protein is a functional fragment are known in the art. For example, test for determining whether a fragment of alkylglycerol monooxygenase TMEM195 is still capable of binding a protein are described herein below. Preferably, a functional fragment of alkylglycerol monooxygenase TMEM195 has substantially the same biological activity as alkylglycerol monooxygenase TMEM195 itself. Furthermore, a person skilled in the art will be aware that the (biological) activity as described herein often correlates with the expression level, preferably the protein or mRNA level. The term “expression” as used herein refers to the expression of a nucleic acid molecule encoding a polypeptide/protein, whereas “activity” refers to the activity of said polypeptide/protein, which can be determined as outlined herein. The explanations given herein above and below in respect of the activity of “alkylglycerol monooxygenase TMEM195” also apply, mutatis mutandis, to (a) “functional fragment(s) of alkylglycerol monooxygenase TMEM195”. In other words, a “functional fragment of alkylglycerol monooxygenase TMEM195” has essentially the same activity as alkylglycerol monooxygenase TMEM195 as defined herein. Accordingly, also inhibitors/antagonists of functional fragments of alkylglycerol monooxygenase TMEM195 are disclosed and provided herein. As mentioned, methods/assays for determining the activity of “alkylglycerol monooxygenase TMEM195” and “functional fragment of alkylglycerol monooxygenase TMEM195” are well known in the art and also described herein above and below. Preferably, the functional fragment has at least 60%, more preferably at least 70%, 75%, 80%, 85%, 90% and even more preferably at least 95% or 99% of alkylglycerol monooxygenase TMEM195.

Alkylglycerol monooxygenase TMEM195 antagonists/inhibitors of alkylglycerol monooxygenase TMEM195 function may be deduced by methods in the art. Such methods are described herein and, inter alia, may comprise, but are not limited to methods where a collection of substances is tested for interaction with alkylglycerol monooxygenase TMEM195 or with (a) fragment(s) thereof and where substances which test positive for interaction in a corresponding readout system are further tested in vivo, in vitro or in silico for their inhibiting effects on alkylglycerol monooxygenase TMEM195 expression or function.

Said “test for alkylglycerol monooxygenase TMEM195 interaction” of the above described method may be carried out by specific immunological, molecular biological and/or biochemical assays which are well known in the art and which comprise, e.g., homogenous and heterogenous assays as described herein below. The natural endogenous ligand(s) of alkylglycerol monooxygenase TMEM195 remain(s) to be identified. Yet, alkylglycerol monooxygenase TMEM195 ligands capable of inhibiting alkylglycerol monooxygenase TMEM195 function may be identified by screening large compound libraries based on their capacity to interact with the alkylglycerol monooxygenase TMEM195 protein. In a preferred embodiment, such antagonists or inhibitors of alkylglycerol monooxygenase TMEM 195 function are capable of binding the protein binding domain of alkylglycerol monooxygenase TMEM195.

Besides molecules capable of binding to alkylglycerol monooxygenase TMEM195, antagonists or inhibitors of alkylglycerol monooxygenase TMEM195 function may be capable of preventing/reducing the expression of the nucleic acid molecule encoding the alkylglycerol monooxygenase TMEM195 protein. The skilled person is readily capable of identifying regulatory sequences (such as promoter sequences, enhancer sequences, replication origins and other regulatory elements) of alkylglycerol monooxygenase TMEM195 expression e.g. by using in silico gene prediction methods and experimental validation of functional sites (EInitski (2006) Genome Res 16:1455-64).

In a further aspect, the present invention relates to a pharmaceutical composition comprising the antagonist/inhibitor of alkylglycerol monooxygenase TMEM195 as described herein, optionally further comprising a pharmaceutical carrier. The (pharmaceutical) compositions of the invention may be in solid or liquid form and may be, inter alia, in a form of (a) powder(s), (a) tablet(s), (a) solution(s) or (an) aerosol(s). Furthermore, it is envisaged that the medicament of the invention might comprise further biologically active agents, depending on the intended use of the pharmaceutical composition.

Administration of the suitable (pharmaceutical) compositions may be effected by different ways, e.g., by parenteral, subcutaneous, intraperitoneal, topical, intrabronchial, intrapulmonary and intranasal administration and, if desired for local treatment, intralesional administration. Parenteral administrations include intraperitoneal, intramuscular, intradermal, subcutaneous intravenous or intraarterial administration. The compositions of the invention may also be administered directly to the target site, e.g., by biolistic delivery to an external or internal target site, like a specifically effected organ.

Examples of suitable pharmaceutical carriers, excipients and/or diluents are well known in the art and include phosphate buffered saline solutions, water, emulsions, such as oil/water emulsions, various types of wetting agents, sterile solutions etc. Compositions comprising such carriers can be formulated by well known conventional methods. Suitable carriers may comprise any material which, when combined with the biologically active protein of the invention, retains the biological activity of the comprised antagonist/inhibitor of alkylglycerol monooxygenase TMEM 195 (see Remington's Pharmaceutical Sciences (1980) 16th edition, Osol, A. Ed). Preparations for parenteral administration may include sterile aqueous or non-aqueous solutions, suspensions, and emulsions). Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles may include sodium chloride solution, Ringer's dextrose, dextrose and sodium chloride, lactated Ringer's, or fixed oils. Intravenous vehicles may include fluid and nutrient replenishes, electrolyte replenishers (such as those based on Ringer's dextrose), and the like. Preservatives and other additives may also be present including, for example, antimicrobials, anti-oxidants, chelating agents, and inert gases and the like. In addition, the pharmaceutical composition of the present invention might comprise proteinaceous carriers, like, e.g., serum albumin or immunoglobulin, preferably of human origin.

These pharmaceutical compositions can be administered to the subject at a suitable dose. The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently. Pharmaceutically active matter may be present in amounts between 1 μg and 20 mg/kg body weight per dose, e.g. between 0.1 mg to 10 mg/kg body weight, e.g. between 0.5 mg to 5 mg/kg body weight. If the regimen is a continuous infusion, it should also be in the range of 1 μg to 10 mg per kilogram of body weight per minute. Yet, doses below or above the indicated exemplary ranges also are envisioned, especially considering the aforementioned factors.

Furthermore, it is envisaged that the pharmaceutical composition of the invention might comprise further biologically active agents, depending on the intended use of the pharmaceutical composition. These further biologically active agents may be e.g. antibodies, antibody fragments, hormones, growth factors, enzymes, binding molecules, cytokines, chemokines, nucleic acid molecules and drugs.

Furthermore, the present invention relates to a screening method for assessing the activity of a candidate molecule suspected of being an antagonist/inhibitor of alkylglycerol monooxygenase TMEM195 which comprises the measurement of the activity of alkylglycerol monooxygenase TMEM 195.

Accordingly, screening methods for antagonists/inhibitors of alkylglycerol monooxygenase TMEM195 in cells, tissue and/or a non-human animal are provided. Also identification methods for antagonists of alkylglycerol monooxygenase TMEM195 are provided. These methods are highly useful in identifying/screening (a) candidate molecule(s) suspected of being inhibitors of alkylglycerol monooxygenase TMEM195 activity. Potent inhibitors identified/screened by these methods can be used in the medical intervention of a condition wherein a decrease in the activity of a functional alkylglycerol monooxygenase is expected to have medical implications as outlined above i.e., e.g., to have an antiproliferative effect, to counteract hypertension, restore male fertility or to ameliorate or prevent cateract. In accordance with the present invention, a candidate molecule that may be suspected of being an antagonist of alkylglycerol monooxygenase TMEM195 can, in principle, be obtained from any source as defined herein. The candidate molecule(s) may be (a) naturally occurring substance(s) or (a) substance(s) produced by a transgenic organism and optionally purified to a certain degree and/or further modified as described herein. Practically, the candidate molecule may be taken from a compound library as they are routinely applied for screening processes.

Accordingly, the present invention relates to method for assessing the activity of a candidate molecule suspected of being an antagonist/inhibitor of alkylglyerol monooxygenase (TMEM195; glyceryl ether monooxygenase; EC 1.14.16.5) comprising the steps of: (a) contacting a cell, tissue or a non-human animal comprising and expressing alkylglycerol monooxygenase with said candidate molecule; (b) detecting a decrease in alkylglycerol monooxygenase activity; and (c) selecting a candidate molecule that decreases alkylglycerol monooxygenase activity; wherein a decrease of the alkylglycerol monooxygenase activity is indicative for the capacity of the selected molecule to have an antiproliferative effect, to counteract hypertension, restore male fertility or to ameliorate or prevent cataract as already outlined above.

It is to be understood that the detected activity of alkylglycerol monooxygenase TMEM195 is compared to a standard or reference value of alkylglycerol monooxygenase TMEM195 activity. The standard/reference value may be detected in a cell, tissue, or non-human animal as defined herein, which has not been contacted with a potential alkylglycerol monooxygenase TMEM 195 inhibitor or prior to the above contacting step. The decrease in the activity of alkylglycerol monooxygenase TMEM195 may also be compared to the decrease in alkylglycerol monooxygenase TMEM 195 activity by (a) routinely used reference compound(s). A skilled person is easily in the position to determine/assess whether the activity and/or expression of alkylglycerol monooxygenase TMEM195 is (preferably statistically significant) decreased.

In accordance with this invention, in particular the screening or identifying methods described herein, a cell, tissue or non-human animal to be contacted with a candidate molecule comprises and expresses alkylglycerol monooxygenase TMEM195. For example said cell, tissue or non-human animal may express an alkylglycerol monooxygenase TMEM195 gene, in particular also (an) additional (copy) copies of a alkylglycerol monooxygenase TMEM195 gene, (a) alkylglycerol monooxygenase TMEM195 mutated gene(s), a recombinant alkylglycerol monooxygenase TMEM195 gene construct and the like. As explained herein below, the capability of a candidate molecule to inhibit/antagonize alkylglycerol monooxygenase TMEM 195 may, accordingly, be detected by measuring the expression level of such gene products of alkylglycerol monooxygenase TMEM195 or of corresponding gene constructs (e.g. mRNA or protein), wherein a low expression level (compared to a standard or reference value) is indicative for the capability of the candidate molecule to act as inhibitor/antagonist.

The term “candidate molecule” as used herein refers to a molecule or substance or compound or composition or agent or any combination thereof to be tested by one or more screening method(s) of the invention as a putative antagonist or inhibitor of alkylglycerol monooxygenase TMEM195 function, activity or expression. A test compound can be any chemical, such as an inorganic chemical, an organic chemical, a protein, a peptide, a carbohydrate, a lipid, or a combination thereof or any of the compounds, compositions or agents described herein. It is to be understood that the term “candidate molecule” when used in the context of the present invention is interchangeable with the terms “test compound”, “test molecule”, “test substance”, “potential candidate”, “candidate” or the terms mentioned herein above.

Also preferred potential candidate molecules or candidate mixtures of molecules to be used when contacting a cell expressing/comprising alkylglycerol monooxygenase TMEM195 as defined and described herein may be, inter alia, substances, compounds or compositions which are of chemical or biological origin, which are naturally occurring and/or which are synthetically, recombinantly and/or chemically produced. Thus, candidate molecules may be proteins, protein-fragments, peptides, amino acids and/or derivatives thereof or other compounds as defined herein, which bind to and/or interact with alkylglycerol monooxygenase TMEM195, regulatory proteins/sequences of alkylglycerol monooxygenase TMEM195 function or functional fragments thereof. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.) are readily producible. Additionally, natural and synthetically produced libraries and compounds are readily modified through conventional chemical, physical, and biochemical means. Results obtained from deorphanisation programs based on phylogenetic analysis methods may aid to find natural ligands for alkylglycerol monooxygenase TMEM195 and, e.g., will allow in silico profiling of potential ligands for alkylglycerol monooxygenase TMEM195.

The generation of chemical libraries with potential ligands for alkylglycerol monooxygenase TMEM195 is well known in the art. For example, combinatorial chemistry is used to generate a library of compounds to be screened in the assays described herein. A combinatorial chemical library is a collection of diverse chemical compounds generated by either chemical synthesis or biological synthesis by combining a number of chemical “building block” reagents. For example, a linear combinatorial chemical library such as a polypeptide library is formed by combining amino acids in every possible combination to yield peptides of a given length. Millions of chemical compounds can theoretically be synthesized through such combinatorial mixings of chemical building blocks. For example, one commentator observed that the systematic, combinatorial mixing of 100 interchangeable chemical building blocks results in the theoretical synthesis of 100 million tetrameric compounds or 10 billion pentameric compounds. (Gallop, Journal of Medicinal Chemistry, Vol. 37, No. 9, 1233-1250 (1994)). Other chemical libraries known to those in the art may also be used, including natural product libraries. Once generated, combinatorial libraries are screened for compounds that possess desirable biological properties. For example, compounds which may be useful as drugs or to develop drugs would likely have the ability to bind to the target protein identified, expressed and purified as described herein.

In the context of the present invention, libraries of compounds are screened to identify compounds that function as an antagonist or inhibitor of alkylglycerol monooxygenase TMEM195. First, a library of small molecules is generated using methods of combinatorial library formation well known in the art. U.S. Pat. No. 5,463,564 and U.S. Pat. No. 5,574,656 are two such teachings. Then the library compounds are screened to identify those compounds that possess desired structural and functional properties. U.S. Pat. No. 5,684,711, discusses a method for screening libraries. To illustrate the screening process, the target cell or gene product and chemical compounds of the library are combined and permitted to interact with one another. A labelled substrate is added to the incubation. The label on the substrate is such that a detectable signal is emitted from metabolized substrate molecules. The emission of this signal permits one to measure the effect of the combinatorial library compounds on the enzymatic activity of target enzymes/activity of target protein by comparing it to the signal emitted in the absence of combinatorial library compounds. The characteristics of each library compound are encoded so that compounds demonstrating activity against the cell/enzyme/target protein can be analyzed and features common to the various compounds identified can be isolated and combined into future iterations of libraries. Once a library of compounds is screened, subsequent libraries are generated using those chemical building blocks that possess the features shown in the first round of screen to have activity against the target protein. Using this method, subsequent iterations of candidate compounds will possess more and more of those structural and functional features required to inhibit the target protein, until a group of antagonists/inhibitors with high specificity for the protein can be found. These compounds can then be further tested for their safety and efficacy as an alkylglycerol monooxygenase TMEM195 inhibitor/antagonist agent for use in animals, such as mammals. It will be readily appreciated that this particular screening methodology is exemplary only. Other methods are well known to those skilled in the art. For example, a wide variety of screening techniques are known for a large number of naturally-occurring targets when the biochemical function of the target protein is known. For example, some techniques involve the generation and use of small peptides to probe and analyze target proteins both biochemically and genetically in order to identify and develop drug leads. Such techniques include the methods described in WO 99/35494, WO 98/19162, WO 99/54728.

Preferably, candidate agents to be tested encompass numerous chemical classes, though typically they are organic compounds, preferably small (organic) molecules as defined herein above.

Candidate agents may also comprise functional groups necessary for structural interaction with proteins, particularly hydrogen bonding, and typically include at least an amine, carbonyl, hydroxyl or carboxyl group, preferably at least two of the functional chemical groups. The candidate agents often comprise carbocyclic or heterocyclic structures and/or aromatic or polyaromatic structures substituted with one or more of the above functional groups.

Exemplary classes of candidate agents may include heterocycles, peptides, saccharides, steroids, and the like. The compounds may be modified to enhance efficacy, stability, pharmaceutical compatibility, and the like. Structural identification of an agent may be used to identify, generate, or screen additional agents. For example, where peptide agents are identified, they may be modified in a variety of ways to enhance their stability, such as using an unnatural amino acid, such as a D-amino acid, particularly D-alanine, by functionalizing the amino or carboxylic terminus, e.g. for the amino group, acylation or alkylation, and for the carboxyl group, esterification or amidification, or the like. Other methods of stabilization may include encapsulation, for example, in liposomes, etc.

As mentioned above, candidate agents are also found among other biomolecules including amino acids, fatty acids, purines, pyrimidines, nucleic acids and derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Step (a) of the screening method as defined herein above may be accomplished by contacting, e.g., the cell(s), tissue(s), or non-human-animal comprising and expressing alkylglycerol monooxygenase TMEM 195 with (a) candidate molecule(s) to be tested and it is measured whether said candidate molecule(s) lead(s) to a decrease in the activity of alkylglycerol monooxygenase TMEM 195. Such a change/decrease is indicative for the capacity of the candidate molecule to be useful in a condition wherein a decrease in the activity of a functional alkylglycerol monooxygenase is expected to have medical implications as outlined above, i.e., e.g., to have an antiproliferative effect, to counteract hypertension, restore male fertility or to ameliorate or prevent cateract. In other words, the activity of the candidate molecule(s) as inhibitors/antagonists of alkylglycerol monooxygenase TMEM195 is assessed based on their capacity to decrease the activity of alkylglycerol monooxygenase TMEM195 wherein a decrease of the alkylglycerol monooxygenase TMEM195 activity is indicative for the capacity of the selected molecule to ameliorate medical implications as outlined above, i.e., e.g., to have an antiproliferative effect, to counteract hypertension, to restore male fertility or to ameliorate or prevent cateract. In particular the use of (a) (transgenic) cell(s), tissue(s), or non-human animal(s) overexpressing alkylglycerol monooxygenase TMEM195 is envisaged, since these may allow a more sensitive/easier detection of a decrease of alkylglycerol monooxygenase TMEM195 activity.

It is to be understood that in a high throughput screening routinely, many (often thousands of candidate molecules) are screened simultaneously. Accordingly, in a (first) screen candidate molecules are selected, which decrease alkylglycerol monooxygenase TMEM195 activity.

Step (a) of the screening methods of the present invention, i.e. the “contacting step” may be accomplished by adding a candidate molecule or a (biological) sample or composition containing said candidate molecule or a plurality of candidate molecules (i.e. various different candidate molecules) to (a) cell(s)/tissue(s)/non-human animal comprising alkylglycerol monooxygenase TMEM 195 or a functional fragment thereof.

The term “contacting” may also refer to the addition of a candidate molecule a cell, tissue, non-human animal comprising alkylglycerol monooxygenase TMEM195 in a way that the candidate molecule may become effective to the cell at the cell surface or upon cellular uptake and thereby exert its inhibitory function on alkylglycerol monooxygenase TMEM195-dependent responses.

Generally, the candidate molecule(s) or a composition comprising/containing the candidate molecule(s) may for example be added to a cell, tissue or non-human animal comprising alkylglycerol monooxygenase TMEM195. As defined and disclosed herein, the term “comprising alkylglycerol monooxygenase TMEM195” refers not only to the alkylglycerol monooxygenase TMEM195 gene(s) or proteins known in the art and described herein, but also to reporter constructs comprising a reporter as described in detail below. Exemplary reporters (preferably associated with the reporter signals disclosed herein) are luciferase and fluorescent proteins, like GFP, RFP and the like. Also reporter constructs comprising a promoter and/or enhancer region of alkylglycerol monooxygenase TMEM195 can be used in the screening/identifying methods. Accordingly, the cell(s), tissue(s) and/or non-human animals used in the context of the present invention, in particular in context of the screening/identifying methods can be stably or transiently transfected with the reporter constructs disclosed herein.

In particular the identification/assessment of candidate molecules which are capable of inhibiting/antagonizing alkylglycerol monooxygenase TMEM195 may be, inter alia, performed by transfecting an appropriate host with a nucleic acid molecule encoding alkylglycerol monooxygenase TMEM195 (or a functional fragment thereof) and contacting said host with (a) candidate molecule(s). The host (cell, tissue, non-human animal) can also be transfected. The host cell may comprise, but is not limited to, CHO-cell, HEK 293, HeLa, Cos 7, PC12 or NIH3T3 cell, frog oocytes or primary cells like primary cardiomyocytes, fibroblasts, muscle, endothelial or embryonic stem cells. Alternatively, it is also possible to use cell lines stably transfected with a nucleic acid molecule encoding alkylglycerol monooxygenase TMEM 195 or a functional fragment thereof. The explanations given herein above in respect of “cells” also apply to tissues/non-human animals comprising or derived from these cells.

The (biological) sample or composition, comprising a plurality of candidate molecules are usually subject to a first screen. The samples/compositions tested positive in the first screen are often subject to subsequent screens in order to verify the previous findings and to select the most potent inhibitors/antagonists of alkylglycerol monooxygenase TMEM195. Upon multiple screening and selection rounds those candidate molecules will be selected which show a pronounced capacity to inhibit/antagonize alkylglycerol monooxygenase TMEM195 as defined and disclosed herein. For example, batches (i.e. compositions/samples) containing many candidate molecules will be rescreened and batches with no or insufficient inhibitory activity of candidate molecules be discarded without re-testing.

For example, if a (biological) sample or composition with many different candidate molecules is tested and one (biological) sample or composition is tested positive, then it is either possible in a second screening to screen, preferably after purification, the individual molecule(s) of the (biological) sample or composition. It may also be possible to screen subgroups of the (biological) sample or composition of the first screen in (a) subsequent screen(s). The screening of compositions with subgroups of those candidate molecules tested in previous screening rounds will thus narrow in on (an) potential potent alkylglycerol monooxygenase TMEM195 inhibitor(s). This may facilitate and accelerate the screening process in particular when a large number of molecules is screened. Accordingly, the cycle number of screening rounds is reduced compared to testing each and every individual candidate molecule in (a) first (and subsequent) screen(s) (which is, of course, also possible). Thus, depending on the complexity or the number of the candidate molecules, the steps of the screening method described herein can be performed several times until the (biological) sample or composition to be screened comprises a limited number, preferably only one substance which is indicative for the capacity of inhibiting alkylglycerol monooxygenase TMEM195 or decreasing the alkylglycerol monooxygenase TMEM195 activity wherein a decrease in the activity of a functional alkylglycerol monooxygenase is expected to have medical implications as outlined above, i.e., e.g., to have an antiproliferative effect, to counteract hypertension, restore male fertility or to ameliorate or prevent cateract.

The term “decrease in alkylglycerol monooxygenase TMEM195 activity” in step (b) of the screening method as described above means that the “activity of alkylglycerol monooxygenase TMEM195” is reduced upon contacting the cell, tissue, or non-human animal comprising alkylglycerol monooxygenase TMEM195 with the candidate molecule, preferably in comparison to a (control) standard or reference value, as defined herein wherein a decrease in the activity of a functional alkylglycerol monooxygenase is expected to have medical implications as outlined above, i.e., e.g., to have an antiproliferative effect, to counteract hypertension, restore male fertility or to ameliorate or prevent cateract.

As defined and disclosed herein, the term “comprising alkylglycerol monooxygenase TMEM195” refers not only to the alkylglycerol monooxygenase TMEM195 gene(s) or proteins known in the art and described herein. Also reporter constructs comprising a promoter and/or enhancer region of alkylglycerol monooxygenase TMEM195 can be used in the screening/identifying methods. Accordingly, the cell(s), tissue(s) and/or non-human animals used in the context of the present invention, in particular in context of the screening/identifying methods can comprise the reporter constructs disclosed herein and described below.

The activity of alkylglycerol monooxygenase TMEM195 can be quantified in cells, tissue or non-human animals.

The method for assessing the activity of a candidate molecule suspected of being an antagonist/inhibitor of alkylglycerol monooxygenase TMEM195 can be accomplished by determining a decrease in the activity of alkylglycerol monooxygenase TMEM195, wherein the decrease in alkylglycerol monooxygenase TMEM195 activity can be detected with polynucleotides capable of hybridizing the alkylglycerol monooxygenase TMEM195 sense molecule. Alternatively, the decrease in alkylglycerol monooxygenase TMEM 195 activity can be detected with antibodies capable of binding the alkylglycerol monooxygenase TMEM195 protein. Therefore, the activity of alkylglycerol monooxygenase TMEM195 can be quantified by measuring, for example, the level of gene products (e.g. mRNA and/or protein of alkylglycerol monooxygenase TMEM195) by any of the herein described methods, activities, the alkylglycerol monooxygenase TMEM195 concentration or other cellular functions. As mentioned herein above the candidate compound to be tested may lead to a modified activity of alkylglycerol monooxygenase TMEM195 and a decrease in the alkylglycerol monooxygenase TMEM195 activity is indicative for the capacity to antagonize alkylglycerol monooxygenase TMEM195 and thus to to have medical implications as outlined above, i.e., e.g., to have an antiproliferative effect, to counteract hypertension, to restore male fertility or to ameliorate or prevent cateract.

As mentioned, a “decreased alkylglycerol monooxygenase TMEM195 activity” and, accordingly, a decreased concentration/amount of alkylglycerol monooxygenase TMEM195 proteins in a sample may be reflected in a decreased expression of the corresponding gene(s) encoding the alkylglycerol monooxygenase TMEM195 protein(s). Therefore, a quantitative assessment of the gene product (e.g. protein or spliced, unspliced or partially spliced mRNA) can be performed in order to evaluate decreased expression of the corresponding gene(s) encoding the alkylglycerol monooxygenase TMEM195 protein(s). Also here, a person skilled in the art is aware of standard methods to be used in this context or may deduce these methods from standard textbooks (e.g. Sambrook, 2001, loc. cit.). For example, quantitative data on the respective concentration/amounts of mRNA from alkylglycerol monooxygenase TMEM195 can be obtained by Northern Blot, Real Time PCR and the like. Preferably, the concentration/amount of the gene product (e.g. the herein above described alkylglycerol monooxygenase TMEM195 mRNA or alkylglycerol monooxygenase TMEM195 protein) may be decreased by at least about 10%, 20%, 30%, 40%, preferably by at least 50%, 60%, 70%, 80%, 90%, or 100% compared to a control sample. It is preferred herein that alkylglycerol monooxygenase TMEM195 proteins are (biologically) active or functional. Methods for determining the activity of alkylglycerol monooxygenase TMEM195 are described herein above and below. Since the alkylglycerol monooxygenase TMEM195 proteins are preferably (biologically) active/functional (wherein it is preferred that at least 70%, 75%, preferably at least 80%, 85%, 90%, 95%, 96, %, 97%, 98% and most preferably, at least 99% of alkylglycerol monooxygenase TMEM195 proteins of a sample a (biologically) active/functional), an decreased concentration/amount of alkylglycerol monooxygenase TMEM195 proteins in a sample reflects a decreased (biological) activity of the alkylglycerol monooxygenase TMEM195 protein.

As mentioned, a person skilled in the art is aware of standard methods to be used for determining or quantitating expression of a nucleic acid molecule encoding, for example, the alkylglycerol monooxygenase TMEM195 (or fragments thereof). For example, the expression can be determined on the protein level by taking advantage of immunoagglutination, immunoprecipitation (e.g. immunodiffusion, immunelectrophoresis, immune fixation), western blotting techniques (e.g. (in situ) immuno histochemistry, (in situ) immuno cytochemistry, affinitychromatography, enzyme immunoassays), and the like. Amounts of purified polypeptide in solution can be determined by physical methods, e.g. photometry.

Methods of quantifying a particular polypeptide in a mixture rely on specific binding, e.g of antibodies. Specific detection and quantitation methods exploiting the specificity of antibodies comprise for example immunohistochemistry (in situ). For example, concentration/amount of alkylglycerol monooxygenase TMEM195 proteins in a cell, tissue or a non-human animal can be determined by enzyme linked-immunosorbent assay (ELISA). Alternatively, Western Blot analysis or immunohistochemical staining can be performed. Western blotting combines separation of a mixture of proteins by electrophoresis and specific detection with antibodies. Electrophoresis may be multi-dimensional such as 2D electrophoresis. Usually, polypeptides are separated in 2D electrophoresis by their apparent molecular weight along one dimension and by their isoelectric point along the other direction.

Expression can also be determined on the nucleic acid level (e.g. if the gene product/product of the coding nucleic acid sequence is an unspliced/partially spliced/spliced mRNA) by taking advantage of Northern blotting techniques or PCR techniques, like in-situ PCR or Real time PCR. Quantitative determination of mRNA can be performed by taking advantage of northern blotting techniques, hybridization on microarrays or DNA chips equipped with one or more probes or probe sets specific for mRNA transcripts or PCR techniques referred to above, like, for example, quantitative PCR techniques, such as Real time PCR.

These and other suitable methods for detection and/or determination of the concentration/amount of (specific) mRNA or protein(s)/polypeptide(s) are well known in the art and are, for example, described in Sambrook (2001), loc. cit.).

A skilled person is capable of determining the amount of mRNA or polypeptides/proteins, in particular the gene products described herein above, by taking advantage of a correlation, preferably a linear correlation, between the intensity of a detection signal and the amount of, for example, the mRNA or polypeptides/proteins to be determined.

The difference, as disclosed herein is statistically significant and a candidate molecule(s) is (are) selected, if the alkylglycerol monooxygenase TMEM195 activity (or of a corresponding reporter signal) is strongly decreased, preferably is very low or non-detectable. For example, the alkylglycerol monooxygenase TMEM195 activity (or of a corresponding reporter signal) may be decreased by at least 50%, 60%, 70%, 80%, more preferably by at least 90% compared to the (control) standard value. In a cell based method the cells can be transfected with one or more constructs encoding alkylglycerol monooxygenase TMEM195 or a functional fragment thereof.

Preferably, the selected compound has a high alkylglycerol monooxygenase TMEM195 inhibiting/antagonizing activity. This can be reflected in the capacity of the alkylglycerol monooxygenase TMEM195 antagonist/inhibitor to potently decrease the activity of alkylglycerol monooxygenase TMEM195.

The above detected difference between the activity of alkylglycerol monooxygenase TMEM195 or the activity of a functional fragment of alkylglycerol monooxygenase TMEM195 in a cell, tissue or a non-human animal contacted with said candidate molecule and the activity in the (control) standard value (measured e.g. in the absence of said candidate molecule) may be reflected by the presence, the absence, the increase or the decrease of a specific signal in the readout system.

Genetic readout systems are also envisaged. Analogously, the activity of alkylglycerol monooxygenase TMEM195 or of a functional fragment thereof may be quantified by any molecular biological method as described herein. A skilled person is also aware of standard methods to be used in determining the amount/concentration of alkylglycerol monooxygenase TMEM195 expression products (in particular the protein and the nucleic acid level of alkylglycerol monooxygenase TMEM 195) in a sample or may deduce corresponding methods from standard textbooks (e.g. Sambrook, 2001).

Also in this context, reporter constructs comprising a promoter and/or enhancer region of alkylglycerol monooxygenase TMEM195 can be used in the screening/identifying methods. Exemplary reporters (preferably associated with the reporter signals disclosed herein) are luciferase and fluorescent proteins, like GFP, RFP and the like. It is preferred that a promoter and/or enhancer element/region of alkylglycerol monooxygenase TMEM195 is used in this context and is fused to a reporter.

Exemplary reporter signals, reporters and reporter constructs are described herein below. Interesting reporters, namely reporter gene products, which can be used in the screening and identifying methods of the invention like luciferase, (green/red) fluorescent protein and variants thereof, EGFP (enhanced green fluorescent protein), RFP (red fluorescent protein, like DsRed or DsRed2), CFP (cyan fluorescent protein), BFP (blue green fluorescent protein), YFP (yellow fluorescent protein), β-galactosidase or chloramphenicol acetyltransferase as well as methods for their detection are also described herein below in detail. Luciferase is a well known reporter; see, for example, Jeffrey (1987) Mol. Cell. Biol. 7(2), 725-737. A person skilled in the art can easily deduce further luciferase nucleic and amino acid sequences to be used in context of the present invention from corresponding databases and standard text books/review.

Further exemplary reporter constructs to be employed in context of the present invention, in particular the screening and identifying methods, comprise promoter(s) (and/or (a) enhancer region(s)) of alkylglycerol monooxygenase TMEM195, wherein the (initiation/enhancement of the) expression of the reporter(s) is under control of the promoter and/or enhancer of alkylglycerol monooxygenase TMEM195. A skilled person may easily retrieve these and other well-known sequences from databases (like NCBI) and use these sequences in the generation of reporter constructs to be employed herein. For example, the alkylglycerol monooxygenase TMEM 195 promoter sequence the corresponding database.

These reporter constructs comprising a reporter and a promoter (and/or enhancer) as defined above, are particularly useful in screening methods and assays, since the reporter signal associated with the reporter can easily be detected. A change in the reporter signal is indicative for the capacity of a candidate molecule tested to act as antagonist/inhibitor of alkylglycerol monooxygenase TMEM195. For example, an antagonist of alkylglycerol monooxygenase TMEM195 will lead to a decrease of a reporter signal/activity of a reporter under control of the alkylglycerol monooxygenase TMEM 195 promoter. In particular, a reporter construct may comprise a luciferase gene and a promoter of alkylglycerol monooxygenase TMEM195. A person skilled in the art is easily in the position to generate this and other reporter constructs using routine techniques. Inter alia, vectors such as the pRL-TK RENILLA Vector and other well known vectors may be employed in the generation of the reporter constructs.

Apparently, decreased expression of the reporter gene/activity of the reporter gene product will reflect a decreased alkylglycerol monooxygenase TMEM195 activity, in particular a decreased concentration/amount of alkylglycerol monooxygenase TMEM195 protein. Alternatively, the effect of the antagonist/inhibitor on the expression of (a) reporter gene(s) may be evaluated by determining the amount/concentration of the gene product of the reporter gene(s) (e.g. protein or spliced, unspliced or partially spliced mRNA). Further methods to be used in the assessment of mRNA expression of a reporter gene are within the scope of a skilled person and also described herein below.

As mentioned above, it is preferred that a promoter and/or enhancer element/region of alkylglycerol monooxygenase TMEM195 is used in this context and is fused to a reporter. As used herein, the term “reporter construct for alkylglycerol monooxygenase TMEM195-inhibition” relates to any biotechnologically engineered construct allowing the detection of alkylglycerol monooxygenase TMEM195 inhibition. Accordingly, said reporter construct may allow the detection of alkylglycerol monooxygenase TMEM195-inhibition by inducing a change in the signal strength of a detectable signal. Said detectable signal may be selected from the group consisting of, but not limited to a fluorescence resonance energy transfer (FRET) signal, a fluorescence polarization (FP) signal and a scintillation proximity (SP) signal. In a further embodiment, said detectable signal may be associated with a reporter gene product. Examples of reporter gene products include luciferase, (green/red) fluorescent protein and variants thereof, like EGFP (enhanced green fluorescent protein), RFP (red fluorescent protein, like DsRed or DsRed2), CFP (cyan fluorescent protein), BFP (blue green fluorescent protein), YFP (yellow fluorescent protein), β-galactosidase or chloramphenicol acetyltransferase, and the like. For example, GFP can be derived from Aequorea victoria (U.S. Pat. No. 5,491,084). A plasmid encoding the GFP of Aequorea victoria is available from the ATCC Accession No. 87451. Other mutated forms of this GFP including, but not limited to, pRSGFP, EGFP, RFP/DsRed, DSRed2, and EYFP, BFP, YFP, among others, are commercially available from, inter alia, Clontech Laboratories, Inc. (Palo Alto, Calif.).

In another preferred embodiment, the non-human animal comprising said reporter construct for detecting alkylglycerol monooxygenase TMEM195 inhibition is a transgenic non-human animal. The non-human organism to be used in the described screening assays is preferably selected from the group consisting of C. elegans, yeast, drosophila, zebrafish, guinea pig, rat and mouse. The generation of such a transgenic animal is within the skill of a skilled artisan. Corresponding techniques are, inter alia, described in “Current Protocols in Neuroscience” (2001), John Wiley&Sons, Chapter 3.16. Accordingly, the invention also relates to a method for the generation of a non-human transgenic animal comprising the step of introducing a reporter construct for detecting alkylglycerol monooxygenase TMEM 195 inhibition as disclosed herein into an ES-cell or a germ cell. The non-human transgenic animal provided and described herein is particular useful in screening methods and pharmacological tests described herein above. In particular the non-human transgenic animal described herein may be employed in drug screening assays as well as in scientific and medical studies wherein antagonists/inhibitors of alkylglycerol monooxygenase TMEM195 for the treatment of a disease related to to have an antiproliferative effect, to counteract hypertension, to restore male fertility or to ameliorate or prevent cateract tracked, selected and/or isolated.

The transgenic/genetically engineered cell(s), tissue(s), and/or non-human animals to be used in context of the present invention, in particular, the screening/identifying methods, preferably comprise the herein described and defined reporter constructs. Hence, in this context, reporter constructs may comprise a promoter and/or enhancer region of alkylglycerol monooxygenase TMEM195 as defined herein. Exemplary reporters (preferably associated with the reporter signals disclosed herein) are luciferase and fluorescent proteins, like GFP, RFP and the like. Exemplary, non-limiting constructs to be used may comprise a luciferase reporter under control of a (human) alkylglycerol monooxygenase TMEM195 promoter and/or enhancer region. Exemplary reporters are luciferase and fluorescent proteins, like GFP, RFP and the like.

The method for assessing the activity of a candidate molecule suspected of being an antagonist/inhibitor of alkylglycerol monooxygenase TMEM195 can be accomplished by determining a decrease in the activity of alkylglycerol monooxygenase TMEM 195, wherein the decrease in alkylglycerol monooxygenase TMEM 195 activity can be detected with polynucleotides capable of hybridizing the alkylglycerol monooxygenase TMEM195 sense molecule.

In a preferred embodiment, the method for assessing the activity of a candidate molecule suspected of being an antagonist/inhibitor of alkylglycerol monooxygenase TMEM195 can be accomplished by determining a decrease in the activity of alkylglycerol monooxygenase TMEM195, wherein the decrease in alkylglycerol monooxygenase TMEM195 activity can be detected by monitoring the enzyme activity of the alkylglycerol monooxygenase TMEM195 itself, wherein an decrease of the alkylglycerol monooxygenase TMEM195 activity is indicative for the capacity of the selected molecule to to have medical implications as outlined above, i.e., e.g., to have an antiproliferative effect, to counteract hypertension or to ameliorate or prevent cateract. A cell to be used is a cell that comprises and expresses alkylglycerol monooxygenase TMEM195. The alkylglycerol monooxygenase TMEM195 activity can be quantified in cells, tissue or non-human animals can be assayed as described in the appended examples. Hence, alkylglycerol monooxygenase TMEM195 can, e.g., be performed as described in the following: A pyrene-labelled alkylglycerol (1-O-pyrenedecyl-sn-glycerol) was used as substrate which was converted to pyrenedecanal by alkylglycerol monooxygenase. Since pyrenedecanal is stable to aerobic oxidation and cannot not be sufficiently separated from the 1-O-pyreneglycerol substrate by our HPLC system, the assay requires the presence of fatty aldehyde dehydrogenase in the sample which converts pyrenedecanal to pyrenedecanoic acid, the product finally detected by HPLC. Fatty aldehyde dehydrogenase is abundant in mouse tissues, and is also present in CHO cells (18.8±1.3 pmol mg⁻¹ min⁻¹, mean±SEM, n=4). In some recombinant expression experiments, fatty aldehyde dehydrogenase in CHO cells was increased by recombinant overexpression, or supplied to Xenopus laevis oocytes by cRNA injection. For protein purification experiments, 14 pmol ml⁻¹ min⁻¹ recombinant rat fatty aldehyde dehydrogenase was added to the reaction mixture. 10 μl alkylglycerol monooxygenase reaction mixture contained 100 mM Tris HCl pH 8.5, 0.1 mg/ml catalase, 0.2 mM NAD, 0.2 mM NADPH (all from Sigma), 0.1 mM 1-O-pyrenedecyl-sn-glycerol (chemically synthesized from pyrenedecanoic acid and glycerol as described (2)), 0.2 μg/ml (0.5 μmol ml⁻¹ min⁻¹) recombinant Physarum polycephalum dihydropteridine reductase (4) and 0.2 mM tetrahydrobiopterin (Schircks, Jona, Switzerland). The reaction was started by addition of the protein and incubated for 60 min at 37° C. in the dark. Negative controls without protein (concentration of pyrenedecanoic acid <1 nM) and rat liver microsomes as positive controls were always run in parallel. After addition of 30 μl methanol and centrifugation for 5 min at 16,000 g, 10 μl of the sample were injected to a Zorbax XDB-C8 rapid resolution column (Agilent Technologies, Vienna, Austria) using an Agilent 1200 Series HPLC system. Elution (flow rate 1.0 ml/min) was performed with a mixture of 21% (v/v) 10 mM potassium phosphate buffer, pH 6.0 and 79% (v/v) methanol for 4.5 min, followed by a gradient to 100% methanol at 5.0 min. At 8.0 min, the initial buffer/methanol (21:79) mix was re-established and the column equilibrated until 8.5 min. Pyrenedecanoic acid was detected by fluorescence (340 nm excitation and 400 nm emission, detection limit 1 nM).

In a preferred embodiment, the method for assessing the activity of a candidate molecule suspected of being an antagonist/inhibitor of alkylglycerol monooxygenase TMEM195 can be accomplished by determining the physical interaction/binding of candidate molecules with alkylglycerol monooxygenase TMEM195. Interaction methods are known in the art. Interaction assays employing read-out systems are well known in the art and comprise, inter alia, two hybrid screenings (as, described, inter alia, in EP-0 963 376, WO 98/25947, WO 00/02911), GST-pull-down columns, co-precipitation assays from cell extracts as described, inter alia, in Kasus-Jacobi (2000) Oncogene 19:2052-2059, “interaction-trap” systems (as described, inter alia, in U.S. Pat. No. 6,004,746) expression cloning (e.g. lamda gtII), phage display (as described, inter alia, in U.S. Pat. No. 5,541,109), in vitro binding assays and the like. Further interaction assay methods and corresponding read out systems are, inter alia, described in U.S. Pat. No. 5,525,490, WO 99/51741, WO 00/17221, WO 00/14271, WO 00/05410 or Yeast Four hybrid assays as described in Sandrock (2001) J. Biol. Chem. 276:35328-35333.

Said interaction assays for alkylglycerol monooxygenase TMEM195 also comprise assays for FRET-assays, TR-FRETs (in “A homogenius time resolved fluorescence method for drug discovery” in: High throughput screening: the discovery of bioactive substances. Kolb (1997) J. Devlin. NY, Marcel Dekker 345-360) or commercially available assays, like “Amplified Luminescent Proximity Homogenous Assay”, BioSignal Packard. Furthermore, the yeast-2-hybrid (Y2H) system may be employed to elucidate further particular and specific interaction, association partners of alkylglycerol monooxygenase TMEM195. Said interaction/association partners are suspected of being an antagonist/inhibitor of alkylglycerol monooxygenase TMEM195 and are further screened for their antagonistic/inhibiting effects as described above. As mentioned herein above, the candidate compound that interacts with alkylglycerol monooxygenase TMEM195 may lead to a modified activity of alkylglycerol monooxygenase TMEM195 and a decrease in the alkylglycerol monooxygenase TMEM195 activity is indicative for the capacity to antagonize alkylglycerol monooxygenase TMEM 195 and thus to have medical implications as outlined above, i.e., e.g., to have an antiproliferative effect, to counteract hypertension, to restore male infertility or to ameliorate or prevent cateract.

Similarly, interacting molecules (for example) (poly)peptides may be deduced by cell-based techniques well known in the art. These assays comprise, inter alia, the expression of reporter gene constructs or “knock-in” assays, as described, for, e.g., the identification of drugs/small compounds influencing the (gene) expression of alkylglycerol monooxygenase TMEM195. Said “knock-in” assays may comprise “knock-in” of alkylglycerol monooxygenase TMEM195 (or (a) fragment(s) thereof) in tissue culture cells, as well as in (transgenic) animals. Examples for successful “knock-ins” are known in the art (see, inter alia, Tanaka (1999) Neurobiol. 41:524-539 or Monroe (1999) Immunity 11:201-212). Furthermore, biochemical assays may be employed which comprise, but are not limited to, binding of the alkylglycerol monooxygenase TMEM195 (or (a) fragment(s) thereof) to other molecules/(poly)peptides, peptides or binding of the alkylglycerol monooxygenase TMEM195 (or (a) fragment(s) thereof) to itself (themselves) (dimerizations, oligomerizations, multimerizations) and assaying said interactions by, inter alia, scintillation proximity assay (SPA) or homogenous time-resolved fluorescence assay (HTRFA).

Said “testing of interaction” may also comprise the measurement of a complex formation. The measurement of a complex formation is well known in the art and comprises, inter alia, heterogeneous and homogeneous assays. Homogeneous assays comprise assays wherein the binding partners remain in solution and comprise assays, like agglutination assays. Heterogeneous assays comprise assays like, inter alia, immuno assays, for example, ELISAs, RIAs, IRMAs, FIAs, CLIAs or ECLs.

The interaction of the antagonistic molecules of alkylglycerol monooxygenase TMEM195 mRNA and alkylglycerol monooxygenase TMEM195 protein or fragments thereof may also be tested by molecular biological methods, like two-, three- or four-hybrid-assays, RNA protection assays, Northern blots, Western blots, micro-, macro- and protein- or antibody arrays, dot blot assays, in situ hybridization and immunohistochemistry, quantitative PCR, coprecipitation, far western blotting, phage based expression cloning, surface plasmon resonance measurements, yeast one hybrid screening, DNAse I, footprint analysis, mobility shift DNA-binding assays, gel filtration chromatography, affinity chromatography, immunoprecipitation, one- or two dimensional gel electrophoresis, aptamer technologies, as well as high throughput synthesis and screening methods.

In sum, the present invention provides for the first time methods for identifying, and characterizing (a) candidate molecule(s) or (a) compound(s) which are capable of inhibiting/antagonizing alkylglycerol monooxygenase TMEM195 whereby said inhibition may lead to an decrease in the activity of a functional alkylglycerol monooxygenase is expected to have medical implications as outlined above, i.e., e.g., to have an antiproliferative effect, to counteract hypertension or to ameliorate or prevent cateract. Therefore the present invention provides for screening as well as identification methods for antagonists of alkylglycerol monooxygenase TMEM195. As also disclosed herein above, the term “antagonist” relates to molecules or compounds that bind to alkylglycerol monooxygenase TMEM195 or a functional fragment thereof, thereby inhibiting and/or reducing alkylglycerol monooxygenase TMEM195 activity, wherein these alkylglycerol monooxygenase TMEM195 antagonists have an antiproliferative effect, to counteract hypertension, to restore male fertility or to ameliorate or prevent cateract.

In a further embodiment, the present invention also provides for the first time a method for assessing the activity of a candidate molecule suspected of being an agonist/activator of alkylglyerol monooxygenase (TMEM195; glyceryl ether monooxygenase; EC 1.14.16.5) comprising the steps of: (a) contacting a cell, tissue or a non-human animal comprising and expressing alkylglycerol monooxygenase with said candidate molecule; (b) detecting a decrease in alkylglycerol monooxygenase activity; and (c) selecting a candidate molecule that decreases alkylglycerol monooxygenase activity; wherein an increase of the alkylglycerol monooxygenase activity is indicative for the capacity of the selected molecule to ameliorate both the neurodegeneration or to ameliorate the recent memory loss associated with Alzheimer's disease and to elicit/induce male infertility.

The compounds capable of increasing alkylglycerol monooxygenase TMEM195 function or (a) fragment(s) thereof, are expected to be very beneficial as agents in pharmaceutical settings disclosed herein and to be used for medical purposes, in particular, in the elicitation of male infertility, in the treatment of neurodegeneration or recent memory loss associated with Alzheimer's disease as already described in detail above.

Accordingly, screening methods for agonists/activators of alkylglycerol monooxygenase TMEM195 in cells, tissue and/or a non-human animal are provided. Also identification methods for agonists of alkylglycerol monooxygenase TMEM195 are provided. These methods are highly useful in identifying/screening (a) candidate molecule(s) suspected of being activators of alkylglycerol monooxygenase TMEM195 activity. Potent activators identified/screened by these methods can be used in the medical intervention of a condition wherein an increase in the activity of a functional alkylglycerol monooxygenase is expected to have medical implications as outlined above i.e., e.g., in the treatment of male infertility, neurodegeneration or recent memory loss associated with Alzheimer's disease as already described in detail above. In accordance with the present invention, a candidate molecule that may be suspected of being an agonist of alkylglycerol monooxygenase TMEM195 can, in principle, be obtained from any source as defined herein. The candidate molecule(s) may be (a) naturally occurring substance(s) or (a) substance(s) produced by a transgenic organism and optionally purified to a certain degree and/or further modified as described herein. Practically, the candidate molecule may be taken from a compound library as they are routinely applied for screening processes.

It is to be understood that the detected activity of alkylglycerol monooxygenase TMEM195 is compared to a standard or reference value of alkylglycerol monooxygenase TMEM195 activity. The standard/reference value may be detected in a cell, tissue, or non-human animal as defined herein, which has not been contacted with a potential alkylglycerol monooxygenase TMEM 195 activator or prior to the above contacting step. The increase in the activity of alkylglycerol monooxygenase TMEM195 may also be compared to the increase in alkylglycerol monooxygenase TMEM 195 activity by (a) routinely used reference compound(s). A skilled person is easily in the position to determine/assess whether the activity and/or expression of alkylglycerol monooxygenase TMEM 195 is (preferably statistically significant) increased.

In accordance with this invention, in particular the screening or identifying methods described herein, a cell, tissue or non-human animal to be contacted with a candidate molecule comprises and expresses alkylglycerol monooxygenase TMEM195. For example said cell, tissue or non-human animal may express a alkylglycerol monooxygenase TMEM195 gene, in particular also (an) additional (copy) copies of a alkylglycerol monooxygenase TMEM195 gene, (a) alkylglycerol monooxygenase TMEM195 mutated gene(s), a recombinant alkylglycerol monooxygenase TMEM195 gene construct and the like. As explained herein below, the capability of a candidate molecule to activate alkylglycerol monooxygenase TMEM195 may, accordingly, be detected by measuring the expression level of such gene products of alkylglycerol monooxygenase TMEM195 or of corresponding gene constructs (e.g. mRNA or protein), wherein a high expression level (compared to a standard or reference value) is indicative for the capability of the candidate molecule to act as agonist/activator.

The term “candidate molecule” as used herein refers to a molecule or substance or compound or composition or agent or any combination thereof to be tested by one or more screening method(s) of the invention as a putative agonist or activator of alkylglycerol monooxygenase TMEM195 function, activity or expression. A test compound can be any chemical, such as an inorganic chemical, an organic chemical, a protein, a peptide, a carbohydrate, a lipid, or a combination thereof or any of the compounds, compositions or agents described herein. It is to be understood that the term “candidate molecule” when used in the context of the present invention is interchangeable with the terms “test compound”, “test molecule”, “test substance”, “potential candidate”, “candidate” or the terms mentioned herein above.

Also preferred potential candidate molecules or candidate mixtures of molecules to be used when contacting a cell expressing/comprising alkylglycerol monooxygenase TMEM195 as defined and described herein may be, inter alia, substances, compounds or compositions which are of chemical or biological origin, which are naturally occurring and/or which are synthetically, recombinantly and/or chemically produced. Thus, candidate molecules may be proteins, protein-fragments, peptides, amino acids and/or derivatives thereof or other compounds as defined herein, which bind to and/or interact with alkylglycerol monooxygenase TMEM 195, regulatory proteins/sequences of alkylglycerol monooxygenase TMEM 195 function or functional fragments thereof. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.) are readily producible. Additionally, natural and synthetically produced libraries and compounds are readily modified through conventional chemical, physical, and biochemical means. Results obtained from deorphanisation programs based on phylogenetic analysis methods may aid to find natural ligands for alkylglycerol monooxygenase TMEM195 and, e.g., will allow in silico profiling of potential ligands for alkylglycerol monooxygenase TMEM195.

The generation of chemical libraries with potential ligands for alkylglycerol monooxygenase TMEM195 is well known in the art. For example, combinatorial chemistry is used to generate a library of compounds to be screened in the assays described herein. A combinatorial chemical library is a collection of diverse chemical compounds generated by either chemical synthesis or biological synthesis by combining a number of chemical “building block” reagents. For example, a linear combinatorial chemical library such as a polypeptide library is formed by combining amino acids in every possible combination to yield peptides of a given length. Millions of chemical compounds can theoretically be synthesized through such combinatorial mixings of chemical building blocks. For example, one commentator observed that the systematic, combinatorial mixing of 100 interchangeable chemical building blocks results in the theoretical synthesis of 100 million tetrameric compounds or 10 billion pentameric compounds. (Gallop, Journal of Medicinal Chemistry, Vol. 37, No. 9, 1233-1250 (1994)). Other chemical libraries known to those in the art may also be used, including natural product libraries. Once generated, combinatorial libraries are screened for compounds that possess desirable biological properties. For example, compounds which may be useful as drugs or to develop drugs would likely have the ability to bind to the target protein identified, expressed and purified as described herein.

In the context of the present invention, libraries of compounds are screened to identify compounds that function as an agonist or activator of alkylglycerol monooxygenase TMEM195. First, a library of small molecules is generated using methods of combinatorial library formation well known in the art. U.S. Pat. No. 5,463,564 and U.S. Pat. No. 5,574,656 are two such teachings. Then the library compounds are screened to identify those compounds that possess desired structural and functional properties. U.S. Pat. No. 5,684,711, discusses a method for screening libraries. To illustrate the screening process, the target cell or gene product and chemical compounds of the library are combined and permitted to interact with one another. A labelled substrate is added to the incubation. The label on the substrate is such that a detectable signal is emitted from metabolized substrate molecules. The emission of this signal permits one to measure the effect of the combinatorial library compounds on the enzymatic activity of target enzymes/activity of target protein by comparing it to the signal emitted in the absence of combinatorial library compounds. The characteristics of each library compound are encoded so that compounds demonstrating activity against the cell/enzyme/target protein can be analyzed and features common to the various compounds identified can be isolated and combined into future iterations of libraries. Once a library of compounds is screened, subsequent libraries are generated using those chemical building blocks that possess the features shown in the first round of screen to have activity against the target protein. Using this method, subsequent iterations of candidate compounds will possess more and more of those structural and functional features required to inhibit the target protein, until a group of agonists/activators with high specificity for the protein can be found. These compounds can then be further tested for their safety and efficacy as an alkylglycerol monooxygenase TMEM195 agonist/activator agent for use in animals, such as mammals. It will be readily appreciated that this particular screening methodology is exemplary only. Other methods are well known to those skilled in the art. For example, a wide variety of screening techniques are known for a large number of naturally-occurring targets when the biochemical function of the target protein is known. For example, some techniques involve the generation and use of small peptides to probe and analyze target proteins both biochemically and genetically in order to identify and develop drug leads. Such techniques include the methods described in WO 99/35494, WO 98/19162, WO 99/54728.

Preferably, candidate agents to be tested encompass numerous chemical classes, though typically they are organic compounds, preferably small (organic) molecules as defined herein above.

Candidate agents may also comprise functional groups necessary for structural interaction with proteins, particularly hydrogen bonding, and typically include at least an amine, carbonyl, hydroxyl or carboxyl group, preferably at least two of the functional chemical groups. The candidate agents often comprise carbocyclic or heterocyclic structures and/or aromatic or polyaromatic structures substituted with one or more of the above functional groups.

Exemplary classes of candidate agents may include heterocycles, peptides, saccharides, steroids, and the like. The compounds may be modified to enhance efficacy, stability, pharmaceutical compatibility, and the like. Structural identification of an agent may be used to identify, generate, or screen additional agents. For example, where peptide agents are identified, they may be modified in a variety of ways to enhance their stability, such as using an unnatural amino acid, such as a D-amino acid, particularly D-alanine, by functionalizing the amino or carboxylic terminus, e.g. for the amino group, acylation or alkylation, and for the carboxyl group, esterification or amidification, or the like. Other methods of stabilization may include encapsulation, for example, in liposomes, etc.

As mentioned above, candidate agents are also found among other biomolecules including amino acids, fatty acids, purines, pyrimidines, nucleic acids and derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Step (a) of the screening method as defined herein above may be accomplished by contacting, e.g., the cell(s), tissue(s), or non-human-animal comprising and expressing alkylglycerol monooxygenase TMEM 195 with (a) candidate molecule(s) to be tested and it is measured whether said candidate molecule(s) lead(s) to a increase in the activity of alkylglycerol monooxygenase TMEM195. Such a change/increase is indicative for the capacity of the candidate molecule to be useful in a condition wherein an increase in the activity of a functional alkylglycerol monooxygenase is expected to have medical implications as outlined above, i.e., e.g., in the treatment of male infertility, neurodegeneration or recent memory loss associated with Alzheimer's disease as already described in detail above. In other words, the activity of the candidate molecule(s) as agonist/activator of alkylglycerol monooxygenase TMEM195 is assessed based on their capacity to increase the activity of alkylglycerol monooxygenase TMEM195 wherein an increase of the alkylglycerol monooxygenase TMEM195 activity is indicative for the capacity of the selected molecule to ameliorate medical implications as outlined above, i.e., e.g., to counteract neurodegeneration or recent memory loss associated with Alzheimer's disease, or to elicit male infertility as already described in detail above. In particular the use of (a) (transgenic) cell(s), tissue(s), or non-human animal(s) with a reduced alkylglycerol monooxygenase TMEM195 expression is envisaged, since these may allow a more sensitive/easier detection of an increase of alkylglycerol monooxygenase TMEM195 activity.

It is to be understood that in a high throughput screening routinely, many (often thousands of candidate molecules) are screened simultaneously. Accordingly, in a (first) screen candidate molecules are selected, which increase alkylglycerol monooxygenase TMEM195 activity.

Step (a) of the screening methods of the present invention, i.e. the “contacting step” may be accomplished by adding a candidate molecule or a (biological) sample or composition containing said candidate molecule or a plurality of candidate molecules (i.e. various different candidate molecules) to (a) cell(s)/tissue(s)/non-human animal comprising alkylglycerol monooxygenase TMEM195 or a functional fragment thereof.

The term “contacting” may also refer to the addition of a candidate molecule a cell, tissue, non-human animal comprising alkylglycerol monooxygenase TMEM195 in a way that the candidate molecule may become effective to the cell at the cell surface or upon cellular uptake and thereby exert its inhibitory function on alkylglycerol monooxygenase TMEM195-dependent responses.

Generally, the candidate molecule(s) or a composition comprising/containing the candidate molecule(s) may for example be added to a cell, tissue or non-human animal comprising alkylglycerol monooxygenase TMEM195. As defined and disclosed herein, the term “comprising alkylglycerol monooxygenase TMEM195” refers not only to the alkylglycerol monooxygenase TMEM 195 gene(s) or proteins known in the art and described herein, but also to reporter constructs comprising a reporter as described in detail below. Exemplary reporters (preferably associated with the reporter signals disclosed herein) are luciferase and fluorescent proteins, like GFP, RFP and the like. Also reporter constructs comprising a promoter and/or enhancer region of alkylglycerol monooxygenase TMEM195 can be used in the screening/identifying methods. Accordingly, the cell(s), tissue(s) and/or non-human animals used in the context of the present invention, in particular in context of the screening/identifying methods can be stably or transiently transfected with the reporter constructs disclosed herein.

In particular the identification/assessment of candidate molecules which are capable of activating alkylglycerol monooxygenase TMEM195 may be, inter alia, performed by transfecting an appropriate host with a nucleic acid molecule encoding alkylglycerol monooxygenase TMEM195 (or a functional fragment thereof) and contacting said host with (a) candidate molecule(s). The host (cell, tissue, non-human animal) can also be transfected. The host cell may comprise, but is not limited to, CHO-cell, HEK 293, HeLa, Cos 7, PC12 or NIH3T3 cell, frog oocytes or primary cells like primary cardiomyocytes, fibroblasts, muscle, endothelial or embryonic stem cells. Alternatively, it is also possible to use cell lines stably transfected with a nucleic acid molecule encoding alkylglycerol monooxygenase TMEM195 or a functional fragment thereof. The explanations given herein above in respect of “cells” also apply to tissues/non-human animals comprising or derived from these cells.

The (biological) sample or composition, comprising a plurality of candidate molecules are usually subject to a first screen. The samples/compositions tested positive in the first screen are often subject to subsequent screens in order to verify the previous findings and to select the most potent agonists/activators of alkylglycerol monooxygenase TMEM195. Upon multiple screening and selection rounds those candidate molecules will be selected which show a pronounced capacity to activate/agonize alkylglycerol monooxygenase TMEM195 as defined and disclosed herein. For example, batches (i.e. compositions/samples) containing many candidate molecules will be rescreened and batches with no or insufficient activatory activity of candidate molecules be discarded without re-testing.

For example, if a (biological) sample or composition with many different candidate molecules is tested and one (biological) sample or composition is tested positive, then it is either possible in a second screening to screen, preferably after purification, the individual molecule(s) of the (biological) sample or composition. It may also be possible to screen subgroups of the (biological) sample or composition of the first screen in (a) subsequent screen(s). The screening of compositions with subgroups of those candidate molecules tested in previous screening rounds will thus narrow in on (an) potential potent alkylglycerol monooxygenase TMEM 195 activator(s). This may facilitate and accelerate the screening process in particular when a large number of molecules is screened. Accordingly, the cycle number of screening rounds is reduced compared to testing each and every individual candidate molecule in (a) first (and subsequent) screen(s) (which is, of course, also possible). Thus, depending on the complexity or the number of the candidate molecules, the steps of the screening method described herein can be performed several times until the (biological) sample or composition to be screened comprises a limited number, preferably only one substance which is indicative for the capacity of activating alkylglycerol monooxygenase TMEM195 or increasing the alkylglycerol monooxygenase TMEM195 activity wherein an increase in the activity of a functional alkylglycerol monooxygenase is expected to have medical implications as outlined above.

The term “increase in alkylglycerol monooxygenase TMEM195 activity” in step (b) of the screening method as described above means that the “activity of alkylglycerol monooxygenase TMEM195” is elevated upon contacting the cell, tissue, or non-human animal comprising alkylglycerol monooxygenase TMEM195 with the candidate molecule, preferably in comparison to a (control) standard or reference value, as defined herein wherein an increase in the activity of a functional alkylglycerol monooxygenase is expected to have medical implications as outlined above, i.e., e.g., in the treatment of male infertility (i.e. to elicit/induce male infertility), neurodegeneration or recent memory loss associated with Alzheimer's disease as already described in detail above.

As defined and disclosed herein, the term “comprising alkylglycerol monooxygenase TMEM195” refers not only to the alkylglycerol monooxygenase TMEM195 gene(s) or proteins known in the art and described herein. Also reporter constructs comprising a promoter and/or enhancer region of alkylglycerol monooxygenase TMEM195 can be used in the screening/identifying methods. Accordingly, the cell(s), tissue(s) and/or non-human animals used in the context of the present invention, in particular in context of the screening/identifying methods can comprise the reporter constructs disclosed herein and described below.

The activity of alkylglycerol monooxygenase TMEM195 can be quantified in cells, tissue or non-human animals.

The method for assessing the activity of a candidate molecule suspected of being an agonist/activator of alkylglycerol monooxygenase TMEM 195 can be accomplished by determining an increase in the activity of alkylglycerol monooxygenase TMEM195, wherein the increase in alkylglycerol monooxygenase TMEM195 activity can be detected with polynucleotides capable of hybridizing the alkylglycerol monooxygenase TMEM195 sense molecule. Alternatively, the increase in alkylglycerol monooxygenase TMEM195 activity can be detected with antibodies capable of binding the alkylglycerol monooxygenase TMEM195 protein. Therefore, the activity of alkylglycerol monooxygenase TMEM195 can be quantified by measuring, for example, the level of gene products (e.g. mRNA and/or protein of alkylglycerol monooxygenase TMEM195) by any of the herein described methods, activities, the alkylglycerol monooxygenase TMEM195 concentration or other cellular functions. As mentioned herein above the candidate compound to be tested may lead to a modified activity of alkylglycerol monooxygenase TMEM195 and an increase in the alkylglycerol monooxygenase TMEM195 activity is indicative for the capacity to agonize alkylglycerol monooxygenase TMEM195 and thus to to have medical implications as outlined above, i.e., e.g., to elicit/induce male infertility and to ameliorate both the neurodegeneration and the recent memory loss associated with Alzheimer's disease.

As mentioned, a “increased alkylglycerol monooxygenase TMEM195 activity” and, accordingly, an increased concentration/amount of alkylglycerol monooxygenase TMEM195 proteins in a sample may be reflected in a increased expression of the corresponding gene(s) encoding the alkylglycerol monooxygenase TMEM 195 protein(s). Therefore, a quantitative assessment of the gene product (e.g. protein or spliced, unspliced or partially spliced mRNA) can be performed in order to evaluate decreased expression of the corresponding gene(s) encoding the alkylglycerol monooxygenase TMEM195 protein(s). Also here, a person skilled in the art is aware of standard methods to be used in this context or may deduce these methods from standard textbooks (e.g. Sambrook, 2001, loc. cit.). For example, quantitative data on the respective concentration/amounts of mRNA from alkylglycerol monooxygenase TMEM195 can be obtained by Northern Blot, Real Time PCR and the like. Preferably, the concentration/amount of the gene product (e.g. the herein above described alkylglycerol monooxygenase TMEM195 mRNA or alkylglycerol monooxygenase TMEM195 protein) may be increased by at least about 10%, 20%, 30%, 40%, preferably by at least 50%, 60%, 70%, 80%, 90%, or 100% compared to a control sample. It is preferred herein that alkylglycerol monooxygenase TMEM195 proteins are (biologically) active or functional. Methods for determining the activity of alkylglycerol monooxygenase TMEM195 are described herein above and below. Since the alkylglycerol monooxygenase TMEM195 proteins are preferably (biologically) active/functional (wherein it is preferred that at least 70%, 75%, preferably at least 80%, 85%, 90%, 95%, 96, %, 97%, 98% and most preferably, at least 99% of alkylglycerol monooxygenase TMEM195 proteins of a sample a (biologically) active/functional), an increased concentration/amount of alkylglycerol monooxygenase TMEM 195 proteins in a sample reflects a increased (biological) activity of the alkylglycerol monooxygenase TMEM195 protein.

As mentioned, a person skilled in the art is aware of standard methods to be used for determining or quantitating expression of a nucleic acid molecule encoding, for example, the alkylglycerol monooxygenase TMEM195 (or fragments thereof). For example, the expression can be determined on the protein level by taking advantage of immunoagglutination, immunoprecipitation (e.g. immunodiffusion, immunelectrophoresis, immune fixation), western blotting techniques (e.g. (in situ) immuno histochemistry, (in situ) immuno cytochemistry, affinitychromatography, enzyme immunoassays), and the like. Amounts of purified polypeptide in solution can be determined by physical methods, e.g. photometry. Methods of quantifying a particular polypeptide in a mixture rely on specific binding, e.g of antibodies. Specific detection and quantitation methods exploiting the specificity of antibodies comprise for example immunohistochemistry (in situ). For example, concentration/amount of alkylglycerol monooxygenase TMEM195 proteins in a cell, tissue or a non-human animal can be determined by enzyme linked-immunosorbent assay (ELISA). Alternatively, Western Blot analysis or immunohistochemical staining can be performed. Western blotting combines separation of a mixture of proteins by electrophoresis and specific detection with antibodies. Electrophoresis may be multi-dimensional such as 2D electrophoresis. Usually, polypeptides are separated in 2D electrophoresis by their apparent molecular weight along one dimension and by their isoelectric point along the other direction. Expression can also be determined on the nucleic acid level (e.g. if the gene product/product of the coding nucleic acid sequence is an unspliced/partially spliced/spliced mRNA) by taking advantage of Northern blotting techniques or PCR techniques, like in-situ PCR or Real time PCR. Quantitative determination of mRNA can be performed by taking advantage of northern blotting techniques, hybridization on microarrays or DNA chips equipped with one or more probes or probe sets specific for mRNA transcripts or PCR techniques referred to above, like, for example, quantitative PCR techniques, such as Real time PCR.

These and other suitable methods for detection and/or determination of the concentration/amount of (specific) mRNA or protein(s)/polypeptide(s) are well known in the art and are, for example, described in Sambrook (2001), loc. cit.).

A skilled person is capable of determining the amount of mRNA or polypeptides/proteins, in particular the gene products described herein above, by taking advantage of a correlation, preferably a linear correlation, between the intensity of a detection signal and the amount of, for example, the mRNA or polypeptides/proteins to be determined.

The difference, as disclosed herein is statistically significant and a candidate molecule(s) is (are) selected, if the alkylglycerol monooxygenase TMEM195 activity (or of a corresponding reporter signal) is strongly increased. For example, the alkylglycerol monooxygenase TMEM195 activity (or of a corresponding reporter signal) may be increased by at least 50%, 60%, 70%, 80%, more preferably by at least 90% compared to the (control) standard value. In a cell based method the cells can be transfected with one or more constructs encoding alkylglycerol monooxygenase TMEM195 or a functional fragment thereof.

Preferably, the selected compound has a high alkylglycerol monooxygenase TMEM195 activating/agonizing activity. This can be reflected in the capacity of the alkylglycerol monooxygenase TMEM195 agonist/activator to potently increase the activity of alkylglycerol monooxygenase TMEM 195.

The above detected difference between the activity of alkylglycerol monooxygenase TMEM195 or the activity of a functional fragment of alkylglycerol monooxygenase TMEM195 in a cell, tissue or a non-human animal contacted with said candidate molecule and the activity in the (control) standard value (measured e.g. in the absence of said candidate molecule) may be reflected by the presence, the absence, the increase or the decrease of a specific signal in the readout system.

Genetic readout systems are also envisaged. Analogously, the activity of alkylglycerol monooxygenase TMEM195 or of a functional fragment thereof may be quantified by any molecular biological method as described herein. A skilled person is also aware of standard methods to be used in determining the amount/concentration of alkylglycerol monooxygenase TMEM195 expression products (in particular the protein and the nucleic acid level of alkylglycerol monooxygenase TMEM195) in a sample or may deduce corresponding methods from standard textbooks (e.g. Sambrook, 2001).

Also in this context, reporter constructs comprising a promoter and/or enhancer region of alkylglycerol monooxygenase TMEM195 can be used in the screening/identifying methods. Exemplary reporters (preferably associated with the reporter signals disclosed herein) are luciferase and fluorescent proteins, like GFP, RFP and the like. It is preferred that a promoter and/or enhancer element/region of alkylglycerol monooxygenase TMEM195 is used in this context and is fused to a reporter.

Exemplary reporter signals, reporters and reporter constructs are described herein below. Interesting reporters, namely reporter gene products, which can be used in the screening and identifying methods of the invention like luciferase, (green/red) fluorescent protein and variants thereof, EGFP (enhanced green fluorescent protein), RFP (red fluorescent protein, like DsRed or DsRed2), CFP (cyan fluorescent protein), BFP (blue green fluorescent protein), YFP (yellow fluorescent protein), (3-galactosidase or chloramphenicol acetyltransferase as well as methods for their detection are also described herein below in detail. Luciferase is a well known reporter; see, for example, Jeffrey (1987) Mol. Cell. Biol. 7(2), 725-737. A person skilled in the art can easily deduce further luciferase nucleic and amino acid sequences to be used in context of the present invention from corresponding databases and standard text books/review.

Further exemplary reporter constructs to be employed in context of the present invention, in particular the screening and identifying methods, comprise promoter(s) (and/or (a) enhancer region(s)) of alkylglycerol monooxygenase TMEM195, wherein the (initiation/enhancement of the) expression of the reporter(s) is under control of the promoter and/or enhancer of alkylglycerol monooxygenase TMEM195. A skilled person may easily retrieve these and other well-known sequences from databases (like NCBI) and use these sequences in the generation of reporter constructs to be employed herein. For example, the alkylglycerol monooxygenase TMEM 195 promoter sequence the corresponding database.

These reporter constructs comprising a reporter and a promoter (and/or enhancer) as defined above, are particularly useful in screening methods and assays, since the reporter signal associated with the reporter can easily be detected. A change in the reporter signal is indicative for the capacity of a candidate molecule tested to act as a agonist/activator of alkylglycerol monooxygenase TMEM195. For example, an agonist of alkylglycerol monooxygenase TMEM195 will lead to an increase of a reporter signal/activity of a reporter under control of the alkylglycerol monooxygenase TMEM195 promoter region. In particular, a reporter construct may comprise a luciferase gene and a promoter of alkylglycerol monooxygenase TMEM195. A person skilled in the art is easily in the position to generate this and other reporter constructs using routine techniques. Inter alia, vectors such as the pRL-TK RENILLA Vector and other well known vectors may be employed in the generation of the reporter constructs.

Apparently, increased expression of the reporter gene/activity of the reporter gene product will reflect a increased alkylglycerol monooxygenase TMEM195 activity, in particular an increased concentration/amount of alkylglycerol monooxygenase TMEM 195 protein. Alternatively, the effect of the agonist/activator on the expression of (a) reporter gene(s) may be evaluated by determining the amount/concentration of the gene product of the reporter gene(s) (e.g. protein or spliced, unspliced or partially spliced mRNA). Further methods to be used in the assessment of mRNA expression of a reporter gene are within the scope of a skilled person and also described herein below.

As mentioned above, it is preferred that a promoter and/or enhancer element/region of alkylglycerol monooxygenase TMEM195 is used in this context and is fused to a reporter. As used herein, the term “reporter construct for alkylglycerol monooxygenase TMEM195-inhibition” relates to any biotechnologically engineered construct allowing the detection of alkylglycerol monooxygenase TMEM195 activation. Accordingly, said reporter construct may allow the detection of alkylglycerol monooxygenase TMEM195-activation by inducing a change in the signal strength of a detectable signal. Said detectable signal may be selected from the group consisting of, but not limited to a fluorescence resonance energy transfer (FRET) signal, a fluorescence polarization (FP) signal and a scintillation proximity (SP) signal. In a further embodiment, said detectable signal may be associated with a reporter gene product. Examples of reporter gene products include luciferase, (green/red) fluorescent protein and variants thereof, like EGFP (enhanced green fluorescent protein), RFP (red fluorescent protein, like DsRed or DsRed2), CFP (cyan fluorescent protein), BFP (blue green fluorescent protein), YFP (yellow fluorescent protein), β-galactosidase or chloramphenicol acetyltransferase, and the like. For example, GFP can be derived from Aequorea victoria (U.S. Pat. No. 5,491,084). A plasmid encoding the GFP of Aequorea victoria is available from the ATCC Accession No. 87451. Other mutated forms of this GFP including, but not limited to, pRSGFP, EGFP, RFP/DsRed, DSRed2, and EYFP, BFP, YFP, among others, are commercially available from, inter alia, Clontech Laboratories, Inc. (Palo Alto, Calif.).

In another preferred embodiment, the non-human animal comprising said reporter construct for detecting alkylglycerol monooxygenase TMEM195 activation is a transgenic non-human animal. The non-human organism to be used in the described screening assays is preferably selected from the group consisting of C. elegans, yeast, drosophila, zebrafish, guinea pig, rat and mouse. The generation of such a transgenic animal is within the skill of a skilled artisan. Corresponding techniques are, inter alia, described in “Current Protocols in Neuroscience” (2001), John Wiley&Sons, Chapter 3.16. Accordingly, the invention also relates to a method for the generation of a non-human transgenic animal comprising the step of introducing a reporter construct for detecting alkylglycerol monooxygenase TMEM195 activation as disclosed herein into an ES-cell or a germ cell. The non-human transgenic animal provided and described herein is particular useful in screening methods and pharmacological tests described herein above. In particular the non-human transgenic animal described herein may be employed in drug screening assays as well as in scientific and medical studies wherein agonists/activators of alkylglycerol monooxygenase TMEM195 for the treatment of a disease related to male infertility, neurodegeneration or the recent memory loss associated with Alzheimer's disease are tracked, selected and/or isolated.

The transgenic/genetically engineered cell(s), tissue(s), and/or non-human animals to be used in context of the present invention, in particular, the screening/identifying methods, preferably comprise the herein described and defined reporter constructs. Hence, in this context, reporter constructs may comprise a promoter and/or enhancer region of alkylglycerol monooxygenase TMEM195 as defined herein. Exemplary reporters (preferably associated with the reporter signals disclosed herein) are luciferase and fluorescent proteins, like GFP, RFP and the like. Exemplary, non-limiting constructs to be used may comprise a luciferase reporter under control of a (human) alkylglycerol monooxygenase TMEM195 promoter and/or enhancer region. Exemplary reporters are luciferase and fluorescent proteins, like GFP, RFP and the like.

The method for assessing the activity of a candidate molecule suspected of being an agonists/activators of alkylglycerol monooxygenase TMEM195 can be accomplished by determining an increase in the activity of alkylglycerol monooxygenase TMEM195, wherein the increase in alkylglycerol monooxygenase TMEM195 activity can be detected with polynucleotides capable of hybridizing the alkylglycerol monooxygenase TMEM195 sense molecule.

In a preferred embodiment, the method for assessing the activity of a candidate molecule suspected of being an agonist/activator of alkylglycerol monooxygenase TMEM 195 can be accomplished by determining an increase in the activity of alkylglycerol monooxygenase TMEM 195, wherein the increase in alkylglycerol monooxygenase TMEM 195 activity can be detected by monitoring the enzyme activity of the alkylglycerol monooxygenase TMEM195 itself, wherein an increase of the alkylglycerol monooxygenase TMEM195 activity is indicative for the capacity of the selected molecule to to have medical implications as outlined above, i.e., e.g., induce/elicit male infertility and to ameliorate both the neurodegeneration and the recent memory loss associated with Alzheimer's disease. A cell to be used is a cell that comprises and expresses alkylglycerol monooxygenase TMEM195. The alkylglycerol monooxygenase TMEM195 activity can be quantified in cells, tissue or non-human animals can be assayed as already described in detail above and in the appended examples.

In a preferred embodiment, the method for assessing the activity of a candidate molecule suspected of being an activator/agonist of alkylglycerol monooxygenase TMEM195 can be accomplished by determining the physical interaction/binding of candidate molecules with alkylglycerol monooxygenase TMEM195. Interaction methods are known in the art. Interaction assays employing read-out systems are well known in the art and comprise, inter alia, two hybrid screenings (as, described, inter alia, in EP-0 963 376, WO 98/25947, WO 00/02911), GST-pull-down columns, co-precipitation assays from cell extracts as described, inter alia, in Kasus-Jacobi (2000) Oncogene 19:2052-2059, “interaction-trap” systems (as described, inter alia, in U.S. Pat. No. 6,004,746) expression cloning (e.g. lamda gtII), phage display (as described, inter alia, in U.S. Pat. No. 5,541,109), in vitro binding assays and the like. Further interaction assay methods and corresponding read out systems are, inter alia, described in U.S. Pat. No. 5,525,490, WO 99/51741, WO 00/17221, WO 00/14271, WO 00/05410 or Yeast Four hybrid assays as described in Sandrok (2001) JBC 276:35328-35333.

Said interaction assays for alkylglycerol monooxygenase TMEM195 also comprise assays for FRET-assays, TR-FRETs (in “A homogenius time resolved fluorescence method for drug discovery” in: High throughput screening: the discovery of bioactive substances. Kolb (1997) J. Devlin. NY, Marcel Dekker 345-360) or commercially available assays, like “Amplified Luminescent Proximity Homogenous Assay”, BioSignal Packard. Furthermore, the yeast-2-hybrid (Y2H) system may be employed to elucidate further particular and specific interaction, association partners of alkylglycerol monooxygenase TMEM195. Said interaction/association partners are suspected of being an agonist/activator of alkylglycerol monooxygenase TMEM195 and are further screened for their agonistic/activatory effects as described above. As mentioned herein above, the candidate compound that interacts with alkylglycerol monooxygenase TMEM195 may lead to a modified activity of alkylglycerol monooxygenase TMEM195 and an increase in the alkylglycerol monooxygenase TMEM195 activity is indicative for the capacity to agonize alkylglycerol monooxygenase TMEM195 and thus to have medical implications as outlined above, e.g., to elicit male infertility and to ameliorate both the neurodegeneration and the recent memory loss associated with Alzheimer's disease.

Similarly, interacting molecules (for example) (poly)peptides may be deduced by cell-based techniques well known in the art. These assays comprise, inter alia, the expression of reporter gene constructs or “knock-in” assays, as described, for, e.g., the identification of drugs/small compounds influencing the (gene) expression of alkylglycerol monooxygenase TMEM195. Said “knock-in” assays may comprise “knock-in” of alkylglycerol monooxygenase TMEM195 (or (a) fragment(s) thereof) in tissue culture cells, as well as in (transgenic) animals. Examples for successful “knock-ins” are known in the art (see, inter alia, Tanaka (1999) Neurobiol. 41:524-539 or Monroe (1999) Immunity 11:201-212). Furthermore, biochemical assays may be employed which comprise, but are not limited to, binding of the alkylglycerol monooxygenase TMEM195 (or (a) fragment(s) thereof) to other molecules/(poly)peptides, peptides or binding of the alkylglycerol monooxygenase TMEM195 (or (a) fragment(s) thereof) to itself (themselves) (dimerizations, oligomerizations, multimerizations) and assaying said interactions by, inter alia, scintillation proximity assay (SPA) or homogenous time-resolved fluorescence assay (HTRFA).

Said “testing of interaction” may also comprise the measurement of a complex formation. The measurement of a complex formation is well known in the art and comprises, inter alia, heterogeneous and homogeneous assays. Homogeneous assays comprise assays wherein the binding partners remain in solution and comprise assays, like agglutination assays. Heterogeneous assays comprise assays like, inter alia, immuno assays, for example, ELISAs, RIAs, IRMAs, FIAs, CLIAs or ECLs.

The interaction of the agonistic molecules of alkylglycerol monooxygenase TMEM195 mRNA and alkylglycerol monooxygenase TMEM195 protein or fragments thereof may also be tested by molecular biological methods, like two-, three- or four-hybrid-assays, RNA protection assays, Northern blots, Western blots, micro-, macro- and protein- or antibody arrays, dot blot assays, in situ hybridization and immunohistochemistry, quantitative PCR, coprecipitation, far western blotting, phage based expression cloning, surface plasmon resonance measurements, yeast one hybrid screening, DNAse I, footprint analysis, mobility shift DNA-binding assays, gel filtration chromatography, affinity chromatography, immunoprecipitation, one- or two dimensional gel electrophoresis, aptamer technologies, as well as high throughput synthesis and screening methods.

In sum, the present invention provides for the first time methods for identifying, and characterizing (a) candidate molecule(s) or (a) compound(s) which are capable of agonizing/activating alkylglycerol monooxygenase TMEM195 whereby said inhibition may lead to an increase in the activity of a functional alkylglycerol monooxygenase is expected to have medical implications as outlined above, i.e., e.g., to induce male infertility and to ameliorate both the neurodegeneration and the recent memory loss associated with Alzheimer's disease. Therefore the present invention provides for screening as well as identification methods for agonists of alkylglycerol monooxygenase TMEM195. As also disclosed herein above, the term “agonist” relates to molecules or compounds that bind to alkylglycerol monooxygenase TMEM195 or a functional fragment thereof, thereby activating and/or increasing alkylglycerol monooxygenase TMEM195 activity, wherein these alkylglycerol monooxygenase TMEM195 agonists elicit male infertility and ameliorate both the neurodegeneration and the recent memory loss associated with Alzheimer's disease.

In line with the above, the present invention also relates in a further embodiment to the method of the invention as outlined above, wherein the decrease or increase in alkylglycerol monooxygenase activity is detected with polynucleotides capable of hybridizing the alkylglycerol monooxygenase sense molecule or with antibodies as defined above.

In a further preferred embodiment, in accordance with the above and in relation with the embodiments of this invention, the present invention relates also to a pharmaceutical composition comprising the antagonists/inhibitors or agonists/activators of alkylglycerol monooxygenase TMEM195 as selected in the above-defined screening method for assessing the activity of a candidate molecule suspected of being an antagonist/inhibitor of alkylglyerol monooxygenase (TMEM 195; glyceryl ether monooxygenase; EC 1.14.16.5) of for assessing the activity of a candidate molecule suspected of being an agonist/activator of alkylglyerol monooxygenase (TMEM195; glyceryl ether monooxygenase; EC 1.14.16.5). Such pharmaceutical composition may as already described above, inter alia, be used in treating cancer hypertension or cateract (for antagonists/inhibitors of alkylglyerol monooxygenase TMEM195) or be used in treating male infertility and to ameliorate both the neurodegeneration and the recent memory loss associated with Alzheimer's disease (for agonist/activator of alkylglyerol monooxygenase TMEM195). The embodiments disclosed in the context with the pharmaceutical compositions as defined above apply, mutatis mutandis, to the pharmaceutical compositions comprising the antagonists/inhibitors or agonists/activators of alkylglycerol monooxygenase TMEM195 as selected in the above-defined screening method.

In a further embodiment, the present invention relates to the use of a cell, tissue or a non-human animal of the invention for screening and/or validation of a compound suspected of being an antagonist/inhibitor or agonist/activator of alkylglycerol monooxygenase. The term “cell” as used in this context may also comprise a plurality of cells as well as cells comprised in a tissue. A cell to be used is a cell that comprises and expresses alkylglycerol monooxygenase TMEM195. Cells, tissues and non-human animals to be used in accordance with the present invention are also described herein above.

The used non-human animal or cell may be transgenic or non transgenic. In this context the term “transgenic” particularly means that at least one of the alkylglycerol monooxygenase TMEM195 gene as described herein is overexpressed, thus the alkylglycerol monooxygenase TMEM195 activity in the non-human transgenic animal or a transgenic animal cell is enhanced. Generally, it is preferred herein that alkylglycerol monooxygenase TMEM195 is highly expressed in (a) cell(s), tissue(s), non-human animal to be used in the screening methods as described above.

The term “transgenic non-human-animal”, “transgenic cell” or “transgenic tissue” as used herein refers to an non-human animal, tissue or cell, not being a human that comprises different genetic material of a corresponding wild-type animal, tissue or cell. The term “genetic material” in this context may be any kind of a nucleic acid molecule, or analogues thereof, for example a nucleic acid molecule, or analogues thereof as defined herein. The term “different” means that additional or fewer genetic material in comparison to the genome of the wild type animal or animal cell. An overview of different expression systems to be used for generating transgenic cell/animal refers for example to Methods in Enzymology 153 (1987), 385-516, in Bitter et at (Methods in Enzymology 153 (1987), 516-544) and in Sawers et al. (Applied Microbiology and Biotechnology 46 (1996), 1-9), Billman-Jacobe (Current Opinion in Biotechnology 7 (1996), 500-4), Hockney (Trends in Biotechnology 12 (1994), 456-463), Griffiths et al., (Methods in Molecular Biology 75 (1997), 427-440).

In a preferred embodiment, the (transgenic) non-human animal or (transgenic) cell is or is derived from a mammal. Non-limiting examples of the (transgenic) non-human animal or derived (transgenic) cell are selected from the group consisting of a mouse, a rat, a rabbit, a guinea pig and Drosophila.

Generally, the (transgenic) cell may be a eukaryotic cell. For example, the (transgenic) cell in accordance with the present invention may be but is not limited to yeast, fungus, plant or animal cell. In general, the transformation or genetically engineering of a cell with a nucleic acid construct or a vector can be carried out by standard methods, as for instance described in Sambrook and Russell (2001), Molecular Cloning: A Laboratory Manual, CSH Press, Cold Spring Harbor, N.Y., USA; Methods in Yeast Genetics, A Laboratory Course Manual, Cold Spring Harbor Laboratory Press, 1990.

In a further embodiment, the present invention relates to a kit for carrying out the methods of assessing the activity of a candidate molecule suspected of being an antagonist/inhibitor or an agonist/activator of alkylglycerol monooxygenase as described above or for carrying out the methods of assessing the activity of a candidate molecule suspected of being an agonist/activator or agonist/activator of alkylglycerol monooxygenase as described above comprising, inter alia, polynucleotides and/or antibodies capable of detecting the activity of alkylglycerol monooxygenase. The embodiments disclosed in this context with the method of the present application apply, mutatis mutandis, to the kit of the present invention.

Advantageously, the kit of the present invention further comprises, optionally (a) reaction buffers, storage solutions, wash solutions and/or remaining reagents or material required in the pharmacological and drug screening assays or the like as described herein. Furthermore, parts of the kit of the invention can be packed individually in vials or bottles or in combination in containers or multicontainer units.

In a preferred embodiment of the present invention, the kit may be advantageously used for carrying out the method for detecting the alkylglycerol monooxygenase TMEM195 activity or changes in the alkylglycerol monooxygenase TMEM195 activity as described herein. Additionally, the kit of the present invention may contain means for detection suitable for scientific, medical and/or diagnostic purposes. The manufacture of the kits follows preferably standard procedures which are known to the person skilled in the art.

Similarly, kits are provided which comprise the candidate molecule as described herein, the nucleic acid molecule, the cell, tissue or non-human animal of the invention. These kits provided herein are particularly useful in the methods of the present invention and in particular in the determination of the alkylglycerol monooxygenase TMEM195 activity or changes in the alkylglycerol monooxygenase TMEM195 activity. These kits as well as the methods provided herein are also useful in pharmacological screenings, also comprising “high-throughput” screening.

In a preferred embodiment, said kit comprises said vector, said recombinant host cell or the antibody as above-mentioned. Said kit may also comprise primers of the present invention. The polypeptides (or fragments thereof) of the present invention may also be comprised in kits. The kits of the present invention may be useful in diagnostic settings as well as in medical interventions. This kit can be used in diagnosis of identifying a condition wherein a decrease or an increase in the activity of a functional alkylglycerol monooxygenase is expected to have medical implications as outlined above. The kit of the present invention may comprise further components, like means of detection (like secondary antibodies, labeled antibodies). It may comprise negative or positive control samples. The kit (to be prepared in context) of this invention or the methods and uses of the invention may further comprise or be provided with (an) instruction manual(s), for example how carry out the diagnostic assays and methods provided herein, like the detection methods for allergic responses towards the antigens/allergens provided herein. Particularly, said instruction manual(s) may comprise guidance to use or apply the herein provided methods or uses. The kit (to be prepared in context) of this invention may further comprise substances/chemicals and/or equipment suitable/required for carrying out the methods and uses of this invention.

The following figures show and illustrate the present invention:

FIG. 1. Tetrahydrobiopterin-dependent enzymatic reactions in mammals.

(A) Names and reactions of tetrahydrobiopterin-dependent enzymes.

Official gene symbols, enzyme commission numbers and the enzymatic reactions with respect to the substrates are shown. All listed enzymes require tetrahydrobiopterin and molecular oxygen. One atom of the oxygen molecule is incorporated into the substrate, the other converted to water. For nitric oxide synthases, two of these mixed function oxygenase steps are required for conversion of one molecule of arginine to citrulline and NO. Nitric oxide synthases (blue) require heme for catalysis, the aromatic amino acid hydroxylases (black) contain a catalytically active non-heme iron, and, as inferred from sequence homology, alkylglycerol monooxygenase (red) uses a non-heme diiron center for catalysis.

(B) Phylogenetic tree of amino acids of tetrahydrobiopterin-dependent enzymes.

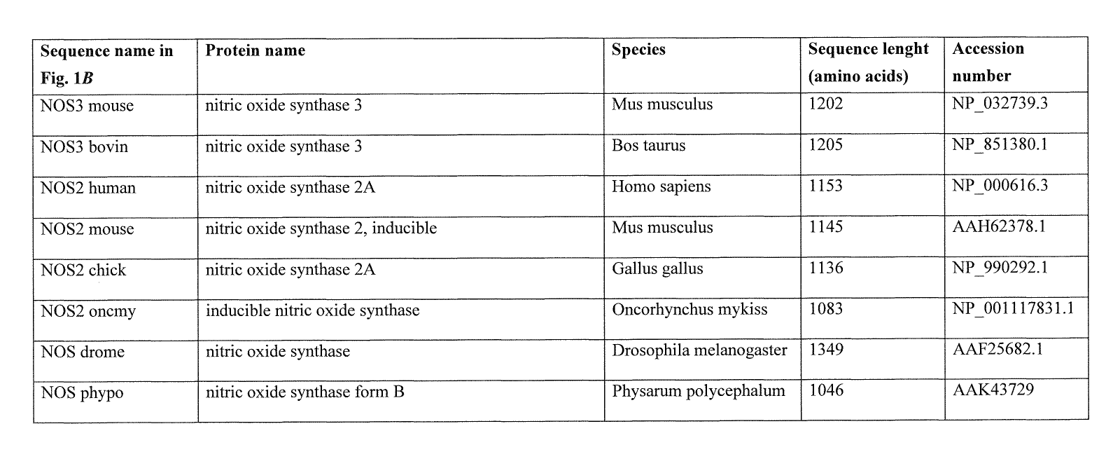

Representative protein sequences of each gene were selected from protein databases, aligned by ClustalW (Gonnet Matrix) and an UPMGA (unweighted pair group method using arithmetic averages) tree with Poisson correction drawn by MEGA4.0 using the default parameters (32). For details and accession numbers of protein sequences see FIG. 10. No sequence homology occurred across the three groups (shown in different colors) which differ in the form of iron required for catalysis. The scale bar gives a measure of the average fraction of amino acids exchanged between the sequences.

FIG. 2. Formulae of biologically active etherlipids.

FIG. 3. Degradation of alkylacylglycerolipids.

The acyl residue in position 2 of alkylacylglycerols (I) is first cleaved by a lipase type enzyme. This generates the corresponding “lyso” 1-O-alkylglycerolipids (II), which are substrates of alkylglycerol monooxygenase. No restrictions for R3 for substrates of alkylglycerol monooxygenase are known, the compounds may or may not be phospholipids, the hydroxy group at C3 may also be unsubstituted, C3 may even be missing, i.e. alkyl glycols are also substrates of the enzyme (20). The side chain at R1 may be 12 to 20 carbons total in length, but must not contain a double bond adjacent to the carbon bonded to the oxygen, i.e. plasmalogens are no substrates (14). In a tetrahydrobiopterin-dependent fashion, the first carbon of the alkyl side chain adjacent to the ether bond is hydroxylated, generating a hemiacetal (III) which decomposes to the corresponding glycerol derivative (IV) and a fatty aldehyde (V). Fatty aldehydes are toxic to cells and are oxidized by fatty aldehyde dehydrogenase to yield the less toxic corresponding fatty acid (VI). Tetrahydrobiopterin leaves the reaction as “quinoid” 6,7[8H]-dihydrobiopterin (14) (qH2biopterin) and is recycled to tetrahydrobiopterin by quinoid dihydropteridine reductase. Although it has not been demonstrated for alkylglycerol monooxygenase, the formation of 6,7[8H]-dihydrobiopterin from the initial enzymatic product formed from tetrahydrobiopterin may be facilitated in vivo by 4a-carbinolamine dehydratase (EC 4.2.1.96, PCBD1) like for aromatic amino acid hydroxylases (6).

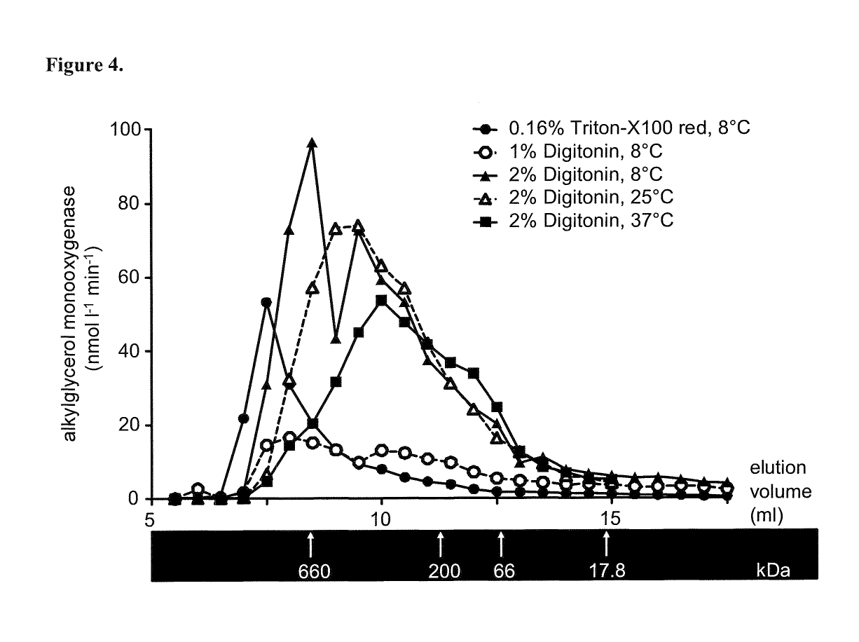

FIG. 4. Gel filtration of alkylglycerol monooxygenase solubilized from rat liver microsomes.

Microsomes were prepared from livers of male Sprague-Dawley rats (250-300 g body weight, kindly supplied by Helmut Prast, University of Innsbruck) as described (28). They were solubilized with Triton X-100 (reduced form, Fluka, Buchs, Switzerland) or with digitonin (Sigma) by gentle mixing for 45 min at the indicated temperatures, followed by centrifugation at 350,000×g for 30 min at 4° C. 200 μl were injected onto a Superose 12 300/10 GL column (GE Healthcare), eluted with 50 mM potassium phosphate, pH 7.5, 100 mM NaCl, 5% (v/v) glycerol and 0.1% (w/v) 3[(3-cholamidopropyedimethylammonio]-1-propanesulfonate (CHAPS, Sigma) at a flow rate of 0.5 ml/min and 4° C. 0.5 ml fractions were collected and alkylglycerol monooxygenase activity was determined as described (22). Molecular mass markers used for calibration were thyroglobuline (Calbiochem, Merck, Darmstadt, Germany; 660 kDa), 13-amylase (Sigma; 200 kDa), bovine serum albumin (Sigma; 66 kDa), equine myoglobin (Serva, Heidelberg, Germany; 17.8 kDa).

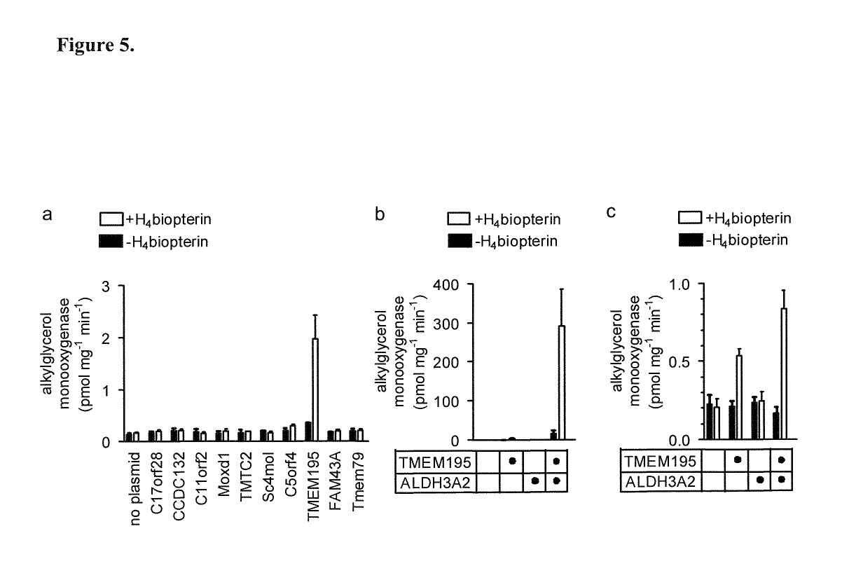

FIG. 5. Alkylglycerol monooxygenase activity generated by nucleic acids.

(A) Transfection of Chinese hamster ovary (CHO) cells with single expression plasmids.

80,000 CHO cells were transfected with 1 μg each of the indicated mammalian expression plasmids and cultured in presence of 1 μM of the tetrahydrobiopterin precursor sepiapterin for 24 hours. Alkylglycerol monooxygenase activity was quantified by incubation with a pyrene-labelled alkylglycerol and analysis of pyrenedecanoic acid by HPLC with fluorescence detection (22). Mean values of four measurements of three independent transfections±SEM are shown for measurements with tetrahydrobiopterin (H₄biopterin), values without tetrahydrobiopterin were determined in duplicate from two independent transfections.

(B) Cotransfection of CHO cells with TMEM195 and ALDH3A2. Experiments were performed as in (A), pairs of plasmids were supplied as 1 μg each. Mean values±SEM of 3-10 independent transfections are shown, each measurement was done with and without addition of tetrahydrobiopterin (H₄biopterin).

(C) Injection of Xenopus laevis oocytes with TMEM195 and ALDH3A2 cRNAs. 27-69 ng of capped, polyadenylated cRNA prepared from expression plasmids were injected into defolliculated Xenopus laevis oocytes. After 3-4 days in culture, oocytes were homogenized and alkylglycerol monooxygenase activity quantified by incubation with a pyrene-labelled alkylglycerol and analysis of pyrenedecanoic acid by HPLC with fluorescence detection (22). Mean values±SEM of 7-19 measurements on separately injected cRNAs are shown. Each measurement was done with and without addition of tetrahydrobiopterin (H₄biopterin).

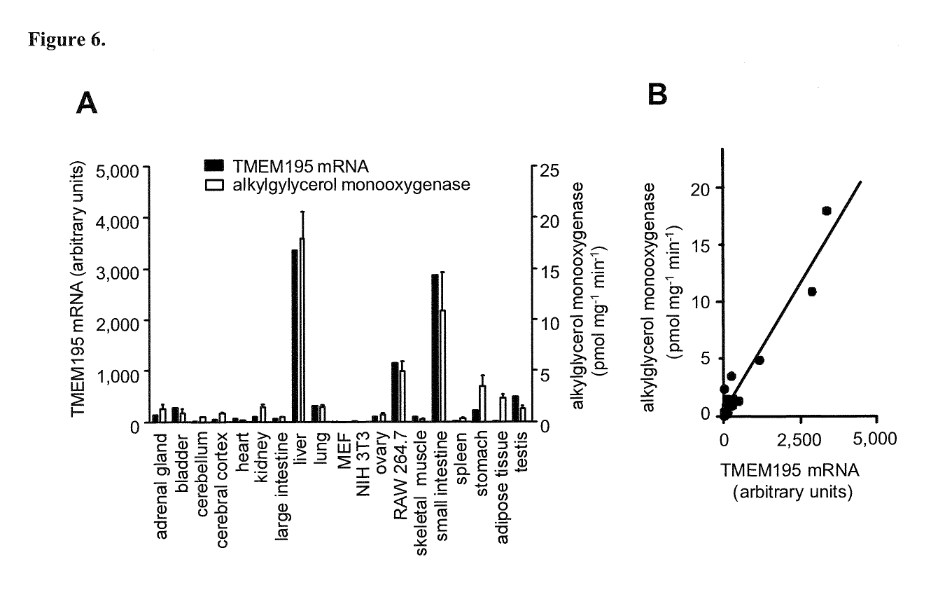

FIG. 6. Alkylglycerol monooxygenase activities and TMEM195 mRNA expression in mouse tissues and cells.

(A) Distribution of alkylglycerol monooxygenase activities and TMEM195 mRNA expression in mouse tissues and cells. Mouse tissues and cultured cells were homogenized, and alkylglycerol monooxygenase activities determined (22). Mean values of 4-6 independent measurements of tissues±SEM are shown. Enzyme activities were compared to publicly available gene expression data (BioGPS, http://biogps.gnf.org/). MEF stands for mouse embryonic fibroblasts.

(B) Correlation of alkylglycerol monooxygenase activities and TMEM195 mRNA expression in mouse tissues and cells. For every tissue and cell shown in (A), the alkylglycerol monooxygenase activity measured was plotted against TMEM195 mRNA expression data from BioGPS (http://biogps.gnf.org). Both parameters were significantly correlated (linear correlation, r²=0.93, p<0.0001; Spearman rank correlation, r=0.75, p=0.0002).

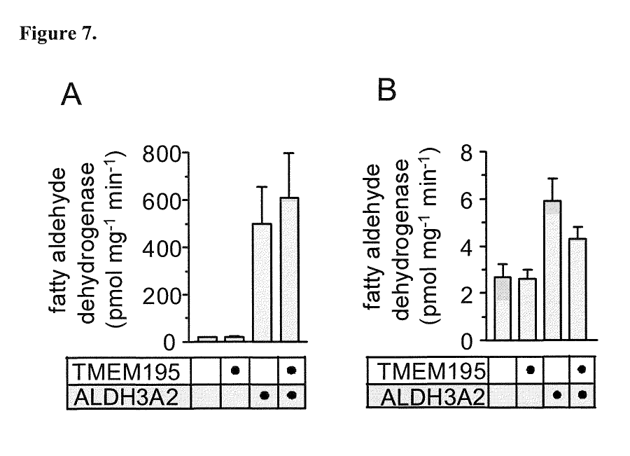

FIG. 7. Fatty aldehyde dehydrogenase activity generated by nucleic acids.

(A) Cotransfection of Chinese hamster ovary (CHO) cells with TMEM195 and/or ALDH3A2. 80,000 CHO cells were transfected with 1 μg each of the indicated mammalian expression plasmids and cultured in presence of 1 μM of the tetrahydrobiopterin precursor sepiapterin for 24 hours. Fatty aldehyde dehydrogenase activity was quantified in cell extracts by incubation with pyrenedecanal and analysis of pyrenedecanoic acid by HPLC with fluorescence detection (31). Mean values±SEM of 3-8 independent transfections are shown. ALDH3A2 transfection leads to strongly increased fatty aldehyde dehydrogenase activity (p<0.05).

(B) Injection of Xenopus laevis oocytes with TMEM195 and/or ALDH3A2 cRNAs. 27-69 ng of capped, polyadenylated cRNA prepared from expression plasmids were injected into defolliculated Xenopus laevis oocytes. After 3-4 days in culture, oocytes were homogenized and fatty aldehyde dehydrogenase activity was quantified by incubation with pyrenedecanal and analysis of pyrenedecanoic acid by HPLC with fluorescence detection (31). Mean values±SEM of 7-19 measurements of separately injected cRNAs are shown. Injections with ALDH3A2 lead to significantly increased fatty aldehyde dehydrogenase activities (p<0.05).

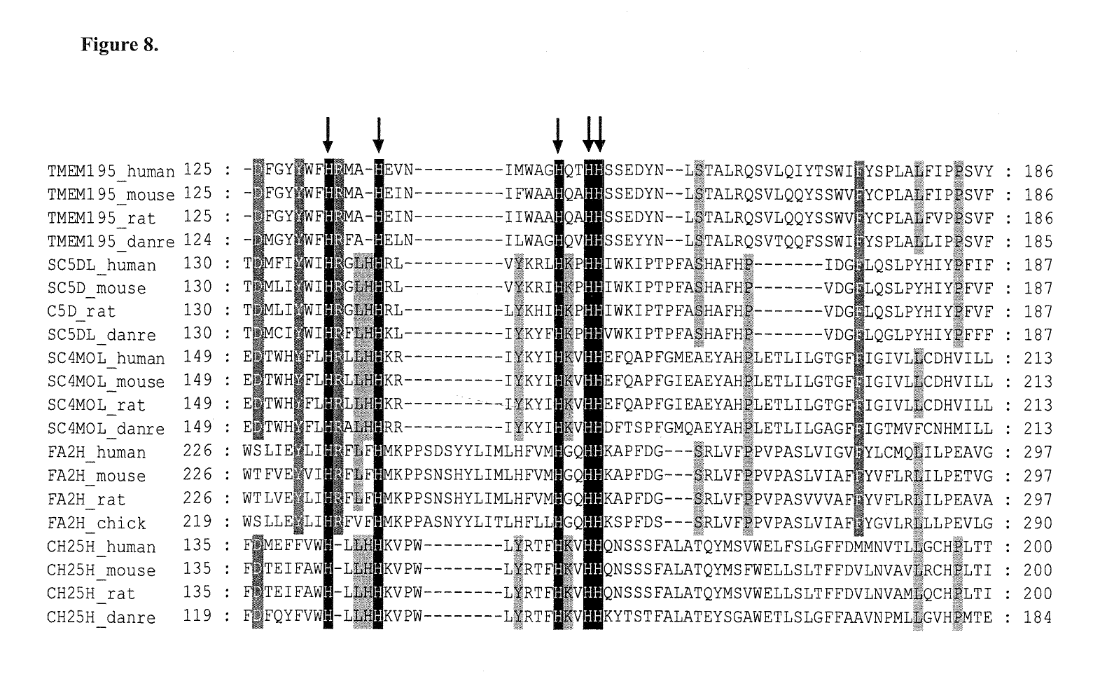

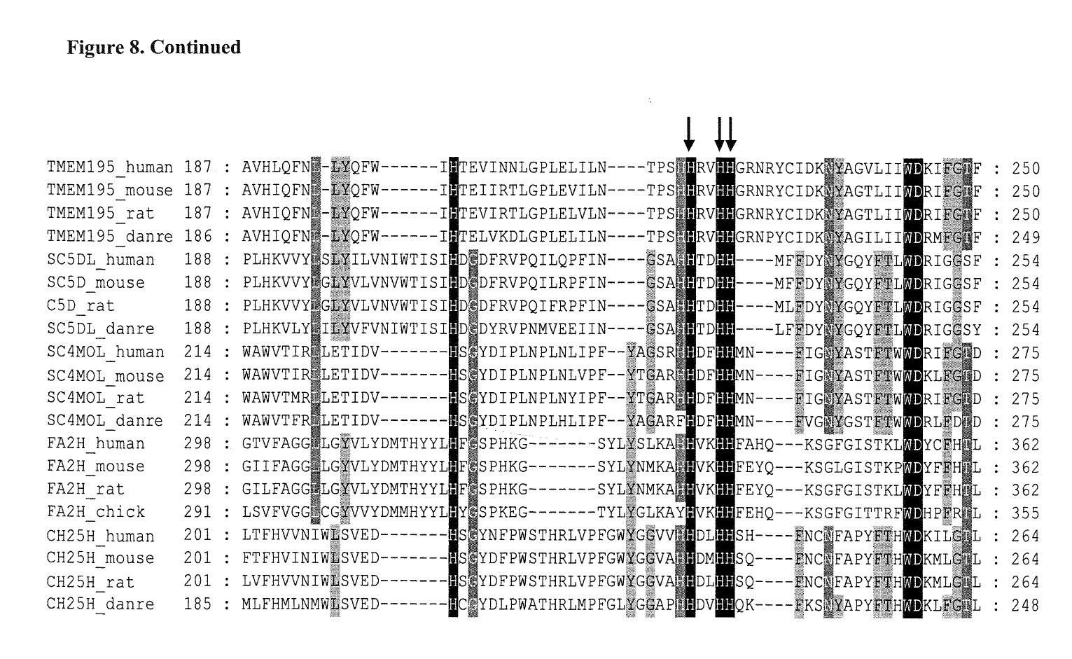

FIG. 8. Location of the eight conserved histidines in proteins containing the fatty acid hydroxylase motif.

Integral membrane fatty acid hydroxylases feature a characteristic motif of eight conserved histidines (33), HX(3,4)HX(7,41)HX(2,3)-HHX(61,189)[HQ]X(2,3)HH. Black arrows mark these eight histidines. The protein alignment was created by the ClustalW module of Mega 4.0 (32) using the identity option, all other parameters were used as default values. Text presentation and shading was done by genedoc (Nicholas K. B. and Nicholas H. B., http://www.psc.edu/biomed/genedoc). The motif is located in the loop (residues 132-333 for human TMEM195) between the second and third of five putative transmembrane regions (source for transmembrane regions: UniprotKB, http://www.uniprot.org/uniprot/). Accession numbers and lengths of sequences used were: Alkylglycerol monooxygenases: TMEM195_human, Q6ZNB7, 445 aa; TMEM195_mouse, Q8BS35, 447 aa; TMEM195_rat, AOJPQ8, 447 aa; TMEM195_danre, NP998048, 446 aa; Sterol-C5-desaturases: SC5DL_human, Q6GTM5, 299 aa; SC5D_mouse, Q8R1S3 299 aa; C5Drat, Q9EQS5, 299 aa; SCSDL_danre, NP_{—}001004630, 300 aa; sterol-C4-methyl oxidase-like: SC4MOL_human, Q15800, 293 aa; SC4MOL_mouse, Q9CRA4, 293 aa; SC4MOL_rat, 035532, 293 aa; SC4MOL_danre, NP_{—}998518, 291 aa; Fatty acid 2-hydroxylases: FA2H human, Q7L5A8, 372 aa; FA2H_Mouse, Q8BTH1, 372 aa; FA2H_rat, Q2LAM0, 372 aa; FA2H_chick, XP_{—}414053, 365 aa; Cholesterol 25-hydroxylases: CH25H human, NP_{—}003947, 272 aa; CH25H_mouse, Q9Z0F5, 298 aa; CH25H_rat, Q4QQV7, 298 aa; CH25H_danre, NP_{—}001008652, 251 aa.

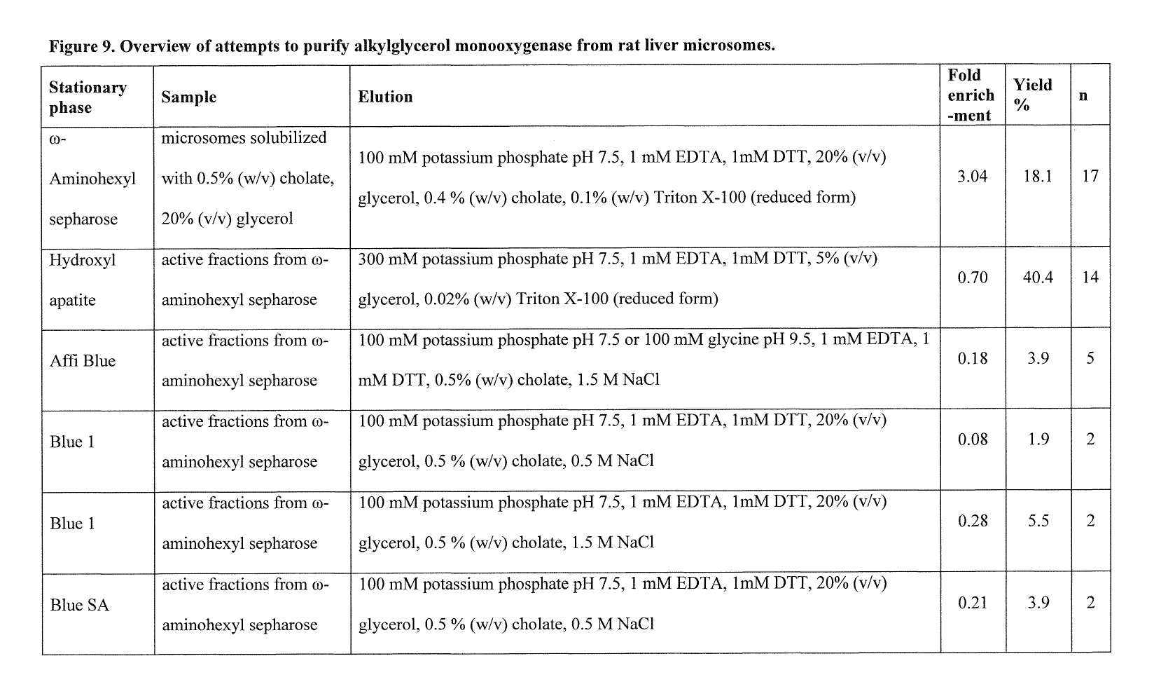

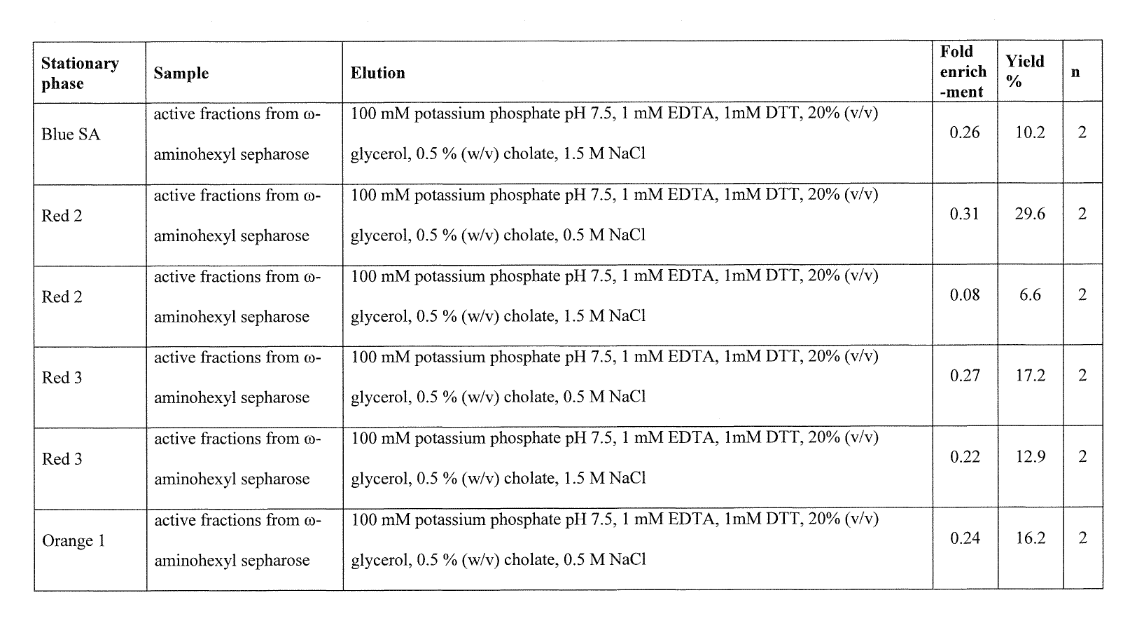

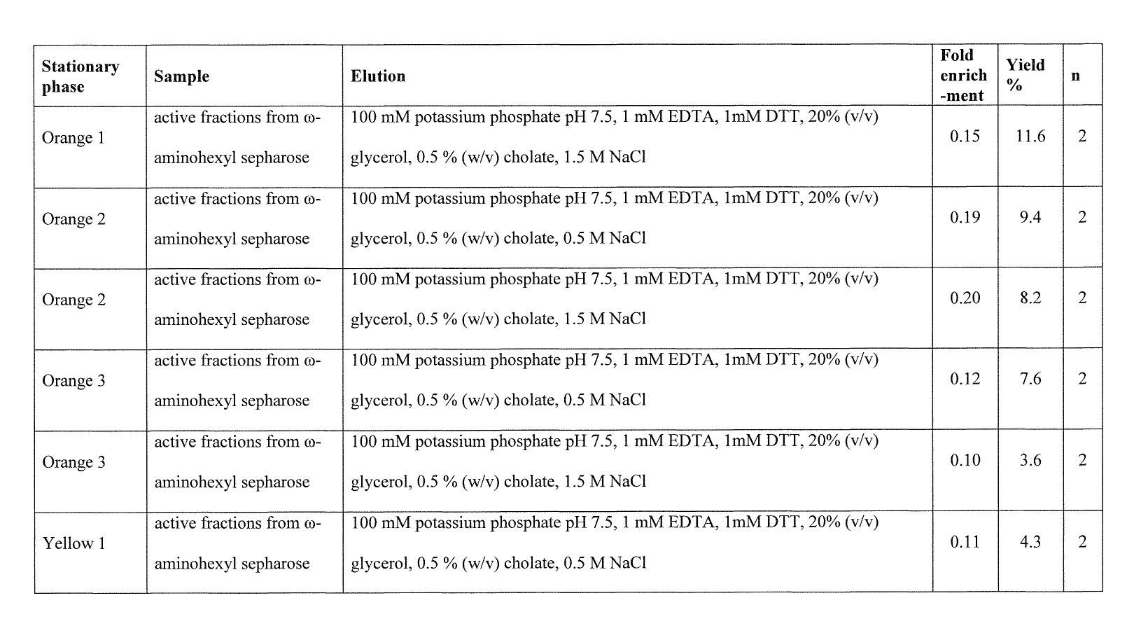

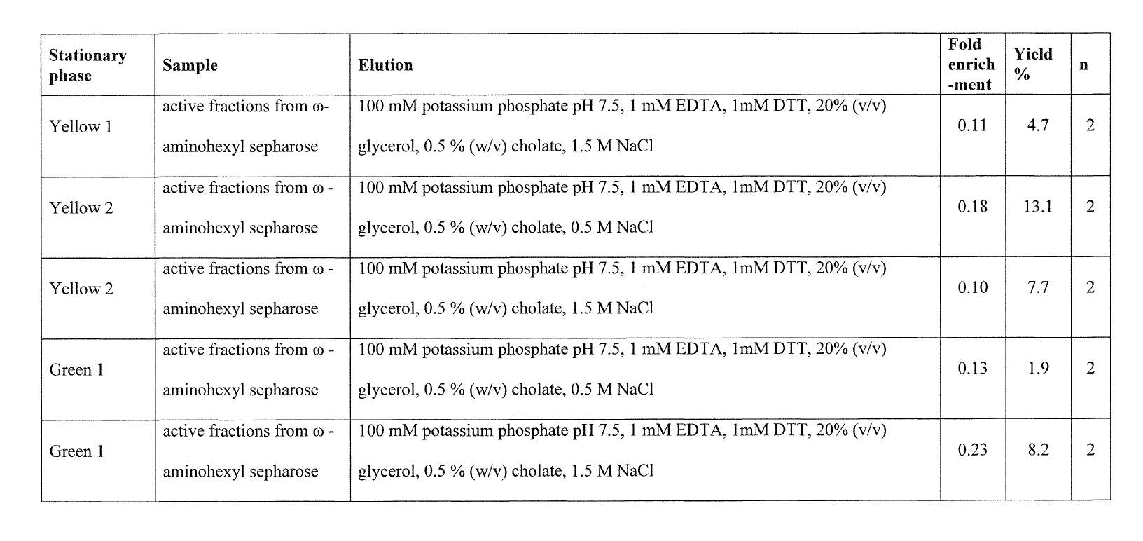

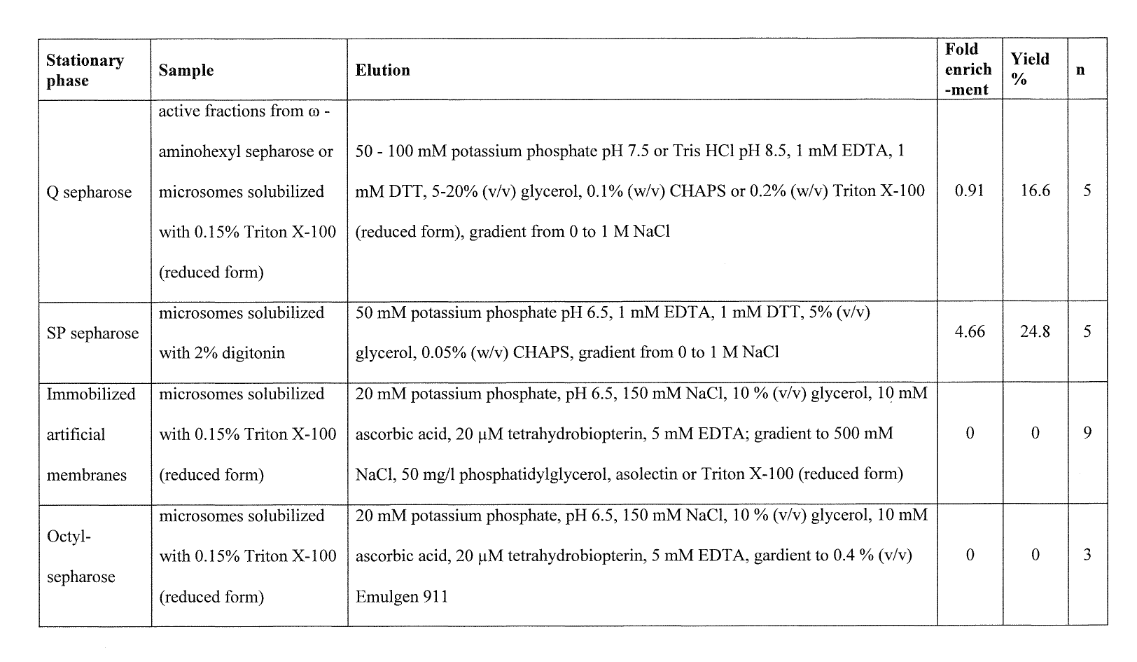

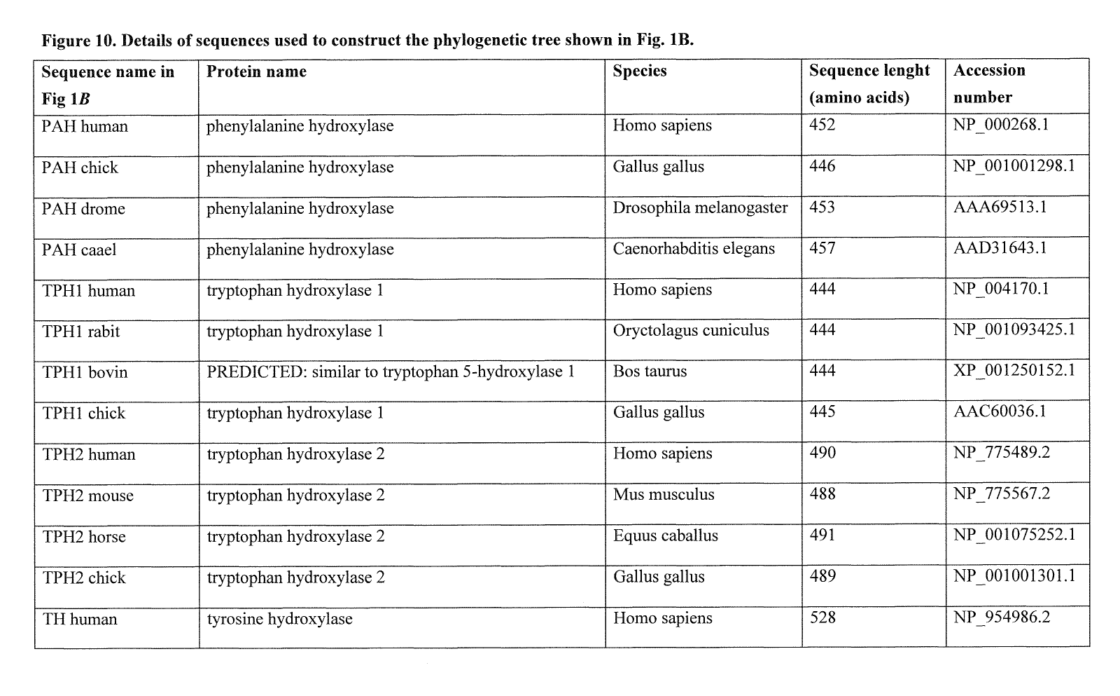

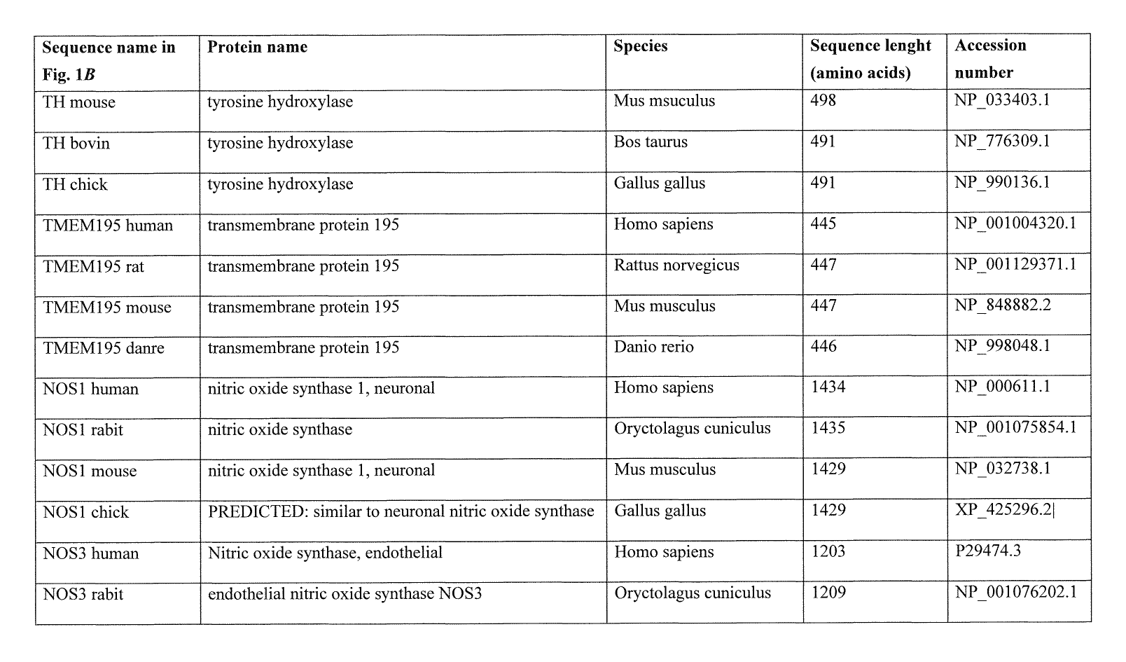

FIG. 9. Overview of attempts to purify alkylglycerol monooxygenase from rat liver microsomes.

All procedures were carried out at 4° C. Stationary phases were obtained from GE Healthcare, Sigma and ProMetic Biosciences (Cambridge, UK). The immobilized artificial membrane column was from Regis (Morton Grove, Ill., USA). For chromatography, a Titanium 1050 HPLC Hewlett Packard (Waldbronn, Germany) and a Merck-Hitachi L-5200 fraction collector, or a Pharmacia LKB Pump P1, an Uvicord SII and a fraction collector FRAC 100 (Pharmacia LKB, Uppsala, Sweden) were used. All fractions were assayed immediately for alkylglycerol monooxygenase activity using 1-O-pyrenedecylglycerol as substrate and quantification of pyrenedecanoic acid by HPLC with fluorescence detection (22). Protein was determined by the Bradford assay using bovine serum albumin as standard.

FIG. 10. Details of sequences used to construct the phylogenetic tree shown in FIG. 1B.

FIG. 10 lists the full protein names, the species, the lengths of the amino acid sequences, and the Genbank accession numbers of the sequences used to construct the phylogenetic tree shown in FIG. 1B.

The following non-limiting examples illustrate the invention:

As detailed exemplified in the examples below, the teaching of the examples is summarized in the following:

First, it has been tried to find sequence motifs characteristic for tetrahydrobiopterin-dependent reactions. This analysis, however, resulted in the conclusion that no protein sequence with significant homology to the tetrahydrobiopterin binding domain of aromatic amino acid hydroxylases can be found in databases (done in collaboration with Nicolas Hulo, Swiss Prot Team, Swiss Institute for Bioinformatics, Geneva). Then, it has been tried to construct a pattern matching the tetrahydrobiopterin binding region of aromatic amino acid hydroxylases and nitric oxide synthases based on the alignment presented by (34). It has been successfully constructed a pattern finding all animal aromatic amino acid hydroxylases and nitric oxide synthases, but no other proteins. Allowing one mismatch, this pattern picked C17orf28 from the databases. This clone, however, did not show activity (see below).

The core of the invention was the right choice of one of the more than 10.000 Pfam motifs that might occur in the alkylglycerol monooxygenase protein. The Pfam database is a large collection of protein families, each represented by multiple sequence alignments and hidden Markov models (HMMs). The current version of Pfam 24.0 (October 2009) contains 11912 families. Proteins are generally composed of one or more functional regions, commonly termed domains. Different combinations of domains give rise to the diverse range of proteins found in nature. The identification of domains that occur within proteins can therefore provide insights into their function (35).

It has been realized in the context of the present invention that the fatty acid hydroxylase motif (PF04116) describes a family of enzymes catalysing reactions that have some features in common with the alkylglycerol monooxygenase reaction, although none of these had been described to require tetrahydrobiopterin as cofactor. Along with human or murine expression plasmids containing the fatty acid hydroxylase motif, other expression plasmids resulting from alternative approaches have been tested, such as C17orf28 with the common tetrahydrobiopterin binding motif described above, or with a predicted tertiary structure with similarity to the crystal structure of phenylalanine hydroxylase.

When these expression plasmids were transfected to chinese hamster ovary (CHO) cells, only the one encoding for TMEM195 yielded alkylglycerol monooxygenase activity above background. This activity was strongly stimulated by cotransfection of fatty aldehyde dehydrogenase (ALDH3A2), the enzyme catalysing the reaction downstream of alkylglycerol monooxygenase. Like with the single plasmids, no combination of plasmids lacking TMEM195 yielded activity.

In the following, the invention is illustrated in detail by the non-limiting examples below:

### EXAMPLE I

### Materials and Methods as Employed in the Following

### Materials

1-O-pyrenedecyl-sn-glycerol was synthesized from glycerol and pyrenedecanol which was obtained from pyrenedecanoid acid by Vitride reduction as described (22). Recombinant rat fatty aldehyde dehydrogenase was obtained by E. coli expression of a Strep-tagged open reading frame obtained by polymerase chain reaction from a rat liver cDNA, and affinity purification (31). Recombinant Physarum polycephalum dihydropteridine reductase was expressed untagged in E. coli and purified as described (36). Pyrenedecanal was obtained from Ramidus AB, Lund, Sweden, pteridines were from Schircks, Jona Switzerland.

### Methods

### Mass Spectrometric Analysis of Partially Purified Alkylglycerol Monooxygenase Fractions.

Rat liver microsomes were solubilized with 0.5% (w/v) cholate and 20% (v/v) glycerol at 4° C., purified over ω-aminohexyl sepharose (Sigma, Vienna, Austria), active fractions pooled and purified over hydroxyl apatite (Sigma, see FIG. 9 for details). For three separate purifications, active fractions eluting from the hydroxylapatite column were analyzed for proteins differing from the inactive flowthrough by the Ettan DIGE system (GE Healthcare, Vienna, Austria). Briefly, fractions to be compared were minimally labelled with fluorescent dyes of three different colours (Cy2, Cy3 and Cy5, respectively), mixed, separated by two dimensional gel electrophoresis, protein spots analysed for differences using a three colour fluorescent scanner (Typhoon 9410, GE Healthcare) and evaluated using the Decycler software (GE Healthcare). Three times 30 μg of protein were separated using an 18 cm non-linear isoelectric focussing strip (pH 3-11, GE Healthcare) in the first dimension, and a 20 cm 12% sodium dodecyl sulfate gel in the second dimension. From 460 spots consistently detected in 4 separate gels, 80 spots occurred in increased amounts (>1.6 fold) in active fractions versus the inactive flowthrough. We selected 40 spots of these 80 (>3 fold increase) and picked them with an Ettan spot picker (GE Healthcare). Spots were in-gel digested with trypsin, separated on an Ultimate 3000 nano-HPLC (Dionex, Vienna, Austria) and analyzed by nanospray mass spectrometry using an LTQ Orbitrap XL, Thermo Finnigan (Thermo Electron, Vienna, Austria) as described (5). For evaluation of the raw data, the Mascot program (Matrix Sciences, London, UK) and the rat section of the protein database (NCBI, 33,664 sequences) were used. Family with sequence similarity 43, member A (FAM43A) and transmembrane protein 79 (TMEM79) were manually selected for plausibility of a membrane protein with unknown function.

### Determination of Alkylglycerol Monooxygenase Activity.

The assay was performed as described (22). A pyrene-labelled alkylglycerol (1-O-pyrenedecyl-sn-glycerol) was used as substrate which was converted to pyrenedecanal by alkylglycerol monooxygenase. Since pyrenedecanal is stable to aerobic oxidation (31) and cannot not be sufficiently separated from the 1-O-pyreneglycerol substrate by our HPLC system, the assay requires the presence of fatty aldehyde dehydrogenase in the sample which converts pyrenedecanal to pyrenedecanoic acid, the product finally detected by HPLC. Fatty aldehyde dehydrogenase is abundant in mouse tissues (31), and is also present in CHO cells (18.8±1.3 pmol mg⁻¹ min⁻¹, mean±SEM, n=4). In some recombinant expression experiments, fatty aldehyde dehydrogenase in CHO cells was increased by recombinant overexpression, or supplied to Xenopus laevis oocytes by cRNA injection. For protein purification experiments, 14 pmol ml⁻¹ min⁻¹ recombinant rat fatty aldehyde dehydrogenase (31) was added to the reaction mixture. 10 μl alkylglycerol monooxygenase reaction mixture contained 100 mM Tris HCl pH 8.5, 0.1 mg/ml catalase, 0.2 mM NAD, 0.2 mM NADPH (all from Sigma), 0.1 mM 1-O-pyrenedecyl-sn-glycerol (chemically synthesized from pyrenedecanoic acid and glycerol as described (22)), 0.2 μg/ml (0.5 μmol ml⁻¹ min⁻¹) recombinant Physarum polycephalum dihydropteridine reductase (36) and 0.2 mM tetrahydrobiopterin (Schircks, Jona, Switzerland). The reaction was started by addition of the protein and incubated for 60 min at 37° C. in the dark. Negative controls without protein (concentration of pyrenedecanoic acid <1 nM) and rat liver microsomes as positive controls were always run in parallel. After addition of 30 μl methanol and centrifugation for 5 min at 16,000 g, 10 μl of the sample were injected to a Zorbax XDB-C8 rapid resolution column (Agilent Technologies, Vienna, Austria) using an Agilent 1200 Series HPLC system. Elution (flow rate 1.0 ml/min) was performed with a mixture of 21% (v/v) 10 mM potassium phosphate buffer, pH 6.0 and 79% (v/v) methanol for 4.5 min, followed by a gradient to 100% methanol at 5.0 min. At 8.0 min, the initial buffer/methanol (21:79) mix was re-established and the column equilibrated until 8.5 min. Pyrenedecanoic acid was detected by fluorescence (340 nm excitation and 400 nm emission, detection limit 1 nM).

### Determination of Fatty Aldehyde Dehydrogenase Activity.

The assay was performed as described (31). 10 μl reaction mixture contained 20 mM sodium pyrophosphate, pH 8.0, 1 mM NAD, 1% (v/v) Triton X-100 (reduced form, Fluka, Buchs, Switzerland) and 50 μM of the substrate pyrenedecanal (Ramidus AB, Lund, Sweden) which was added from a 40× stock solution in ethanol. The reaction was started by addition of protein sample. After incubation for 10 minutes at 37° C. in the dark, the reaction was stopped with 30 μl methanol. For HPLC analysis the same Agilent system and column were used as for the alkylglycerol monooxygenase assay. The elution buffer consisted of a mixture of 18.75% (v/v) 10 mM potassium phosphate buffer, pH 6.0 and 81.25% (v/v) methanol. Samples were eluted at a flow rate of 1.0 ml/min for 8.0 min, followed by a gradient to 100% methanol at 8.5 min. After a run time of 12.5 min, the original buffer/methanol composition (18.75:81.25) was restored within 30 seconds, resulting in a total run time of 13.0 min. Pyrenedecanoic acid was detected by fluorescence (340 nm excitation and 400 nm emission, detection limit 1 nM).

### Proteomic Analysis of Partially Purified Alkylglycerol Monooxygenase Fractions.

After solubilization of rat liver microsomes with 0.5% (w/v) cholate and 20% (v/v) glycerol, alkylglycerol monooxygenase was purified over ω-aminohexyl sepharose and hydroxylapatite columns. Proteins differing in the inactive flowthrough and the active fractions eluting from the hydroxylapatite column were determined by two dimensional gel electrophoresis using combined separation of proteins labelled with different fluorescent dyes, and monitoring of protein concentration differences by three color fluorescence imaging (DIGE system, GE Healthcare, Vienna Austria). Spots were collected with a spot picker, digested with trypsin in the gel pieces, separated by nano HPLC and analyzed by electron spray ionization mass spectrometry as described (37).

### Screening of Pools of a Rat Liver Expression Library.

A Superscript rat liver expression library (Invitrogen, Carlsbad, Calif., USA) was divided to 196 pools containing about 2,500 independent clones, the DNA of the pools was transfected to CHO cells, the cells harvested after 24 hours and alkylglycerol monooxygenase activity measured in protein extracts.

Preparation of Plasmid Pools from a Rat Liver cDNA Expression Library.

0.1 μl (approximately 500,000 colony forming units) of a Superscript rat liver cDNA expression library (10654-010, Invitrogen, Carlsbad, Calif., USA) were diluted to 1.5 ml in Luria Bertani Broth (Difco, Detroit, Mich., USA) containing 50 μg/ml (w/v) ampicillin (Serva, Heidelberg, Germany) and spread on a 22×22 cm agar plate containing 50 μg/ml (w/v) ampicillin, incubated over night at 30° C. and cut into 196 individual pieces of equal size. From each of these pieces, bacteria were grown over night at 30° C. in Terrific Broth (Roth, Karlsruhe, Germany) containing 50 μg/ml (w/v) ampicillin. Bacteria were collected by centrifugation, and plasmids prepared with a Jetstar 2.0 Mini Kit (Genomed, Lohne, Germany) using a low endotoxin protocol according to the manufacturers instructions.

Transfection of CHO Cells with Candidate Genes.

Expression plasmids of candidate genes were obtained from Imagenes (Berlin, Germany) or Origene (Rockville Md. USA), except for ALDH3A2 which was cloned from a rat liver cDNA to a pcDNA3.1+ expression vector (Invitrogen) by standard techniques. Plasmids were transfected to CHO cells using ExGen 500 (Fermentas, St. Leon-Rot, Germany), the cells cultivated for 24 hours, harvested and alkylglycerol monooxygenase and fatty aldehyde dehydrogenase activities determined.

### Transfection of CHO Cells.

80,000 Chinese hamster ovary Ki cells (CHO-K1, LGC Promochem, Wesel, Germany) were transfected with 1 μg of expression plasmids or plasmid pools using the ExGen 500 transfection agent (Fermentas, St. Leon-Rot, Germany) and standard protocols. Cells were cultivated for 24 h in 1 ml F12-K medium containing 10% (v/v) heat-inactivated fetal calf serum and 1 μM of the tetrahydrobiopterin precursor sepiapterin (Schircks, Jona, Switzerland). For control of transfection efficiency, a plasmid encoding enhanced green fluorescent protein (pEGFP-N1, Clonetech, Mountain View, Calif., USA) was used and fluorescence quantified directly in the culture plates by a Typhoon 9410 scanner (GE Healthcare). Cells were harvested by trypsinization, washed and pellets collected. Pellets were opened in distilled water containing 0.5% (w/v) CHAPS and a protease inhibitor mix (GE Healthcare). Alkylglycerol monooxygenase activity (22) and fatty aldehyde dehydrogenase activity (31) were then determined as described. Human and murine expression plasmids were obtained from Imagenes (Berlin, Germany) or Origene (Rockville, Md., USA, obtained via VWR, Vienna, Austria): C17orf28, human, Imagenes IRATp970F1147D, IMAGE 5174235; CCDCl32, human, Origene SC107550, NM_{—}024553.2; C11orf2, human, Origene SC319162, NM_{—}013265.2; Moxdl, mouse, Origene MC203248, NM_{—}021509; TMTC2, human, Origene SC127237, NM_{—}152588.1; Sc4 mol, mouse, Origene MC203758, NM_{—}025436; C5orf4, human, Imagenes IRAUp969E0937D, IMAGE2906244; TMEM195, human, Imagenes IRATp970A09112D, IMAGE 6152531; FAM43A, human, Origene SC100512, NM_{—}153690.4; Tmem79, mouse, Origene MC200024, NM_{—}024246. An ALDH3A2 expression plasmid to deliver fatty aldehyde dehydrogenase activity was generated from rat liver cDNA by polymerase chain reaction and subcloning to pcDNA3.1+ (Invitrogen) using standard protocols. The insert sequence was confirmed to match the reading frame of NM_{—}031731.2.

Injection of Alkylglycerol Monooxygenase and Fatty Aldehyde Dehydrogenase cRNAs into Xenopus laevis Oocytes.

Stage V-VI oocytes harvested from Xenopus laevis (kindly supplied by Igor Baburin and Steffen Hering, University of Vienna) were incubated for 45-60 min in collagenase type I (1 mg/ml, Sigma). After extensive washes in OR2 solution (5 mM HEPES pH 7.5 containing 82.5 mM NaCl, 2 mM KCl and 1 mM MgCl₂), the follicle layer was removed mechanically. Oocytes were injected after an overnight rest in ND96 solution (5 mM HEPES pH 7.5 containing 96 mM NaCl, 2 mM KCl, 1 mM MgCl₂, 1.8 mM CaCl₂, 37,650 U/ml penicillin and 50,700 U/ml streptomycin) with polyadenylated capped cRNA (27-69 ng in diethylpyrocarbonate (DEPC, Sigma) treated water) of TMEM195, ALDH3A2 and 1:1 mixes of the two cRNAs. 3-4 days after injection, two oocytes per condition were harvested and homogenized together in 200 μl 100 mM Tris HCl pH 7.6 containing 250 mM sucrose and 1 mM phenylmethylsulphonyl fluoride (PMSF, Sigma). Protein concentration was determined by the Bradford assay using bovine serum albumin as standard. Samples were diluted to 1 mg/ml with 100 mM Tris HCl pH 8.5 and analyzed using the alkylglycerol monooxygenase (22) and fatty aldehyde dehydrogenase (31) assay. Oocytes injected in parallel with 50 n1 of DEPC treated water served as controls.

### Preparation of Mouse Tissues and Cells.

C57b1/6 mice (20 g) were obtained from Charles River Laboratory (Sulzfeld, Germany). Mouse tissues (kindly provided by Manuel Maglione, Innsbruck Medical University) were homogenized in 200-500 μl homogenization buffer (0.1 M Tris HCl, 0.25 M sucrose, 1 mM freshly added PMSF, pH 7.6) depending on the size of the tissue using an Ultraturrax mixer (Ika, Stauffen, Germany). Samples were centrifuged for 10 min at 16,000 g at 4° C. and protein concentration was determined in the supernatant by Bradford assay using bovine serum albumin as standard. Supernatants were diluted to 1-2 mg/ml protein with 100 mM Tris HCl at pH 8.5 and the activities of alkylglycerol monooxygenase (22) determined. RAW 267.4 cells were obtained from American Type Culture Collection, Rockville, Md., USA. NIH 3T3 cells were kindly provided by Muhammed Saeed, Innsbruck Medical University. Mouse embryonic fibroblasts (MEF) were kindly provided by Reinhard Sigl, Innsbruck Medical University) cells were opened as described for CHO cells and activity of alkylglycerol monooxygenase was determined (22).

### Selection of Alkylglycerol Monooxygenase Candidate Genes.

The alignment of the tetrahydrobiopterin binding region of aromatic amino acid hydroxylases and nitric oxide synthase presented by (34) was translated to the following protein motif: (R,K)(G,A,L,M,I,V,N)X{1,2}(C,S,T)X{3,4}(A,I,V,L,M)X{4,5}PX{2,3}(S,T)XXPX{2,3}H X{,1}(D,E)(A,M,L,V,I,F)(A,L,M,V,I,F,Y), and Uniprot release 4.6 (1.8 million sequences) searched using the findpatterns program of the gcg program package (version 10.3, Accelrys, San Diego, USA) locally installed on a Silicon Graphics (Fremont, Calif., USA) Origin 2000 server. Proteins were screened for structural features common with phenylalanine hydroxylase using a method for prediction of protein compactness and local structural features (38). PFAM motives were browsed on the public server of the Wellcome Trust Sanger Institute, Hinxton, UK (http://pfam.sanger.ac.uk/).

### Statistics.

Values were compared by one way analysis of variance with Bonferroni correction, p values <0.05 were considered significant. Correlation of TMEM195 mRNA expression levels with alkylglycerol monooxygenase activity was assessed by linear and by Spearman rank correlation analysis. All computations were performed by the Graph Pad Prism Program 3.0 (GraphPad Software Inc., San Diego, Calif., USA). Data are presented as mean±standard error of the mean (SEM) for three independent experiments unless stated differently.

### EXAMPLE II

### Protein Purification and Functional Expression Screening Attempts

Although we used a novel, robust and sensitive assay for alkylglycerol monooxygenase activity (22), all our attempts to purify the protein from male rat liver, the source with the highest activity observed (14), failed due to the instability of the enzyme activity (FIG. 9). Only partial solubilization could be achieved without loss of activity (FIG. 4). The necessity of HPLC analysis which has a capacity of about 40 assays a day precluded a full, single clone functional expression screen which would require testing of about 100,000 clones. We therefore tried an approach using plasmid pools in a way successfully applied to the cloning of an endothelin receptor (39). Transfection of Chinese hamster ovary (CHO) cells with 196 plasmid pools containing about 2,500 individual clones each of a rat liver expression library, however, did not yield activity above background. We then took advantage of the availability of the extensive set of human and murine open reading frames (2) from databases which we browsed for candidates by bioinformatic and proteomic approaches, and selected the ten most promising candidates for transfection experiments.

### EXAMPLE III

### Candidate Genes from Bioinformatic and Proteomic Analyses

We found no significant primary protein sequence homologues of the tetrahydrobiopterin binding region of aromatic amino acid hydroxylases in addition to the already assigned genes phenylalanine hydroxylase (PAH), tyrosine hydroxylase (TH) and the two tryptophan hydroxylases (TPH1 and TPH2) in the databases. A pattern matching the non-significant, forced primary sequence alignment of aromatic amino acid hydroxylases and nitric oxide synthases (34) was observed to be specific for the sequences of all known tetrahydrobiopterin-dependent animal proteins, i.e. aromatic amino acid hydroxylases and nitric oxide synthases. Allowing one mismatch, 48 human proteins were observed to contain this motif. From these we selected chromosome 17 open reading frame 28 (C17orf28). To screen for 3D-structural homologues of phenylalanine hydroxylase, all sequences of the human proteome were subjected to a meta-structure (38) calculation and aligned with phenylalanine hydroxylase. Pairwise meta-structure similarities were quantified based on compactness and second structure values. From 100 best scoring hits we chose three plausible candidates, coiled-coil domain containing 132 (CCDC132), chromosome 11 open reading frame 2 (C11 orf2) and dopamine beta hydroxylase like monooxygenase 1 (MOXD1). Transmembrane and tetratricopeptide repeat containing 2 (TMTC2) attracted our attention due to the occurrence of a conserved domain with unknown function (PFAM08409). PFAM motifs characterize amino acid combinations in primary protein sequences which are characteristic for properties and functions of proteins (35). 11,912 families of proteins had been defined by such motifs in October 2009. When browsing PFAM motifs we realized that the fatty acid hydroxylase motif (PFAM04116) is found in proteins that catalyze hydroxylations of saturated aliphatic carbons in a way similar to alkylglycerol monooxygenase, though no tetrahydrobiopterin dependence of any of these reactions had been described so far. From the human proteins containing the fatty acid hydroxylase motif, we selected three with suspected potential for undiscovered roles, sterol-C4-methyl oxidase-like (SC4MOL), chromosome 5 open reading frame 4 (C5orf4), and transmembrane protein 195 (TMEM195). From proteomic analysis of partially purified fractions of alkylglycerol monooxygenase from rat liver we chose family with sequence similarity 43, member A (FAM43A) and transmembrane protein 79 (TMEM79).

### EXAMPLE IV

### Transfection of Chinese Hamster Ovary Cells with Expression Plasmids of Candidate Genes

The results of transfection of the ten selected expression plasmids of human or murine reading frames in CHO cells are shown in FIG. 5A. TMEM195 transfection led to an increase in tetrahydrobiopterin-dependent alkylglycerol monooxygenase activity (p<0.001), all other plasmids or plasmid-free controls yielded baseline activities independent of tetrahydrobiopterin. Cotransfection of TMEM195 with ALDH3A2 (fatty aldehyde dehydrogenase) yielded a two orders of magnitude higher readout in the alkylglycerol monooxygenase assay as compared to transfection of TMEM195 alone (p<0.001, FIG. 5B), reaching levels one order of magnitude higher than those observed in mouse tissues (FIG. 6A). Thus, fatty aldehyde dehydrogenase activity present in CHO cells (FIG. 7A) limited the amount of recombinant alkylglycerol monooxygenase activity detected with our coupled assay (22). This is consistent with the notion that an aldehyde (13) is the product of the TMEM195 encoded alkylglycerol monooxygenase activity (FIG. 3).

Alkylglycerol monooxygenase activity generated in CHO cells by transfection of TMEM195 and ALDH3A2 displayed a Michaelis Menten constant (K_{M}) of 11.0±1.1 μM for 1-O-pyrenedecylglycerol and 2.58±0.42 μM for tetrahydrobiopterin. These biochemical parameters are almost identical to those found in rat liver microsomes (K_{M} 8.90 μM for 1-O-pyrenedecylglycerol, 2.60 μM for tetrahydrobiopterin (22)). 1,10-Phenanthroline, an iron chelator, inhibited alkylglycerol monooxygenase activity generated in CHO cells by transfection in micromolar concentrations (50% inhibition at 1.39±0.38 μM) in a manner similar to observations with rat liver microsomes (28).

### EXAMPLE V

### Injection of TMEM195 and ALDH3A2 cRNAs to Xenopus Laevis Oocytes

To confirm alkylglycerol monooxygenase sequence assignment to TMEM195 in an independent system, we injected polyadenylated and capped TMEM195 and/or ALDH3A2 cRNA or water into Xenopus laevis oocytes, harvested them after 3-4 days and analyzed them for alkylglycerol monooxygenase (22) and fatty aldehyde dehydrogenase (31) activities.

We identified tetrahydrobiopterin-dependent alkylglycerol monooxygenase activity in oocytes injected with TMEM195 (p<0.05) which was stimulated about twofold (p<0.001) by coinjection with ALDH3A2 (FIG. 5C). Like for alkylglycerol monooxygenase activities (FIG. 5B, FIG. 5C), fatty aldehyde dehydrogenase activities reached by injection of ALDH3A2 cRNA into oocytes (FIG. 7B) were two orders of magnitude lower than those achieved in CHO cells by transfection with an expression plasmid (FIG. 7A).

### EXAMPLE VI

### Comparison of Alkylglycerol Monooxygenase Activities and Occurrence of TMEM195 mRNA

TMEM195 mRNA levels in mouse tissues and cells available from public databases correlated significantly with alkylglycerol monooxygenase activities measured (FIG. 6B). In addition to human cells, mouse and rat tissues, we detected tetrahydrobiopterin-dependent alkylglycerol monooxygenase activity in chicken liver (strain HB15-FINN, 52.4±1.5 pmol mg⁻¹ min⁻¹), zebrafish liver (strain Tubingen longfin, 27.1±5.3 pmol mg⁻¹ min⁻¹) but not in Drosophila melanogaster (strain Oregon R), Aspergillus fumigatus (strain ATCC46645), Aspergillus nidulans (strain A89), Physarum polycephalum (strain CS310), Saccharomyces cerevisiae (strain Y187) or Escherichia coli (strain BL21DE3) where all activities were below 1 pmol mg⁻¹ min⁻¹. This pattern is consistent with the occurrence of TMEM195 related sequences in the NCBI databases, which characterize it to be a gene conserved in Bilateria (Homologene 45620).

### EXAMPLE VII

### Sequence Assignment of Alkylglycerol Monooxygenase Activity to Transmembrane Protein 195

Like other researchers in the last 45 years, we were not able to purify alkylglycerol monooxygenase although we used a novel, robust and sensitive assay for this enzyme activity (22). We could also not assign the sequence by transfection of pools of expression plasmids and rounds of selection until a single clone was obtained, a method that had been successfully used by other researchers to clone genes that could be assayed by a sensitive functional assay, but had not been accessible by protein purification (39). The assignment of the transmembrane protein 195 sequence to alkylglycerol monooxygenase activity presented here is therefore based on the induction of enzymatic activity by transfection of expression plasmids of sequences selected from bioinformatic searches or proteomic analysis of partially purified fractions of the enzyme. In CHO cells, an expression plasmid for transmembrane protein 195 induced a tetrahydrobiopterin-dependent alkylglycerol monooxygenase activity that was tenfold higher than in any mouse tissue observed if sufficient fatty aldehyde dehydrogenase activity was supplied by coexpression. The biochemical properties of this induced activity closely resembled the enzyme activity observed in rat liver microsomes in terms of K_{M} values for substrate and cofactor, and sensitivity to inhibition by the iron chelator 1,10-phenanthroline. This sequence assignment was confirmed by generation of alkylglycerol monooxygenase activity by injection of transmembrane protein 195 cRNA into Xenopus oocytes. In addition, the occurrence of the transmembrane protein 195 and alkylglycerol monooxygenase activity among species (the bilateral animals) is consistent with this assignment. In mouse tissues and cells, alkylglycerol monooxygenase activity correlates well with the amount of transmembrane protein 195 mRNA observed. To the best of our knowledge, this report is the first to characterize the sequence of alkylglycerol monooxygenase, the fifth known tetrahydrobiopterin-dependent enzymatic reaction (FIG. 1A).

### EXAMPLE VIII

### Transmembrane Protein 195 is a Tetrahydrobiopterin-Dependent Fatty Acid Hydroxylase Type Enzyme

In addition to defining a functional role for transmembrane protein 195, another novel finding of our work is the requirement of tetrahydrobiopterin for a fatty acid hydroxylase motif containing enzyme. While the PFAM04116 fatty acid hydroxylase motif defines an abstract amino acid matrix, previous work has described this motif as a pattern of eight conserved histidines (40), which is also found in transmembrane protein 195 (FIG. 8). Some of these histidines are required to bind the iron atoms constituting the diiron center which is essential for catalysis (41). This corresponds well to our observation that alkylglycerol monooxygenase activity can be inhibited by the iron chelator 1,10-phenanthroline (28).

No fatty acid hydroxylase motif containing membrane protein has been reported to be purified to homogeneity from a mammalian source so far. Presumably for all members of this family purification attempts faced similar problems of low stability as we and others experienced with alkylglycerol monooxygenase. Most of the respective human genes, i.e. fatty acid 2-hydroxylase (42), sterol-C5-desaturase (43) and sterol-C4-methyl oxidase-like (44), have been assigned by homology to a characterized yeast gene of similar function. Cholesterol 25-hydroxylase, in contrast, has been cloned by transfection of cDNA pools (45). The radiometric assay for cholesterol 25-hydroxylase used in (45) was sensitive enough to detect the activity upon transfection of pools of 3,000-4,000 clones, whereas we detected only background using pools of 2500 clones each using our fluorescence-HPLC assay for alkylglycerol monooxygenase (22).

It will be fascinating to learn why transmembrane protein 195 essentially requires the additional cofactor tetrahydrobiopterin for cleavage of the O-alkyl ether bond which the other fatty acid hydroxylase motif containing proteins apparently do not need. On the other hand, all characterized diiron hydroxylases include a multisubunit hydroxylase, electron transfer proteins and a cofactorless effector protein that is unique to the diiron hydroxylase family (46). In transmembrane protein 195, tetrahydrobiopterin might substitute for one or more of these additional protein components.

### EXAMPLE IX

### Sequence Homology Analysis Suggests that Alkylglycerol Monooxygenase Forms a Distinct Third Group Among Tetrahydrobiopterin-Dependent Enzymes

After characterization of aromatic amino acid hydroxylases, the first known tetrahydrobiopterin-dependent enzymes, the sequence of a novel class of tetrahydrobiopterin-dependent enzymes, the nitric oxide synthases, was described about two decades ago (47). Biochemical research has subsequently outlined how nitric oxide synthases differ from cytochrome P450 monooxygenases, making them dependent on the additional cofactor tetrahydrobiopterin and enabling them to avoid formation of an autoinhibitory heme-NO complex (9). Sequence homology suggests that transmembrane protein 195 is equipped with a diiron center for hydroxylation of aliphatic hydrocarbons. This distinguishes alkylglycerol monooxygenase biochemically from the other two known classes of tetrahydrobiopterin-dependent enzymes, which contain a heme iron like the nitric oxide synthases or a single, non-heme iron like aromatic amino acid hydroxylases. Similar to this classification by the form of iron contained in the enzymes, primary amino acid sequence comparison clustered tetrahydrobiopterin-dependent enzymes to three independent groups, the aromatic amino acid hydroxylases, the alkylglycerol monooxygenase and the nitric oxide synthases. These clusters show no primary sequence homology across groups (FIG. 1B).

### EXAMPLE X

### The Sequence Assignment Will Facilitate the Study of the Physiological Role of Alkylglycerol Monooxygenase

1-O-Alkylglycerol-derived lipids can modulate signal transduction (26) and are required for nerve and sperm development as well as for protection of the eye from cataract, constitute a component of the glycosylphosphatidylinositol (GPI) anchor, or, as in the case of platelet activating factor, are mediators themselves (23). Decreased concentrations of ether lipids have been reported to be associated with hypertension (48). The assignment of the sequence of alkylglycerol monooxygenase to transmembrane protein 195 will enable research on the physiological significance of this enzyme, which degrades these lipids and may contribute to regulation of their in vivo concentration.

### LITERATURE REFERENCES

1. Ota, T., et al. (2004) Complete sequencing and characterization of 21,243 full-length human cDNAs. Nat. Genet. 36, 40-45
2. Maeda, N., et al. (2006) Transcript annotation in FANTOM3: mouse gene catalog based on physical cDNAs. PLoS. Genet. 2, e62
3. Lespinet, O., Labedan, B. (2005) Orphan enzymes? Science 307, 42
4. Lespinet, O., Labedan, B. (2006) Puzzling over orphan enzymes. Cell Mol. Life Sci. 63, 517-523
5. Werner-Felmayer, G et al. (2002) Tetrahydrobiopterin biosynthesis, utilizationand pharmacological effects. Curr Drug Metab 3:159-173.
6. Thony, B et al. (2000) Tetrahydrobiopterin biosynthesis, regeneration and functions. Biochem J 347 Pt 1:1-16.
7. Stuehr, D J (1999) Mammalian nitric oxide synthases. Biochim Biophys Acta 1411:217-230.
8. Marietta, M A et al. (1998) Catalysis by nitric oxide synthase. Curr Opin Chem Biol 2:656-663.
9. Gorren, A C Mayer, B (2007) Nitric-oxide synthase: a cytochrome P450 family foster child. Biochim Biophys Acta 1770:432-445.
10. Palmer, R M et al. (1987) Nitric oxide release accounts for the biological activity of endothelium-derived relaxing factor. Nature 327:524-526.
11. Bogdan, C (2001) Nitric oxide and the immune response. Nat Immunol 2:907-916.
12. Mustafa, A K et al. (2009) Signaling by gasotransmitters. Sci Signal 2:re2.
13. Tietz, A et al. (1964) A new pteridine-requiring enzyme system for the oxidation of glyceryl ethers. J Biol Chem 239:4081-4090.
14. Taguchi, H Armarego, W L (1998) Glyceryl-ether monooxygenase [EC 1.14.16.5]. A microsomal enzyme of ether lipid metabolism. Med Res Rev 18:43-89.
15. Lespinet, O Labedan, B (2006) ORENZA: a web resource for studying ORphan ENZyme activities. BMC Bioinformatics 7:436.
16. Citron, B A et al. (1993) Mutation in the 4a-carbinolamine dehydratase gene leads to mild hyperphenylalaninemia with defective cofactor metabolism. Am J Hum Genet. 53:768-774.
17. Soodsma, J. F., et al. (1972) Partial characterization of the alkylglycerol cleavage enzyme system of rat liver. J. Biol. Chem. 247, 3923-3929
18. Ishibashi, T., Imai, Y. (1983) Solubilization and partial characterization of alkylglycerol monooxygenase from rat liver microsomes. Eur. J. Biochem. 132, 23-27
19. Koetting, J., et al. (1987) A continuous assay for O-alkylglycerol monooxygenase (E.C. 1.14.16.5). Lipids 22, 824-830
20. Kaufman, S., et al. (1990) Dependence of an alkyl glycol-ether monooxygenase activity upon tetrahydropterins. Biochim. Biophys. Acta 1040, 19-27
21. Taguchi, H., et al. (1994) Glyceryl-ether monooxygenase (EC 1.14.16.5): nature of the glyceryl-ether lipid substrates in aqueous buffer. Biol. Chem. Hoppe Seyler 375, 329-334
22. Werner, E. R., et al. (2007) Widespread occurrence of glyceryl ether monooxygenase activity in rat tissues detected by a novel assay. J. Lipid Res. 48, 1422-1427
23. Gorgas, K., et al. (2006) The ether lipid-deficient mouse: tracking down plasmalogen functions. Biochim. Biophys. Acta 1763, 1511-1526
24, Teigler, A., et al. (2009) Defects in myelination, paranode organization and Purkinje cell innervation in the ether lipid-deficient mouse cerebellum. Hum. Mol. Genet. 18, 1897-1908
25. Komljenovic, D., et al. (2009) Disruption of blood-testis barrier dynamics in ether-lipid-deficient mice. Cell Tissue Res. 337, 281-299
26. Mandal, A., et al. (1997) Interleukin-1-induced ether-linked diglycerides inhibit calcium-insensitive protein kinase C isotypes. Implications for growth senescence. J. Biol. Chem. 272, 20306-20311
27. Vink, S. R., et al. (2007) Rationale and clinical application of alkylphospholipid analogues in combination with radiotherapy. Cancer Treat. Rev. 33, 191-202
28. Watschinger, K., et al. (2009) Glyceryl ether monooxygenase resembles aromatic amino acid hydroxylases in metal ion and tetrahydrobiopterin dependence. Biol. Chem. 390, 3-10
29. Werner-Felmayer, G., et al. (1994) Pteridine biosynthesis and nitric oxide synthase in Physarum polycephalum. Biochem. J. 304, 105-11130.
30. Golderer, G., et al. (2001) Nitric oxide synthase is induced in sporulation of Physarum polycephalum. Genes Dev. 15, 1299-1309
31. Keller, M A et al. (2009) Monitoring of fatty aldehyde dehydrogenase by formation of pyrenedecanoic acid from pyrenedecanal. J Lipid Res Doi:10.1194/jlr002220.
32. Tamura, K et al. (2007) MEGA4: Molecular Evolutionary Genetics Analysis (MEGA) software version 4.0. Mol Biol Evol 24:1596-1599.
33. Shanklin, J Cahoon, E B (1998) Desaturation and related modifications of fatty acidsl. Annu Rev Plant Physiol Plant Mol Biol 49:611-641.
34. Cho, H. J., et al. (1995) Inducible nitric oxide synthase: identification of amino acid residues essential for dimerization and binding of tetrahydrobiopterin. Proc. Natl. Acad. Sci. U.S.A 92, 11514-11518
35. Finn, R. D., et al. (2008) The Pfam protein families database. Nucleic Acids Res. 36, D281-D288
36. Wild, C et al. (2003) Physarum polycephalum expresses a dihydropteridine reductase with selectivity for pterin substrates with a 6(1′2′-dihydroxypropyl) substitution. Biol Chem 384:1057-1062.
37. Sobieszek, A et al. (2006) Phosphorylation of myorod (catchin) by kinases tightly associated to molluscan and vertebrate smooth muscle myosins. Arch Biochem Biophys 454:197-205.
38. Konrat, R (2009) The protein meta-structure: a novel concept for chemical and molecular biology. Cell Mol Life Sci 66:3625-3639.
39. Elshourbagy, N A et al. (1992) Molecular cloning and characterization of the major endothelin receptor subtype in porcine cerebellum. Mol Pharmacol 41:465-473.
40. Shanklin, J et al. (1994) Eight histidine residues are catalytically essential in a membrane-associated iron enzyme, stearoyl-CoA desaturase, and are conserved in alkane hydroxylase and xylene monooxygenase. Biochemistry 33:12787-12794.
41. Shanklin, J et al. (2009) Desaturases: emerging models for understanding functional diversification of diiron-containing enzymes. J Biol Chem 284:18559-18563.
42. Alderson, N L et al. (2004) The human FA2H gene encodes a fatty acid 2-hydroxylase. J Biol Chem 279:48562-48568.
43. Matsushima, M et al. (1996) Molecular cloning and mapping of a human cDNA (SCSDL) encoding a protein homologous to fungal sterol-C5-desaturase. Cytogenet Cell Genet. 74:252-254.
44. Li, L Kaplan, J (1996) Characterization of yeast methyl sterol oxidase (ERG25) and identification of a human homologue. J Biol Chem 271:16927-16933.
45. Lund, E G et al. (1998) cDNA cloning of mouse and human cholesterol 25-hydroxylases, polytopic membrane proteins that synthesize a potent oxysterol regulator of lipid metabolism. J Biol Chem 273:34316-34327.
46. Leahy, J G et al. (2003) Evolution of the soluble diiron monooxygenases. FEMS Microbiol Rev 27:449-479.
47. Bredt, D S et al. (1991) Cloned and expressed nitric oxide synthase structurally resembles cytochrome P-450 reductase. Nature 351:714-718.
48. Graessler, J et al. (2009) Top-down lipidomics reveals ether lipid deficiency in blood plasma of hypertensive patients. PLoS One 4:e6261.

### SEQUENCES

The present invention refers to the following nucleotide and amino acid sequences:

The sequences provided herein are available in the NCBI database and can be retrieved from http://www.ncbi.nlm.nih.gov/sites/entrez?db=gene; These sequences also relate to annotated and modified sequences. The present invention provides techniques and methods wherein homologous sequences and variants of the concise sequences provided herein are used. Preferably, such “variants” are genetic variants.

SEQ ID NO: 1:
Nucleotide sequence encoding Homo sapiens wild type human alkylglycerol
monooxygenase TMEM195 (coding region).
ATGAAGAACCCAGAAGCCCAGCAGGATGTTTCAGTTTCCCAGGGATTTCGCATGTTGTTTTACACGATGA
AACCCAGTGAAACTTCATTCCAAACATTAGAAGAGGTGCCTGATTATGTAAAAAAGGCAACTCCATTTTT
CATTTCTTTGATGCTGCTTGAACTTGTTGTCAGCTGGATTCTCAAAGGAAAGCCACCAGGTCGCCTGGAT
GATGCTTTAACGTCAATCTCAGCTGGTGTTCTGTCTCGACTTCCAAGTCTATTTTTCAGGAGCATTGAAC
TGACCAGTTATATTTATATCTGGGAGAACTACAGGCTGTTCAATTTGCCTTGGGATTCTCCATGGACTTG
GTATTCAGCCTTCTTAGGAGTTGACTTTGGCTACTACTGGTTCCATCGTATGGCTCATGAAGTTAATATT
ATGTGGGCCGGACACCAAACACATCATAGTTCTGAAGACTATAACTTATCCACAGCACTGAGACAGTCTG
TCCTCCAGATATATACTTCCTGGATTTTCTACTCTCCCCTGGCCCTCTTCATACCCCCTTCAGTATATGC
TGTTCATCTTCAATTCAATCTTCTTTACCAATTTTGGATCCATACAGAGGTCATCAATAACCTTGGTCCT
TTGGAACTGATTCTTAATACTCCTAGCCATCATAGGGTTCATCATGGCAGAAATCGTTATTGCATAGACA
AAAATTATGCTGGTGTTCTTATTATTTGGGATAAAATTTTTGGGACATTTGAAGCAGAAAATGAAAAAGT
TGTATATGGCTTAACACATCCCATTAATACATTTGAACCAATCAAAGTGCAGTTCCATCACTTATTTTCC
ATATGGACTACATTCTGGGCCACACCTGGATTCTTCAATAAGTTTTCTGTCATATTTAAGGGACCGGGAT
GGGGTCCAGGTAAACCAAGACTTGGTCTCAGTGAAGAAATTCCAGAGGTCACCGGCAAAGAAGTTCCCTT
CTCATCATCTTCATCTCAGCTATTAAAGATATATACAGTTGTACAGTTTGCTCTGATGTTGGCATTTTAT
GAAGAGACCTTTGCAGATACAGCTGCACTGTCGCAAGTTACTCTCCTTCTGAGGGTTTGCTTCATTATCC
TGACCTTGACTTCCATTGGATTTCTTCTGGATCAAAGACCCAAGGCAGCTATTATGGAAACTCTCCGTTG
CTTGATGTTCTTAATGCTGTACCGATTTGGTCACCTGAAGCCTCTTGTCCCTTCATTGTCATCTGCTTTT
GAGATTGTTTTTTCCATTTGCATTGCTTTCTGGGGAGTTAGAAGCATGAAACAACTCACCTCTCACCCTT
GGAAATAA
SEQ ID NO: 2:
Amino acid sequence of wild type human alkylglycerol monooxygenase TMEM195.
MKNPEAQQDVSVSQGFRMLFYTMKPSETSFQTLEEVPDYVKKATPFFISLMLLELVVSWILKGKPPGRLD
DALTSISAGVLSRLPSLFFRSIELTSYIYIWENYRLFNLPWDSPWTWYSAFLGVDFGYYWFHRMAHEVNI
MWAGHQTHHSSEDYNLSTALRQSVLQIYTSWIFYSPLALFIPPSVYAVHLQFNLLYQFWIHTEVINNLGP
LELILNTPSHHRVHHGRNRYCIDKNYAGVLIIWDKIFGTFEAENEKVVYGLTHPINTFEPIKVQFHHLFS
IWTTFWATPGFFNKFSVIFKGPGWGPGKPRLGLSEEIPEVTGKEVPFSSSSSQLLKIYTVVQFALMLAFY
EETFADTAALSQVTLLLRVCFIILTLTSIGFLLDQRPKAAIMETLRCLMFLMLYRFGHLKPLVPSLSSAF
EIVFSICIAFWGVRSMKQLTSHPWK
SEQ ID NO: 3:
Human alkylglycerol monooxygenase TMEM195.
CTCTCTACACAGAATCGGCTGTTGAGTGTGCTCTCAGTGGAGCTTTGGTTTTAGCTGTTCTCTGACAAAG
AGCTTGTTCTGAGCTGCACATCTCGTCCTCTTTGTTCAGCCTCAGGCTTCAAGCATTGAATCCTAAATAT
TCTCCAGCTGGGAATCAGACAAGGGCAGAAATGAAGAACCCAGAAGCCCAGCAGGATGTTTCAGTTTCCC
AGGGATTTCGCATGTTGTTTTACACGATGAAACCCAGTGAAACTTCATTCCAAACATTAGAAGAGGTGCC
TGATTATGTAAAAAAGGCAACTCCATTTTTCATTTCTTTGATGCTGCTTGAACTTGTTGTCAGCTGGATT
CTCAAAGGAAAGCCACCAGGTCGCCTGGATGATGCTTTAACGTCAATCTCAGCTGGTGTTCTGTCTCGAC
TTCCAAGTCTATTTTTCAGGAGCATTGAACTGACCAGTTATATTTATATCTGGGAGAACTACAGGCTGTT
CAATTTGCCTTGGGATTCTCCATGGACTTGGTATTCAGCCTTCTTAGGAGTTGACTTTGGCTACTACTGG
TTCCATCGTATGGCTCATGAAGTTAATATTATGTGGGCCGGACACCAAACACATCATAGTTCTGAAGACT
ATAACTTATCCACAGCACTGAGACAGTCTGTCCTCCAGATATATACTTCCTGGATTTTCTACTCTCCCCT
GGCCCTCTTCATACCCCCTTCAGTATATGCTGTTCATCTTCAATTCAATCTTCTTTACCAATTTTGGATC
CATACAGAGGTCATCAATAACCTTGGTCCTTTGGAACTGATTCTTAATACTCCTAGCCATCATAGGGTTC
ATCATGGCAGAAATCGTTATTGCATAGACAAAAATTATGCTGGTGTTCTTATTATTTGGGATAAAATTTT
TGGGACATTTGAAGCAGAAAATGAAAAAGTTGTATATGGCTTAACACATCCCATTAATACATTTGAACCA
ATCAAAGTGCAGTTCCATCACTTATTTTCCATATGGACTACATTCTGGGCCACACCTGGATTCTTCAATA
AGTTTTCTGTCATATTTAAGGGACCGGGATGGGGTCCAGGTAAACCAAGACTTGGTCTCAGTGAAGAAAT
TCCAGAGGTCACCGGCAAAGAAGTTCCCTTCTCATCATCTTCATCTCAGCTATTAAAGATATATACAGTT
GTACAGTTTGCTCTGATGTTGGCATTTTATGAAGAGACCTTTGCAGATACAGCTGCACTGTCGCAAGTTA
CTCTCCTTCTGAGGGTTTGCTTCATTATCCTGACCTTGACTTCCATTGGATTTCTTCTGGATCAAAGACC
CAAGGCAGCTATTATGGAAACTCTCCGTTGCTTGATGTTCTTAATGCTGTACCGATTTGGTCACCTGAAG
CCTCTTGTCCCTTCATTGTCATCTGCTTTTGAGATTGTTTTTTCCATTTGCATTGCTTTCTGGGGAGTTA
GAAGCATGAAACAACTCACCTCTCACCCTTGGAAATAACCTGAATTTGTACATAATTCTCTTCTTTTAAT
GAGTTGTCCACACGCATATTATGACTGCATATTAAAATGTAATTATTTTATGTAATGCTTATATGAACTA
TTTCTTCAATGAAAAGTAAAATTACTTATTTACTATTGTTTGCCTTTCACATTTGTTATTTTCTATTAAA
AATTAAAGTCAGTTTTGGTTACTTCCCCCCTTTACTACAATTAAAAAAAGATTTCAAATATAATGATGTT
ATATTAACTGATAGCCTTATATGACAAGTATAAAAAGAAGGGATGAAACTTAAAAACAGTAAAAACAAGA
AGGAATATTGCCTTTACATCAATTTGAAAACAATGTTTCCTTTGATGTTTGCTAAAATTATGCATAGATA
CATGTTTGTAGTCATAAAAATGTATTACATTGGTTGTCTTCCTAAGGCCACAGTTACCTTTGCAATCCAT
ATAACCTAAGAAGCTGCATTCCAGAAAAAGACATCACTGAGGCCAGGCGCGGTGGCTCACCCCTGTAATC
CCAGCACTTTGTGGGGCTGAGGTGGGCGGATCATGAGGTCCAGAGATAGAGACCATCCTGGCCAACATGG
TGAAGTCCTGTCTCTACTAAAAATATAAAAATTTAGCTGGACATGGTGGTGTGCGCCTGTAGTCCCAGCT
ACTCTGGAGGCTGAGGCAGGAGAATCGCTTGAACCTGGGAGGCAGAAGTTGGAGTGAGTGGAGATTGCAC
CACTGCACTCCAGCCTGGGCGACAGAGCGAGACTCCGTCTCAAAGAAAAAAGACATCACTGAAAGAAAAA
TGAACAGAATTTGTCAGAATTAGTTTTTTCAACAGGTTACTTTGTCATACATTTCTCTAATATGCTTGGT
CAATTTGTTTTGGCAGACTGGGCAGCATGCAGCAATTCTGCATTATTTAAAGTTATCAGAACAATGTTAA
TTCTCTAAATAAAATTACCCAAGGT

1. A pharmaceutical composition comprising a nucleic acid molecule encoding a alkylglycerol monooxygenase comprising a polynucleotide selected from the group consisting of:
(a) a polynucleotide sequence as shown in SEQ ID NO:1 or a fragment thereof;
(b) a polynucleotide sequence encoding a polypeptide as shown in SEQ ID NO:2 or a fragment thereof;
(c) a polynucleotide sequence which has at least 80% identity to the polynucleotides as defined in (a) or (b) encoding a functional alkylglycerol monooxygenase or a fragment thereof;
(d) a polynucleotide sequence which hybridizes to the polynucleotide sequence of any one of (a) to (c) and whereby the coding strand encodes a functional alkylglycerol monooxygenase or a fragment thereof;
(e) a polynucleotide sequence encoding a polypeptide as encoded by the nucleotide sequence of any one of (a) to (d) wherein at least one amino acid is deleted, substituted, inserted or added and whereby said polynucleotide encodes a alkylglycerol monooxygenase or a fragment thereof;
(f) a polynucleotide sequence being degenerate as a result of the genetic code to the nucleotide sequence as defined in any one of (a) to (e); and
(g) the complementary strand of the polynucleotide of any one of (a) to (f).

2. The pharmaceutical composition of claim 1, wherein the nucleic acid molecule is comprised in a vector.

3. A recombinant host cell comprising an exogenous nucleic acid molecule encoding an alkylglycerol monooxygenase comprising a polynucleotide selected from the group consisting of:
(a) a polynucleotide sequence as shown in SEQ ID NO:1 or a fragment thereof;
(b) a polynucleotide sequence encoding a polypeptide as shown in SEQ ID NO:2 or a fragment thereof;
(c) a polynucleotide sequence which has at least 80% identity to the polynucleotides as defined in (a) or (b) encoding a functional alkylglycerol monooxygenase or a fragment thereof;
(d) a polynucleotide sequence which hybridizes to the polynucleotide sequence of any one of (a) to (c) and whereby the coding strand encodes a functional alkylglycerol monooxygenase or a fragment thereof;
(e) a polynucleotide sequence encoding a polypeptide as encoded by the nucleotide sequence of any one of (a) to (d) wherein at least one amino acid is deleted, substituted, inserted or added and whereby said polynucleotide encodes a alkylglycerol monooxygenase or a fragment thereof;
(f) a polynucleotide sequence being degenerate as a result of the genetic code to the nucleotide sequence as defined in any one of (a) to (e); and
(g) the complementary strand of the polynucleotide of any one of (a) to (f).

4. A method for producing a polypeptide, comprising culturing the recombinant host cell of claim 3 under such conditions that the polypeptide is expressed, and recovering the polypeptide.

5. A pharmaceutical composition comprising an alkylglycerol monooxygenase encoded by a polynucleotide selected from the group consisting of:
(a) a polynucleotide sequence as shown in SEQ ID NO:1 or a fragment thereof;
(b) a polynucleotide sequence encoding a polypeptide as shown in SEQ ID NO:2 or a fragment thereof;
(c) a polynucleotide sequence which has at least 80% identity to the polynucleotides as defined in (a) or (b) encoding a functional alkylglycerol monooxygenase or a fragment thereof;
(d) a polynucleotide sequence which hybridizes to the polynucleotide sequence of any one of (a) to (c) and whereby the coding strand encodes a functional alkylglycerol monooxygenase or a fragment thereof;
(e) a polynucleotide sequence encoding a polypeptide as encoded by the nucleotide sequence of any one of (a) to (d) wherein at least one amino acid is deleted, substituted, inserted or added and whereby said polynucleotide encodes a alkylglycerol monooxygenase or a fragment thereof;
(f) a polynucleotide sequence being degenerate as a result of the genetic code to the nucleotide sequence as defined in any one of (a) to (e); and
(g) the complementary strand of the polynucleotide of any one of (a) to (f).

6. A pharmaceutical composition comprising an antibody that specifically binds an alkylglycerol monooxygenase encoded by a polynucleotide selected from the group consisting of:
(a) a polynucleotide sequence as shown in SEQ ID NO:1 or a fragment thereof;
(b) a polynucleotide sequence encoding a polypeptide as shown in SEQ ID NO:2 or a fragment thereof;
(c) a polynucleotide sequence which has at least 80% identity to the polynucleotides as defined in (a) or (b) encoding a functional alkylglycerol monooxygenase or a fragment thereof;
(d) a polynucleotide sequence which hybridizes to the polynucleotide sequence of any one of (a) to (c) and whereby the coding strand encodes a functional alkylglycerol monooxygenase or a fragment thereof;
(e) a polynucleotide sequence encoding a polypeptide as encoded by the nucleotide sequence of any one of (a) to (d) wherein at least one amino acid is deleted, substituted, inserted or added and whereby said polynucleotide encodes a alkylglycerol monooxygenase or a fragment thereof;
(f) a polynucleotide sequence being degenerate as a result of the genetic code to the nucleotide sequence as defined in any one of (a) to (e); and
(g) the complementary strand of the polynucleotide of any one of (a) to (f).

7. A method for detecting a polypeptide having the amino acid sequence encoded by the nucleic acid molecule as defined in claim 1, the method comprising obtaining an antibody that specifically binds such an polypeptide, contacting a sample suspected of comprising such a polypeptide and detecting the binding of the antibody thereto to thereby detect the polypeptide.

8.-11. (canceled)

12. A pharmaceutical composition comprising an antagonist or inhibitor of alkylglyerol monooxygenase, the antagonist or inhibitor being defined as an RNAi, siRNA, shRNA, aptamer, antisense, or intramer specifically directed against alkylglycerol monooxygenase.

13. A method for assessing the activity of a candidate molecule suspected of being an antagonist/inhibitor of alkylglycerol monooxygenase comprising the steps of:
(a) contacting a cell, tissue or a non-human animal comprising and expressing alkylglycerol monooxygenase with said candidate molecule;
(b) detecting a decrease in alkylglycerol monooxygenase activity; and
(c) selecting a candidate molecule that decreases alkylglycerol monooxygenase activity;
wherein a decrease of the alkylglycerol monooxygenase activity is indicative for the capacity of the selected molecule to have an antiproliferative effect, to counteract hypertension, to restore male fertility or to ameliorate or prevent cataract.

14. A method for assessing the activity of a candidate molecule suspected of being an agonist/activator of alkylglycerol monooxygenase comprising the steps of:
(a) contacting a cell, tissue or a non-human animal comprising and expressing alkylglycerol monooxygenase with said candidate molecule;
(b) detecting an increase in alkylglycerol monooxygenase activity; and
(c) selecting a candidate molecule that increases alkylglycerol monooxygenase activity;
wherein an increase of the alkylglycerol monooxygenase activity is indicative for the capacity of the selected molecule to induce male infertility, to ameliorate both the neurodegeneration or to ameliorate the recent memory loss associated with Alzheimer's disease.

15. The method of claim 13, wherein the decrease in alkylglycerol monooxygenase activity is detected with polynucleotides capable of hybridizing the alkylglycerol monooxygenase sense molecule or with antibodies that selectively bind alkylglycerol monooxygenase.

16. (canceled)

17. Kit useful for assessing the activity of a candidate molecule suspected of being an agonist/activator of alkylglycerol monooxygenase comprising polynucleotides and/or antibodies capable of detecting the activity of alkylglycerol monooxygenase.

18. A method for purifying a polypeptide having the amino acid sequence encoded by the nucleic acid molecule as defined in claim 1, the method comprising obtaining a sample suspected of comprising such a polypeptide, contacting the sample with an antibody that specifically binds such a polypeptide and purifying the polypeptide therefrom.

19. A method of treating hyperproliferation, to counteract hypertension, to restore male fertility or to ameliorate or prevent cataract in a subject, the method comprising administering to the subject a effective amount of a composition in accordance with claim 6 or claim 12.

20. A method to induce male infertility, to ameliorate neurodegeneration or recent memory loss associated with Alzheimer's disease in a subject, the method comprising administering to the subject an effective amount of a composition in accordance with claim 1 or claim 5.

21. The method of claim 14, wherein the increase in alkylglycerol monooxygenase activity is detected with polynucleotides capable of hybridizing the alkylglycerol monooxygenase sense molecule or with antibodies that selectively bind alkylglycerol monooxygenase.


SEQUENCE LISTING (3 sequences)

SEQ ID NO: 1 - DNA, 1338 bp, Homo sapiens
atgaagaacccagaagcccagcaggatgtttcagtttcccagggatttcgcatgttgttttacacgatgaaacccagtgaaacttcattccaaacattagaagaggtgcctgattatgtaaaaaaggcaactccatttttcatttctttgatgctgcttgaacttgttgtcagctggattctcaaaggaaagccaccaggtcgcctggatgatgctttaacgtcaatctcagctggtgttctgtctcgacttccaagtctatttttcaggagcattgaactgaccagttatatttatatctgggagaactacaggctgttcaatttgccttgggattctccatggacttggtattcagccttcttaggagttgactttggctactactggttccatcgtatggctcatgaagttaatattatgtgggccggacaccaaacacatcatagttctgaagactataacttatccacagcactgagacagtctgtcctccagatatatacttcctggattttctactctcccctggccctcttcatacccccttcagtatatgctgttcatcttcaattcaatcttctttaccaattttggatccatacagaggtcatcaataaccttggtcctttggaactgattcttaatactcctagccatcatagggttcatcatggcagaaatcgttattgcatagacaaaaattatgctggtgttcttattatttgggataaaatttttgggacatttgaagcagaaaatgaaaaagttgtatatggcttaacacatcccattaatacatttgaaccaatcaaagtgcagttccatcacttattttccatatggactacattctgggccacacctggattcttcaataagttttctgtcatatttaagggaccgggatggggtccaggtaaaccaagacttggtctcagtgaagaaattccagaggtcaccggcaaagaagttcccttctcatcatcttcatctcagctattaaagatatatacagttgtacagtttgctctgatgttggcattttatgaagagacctttgcagatacagctgcactgtcgcaagttactctccttctgagggtttgcttcattatcctgaccttgacttccattggatttcttctggatcaaagacccaaggcagctattatggaaactctccgttgcttgatgttcttaatgctgtaccgatttggtcacctgaagcctcttgtcccttcattgtcatctgcttttgagattgttttttccatttgcattgctttctggggagttagaagcatgaaacaactcacctctcacccttggaaataa

SEQ ID NO: 2 - protein, 445 aa, Homo sapiens
MKNPEAQQDVSVSQGFRMLFYTMKPSETSFQTLEEVPDYVKKATPFFISLMLLELVVSWILKGKPPGRLDDALTSISAGVLSRLPSLFFRSIELTSYIYIWENYRLFNLPWDSPWTWYSAFLGVDFGYYWFHRMAHEVNIMWAGHQTHHSSEDYNLSTALRQSVLQIYTSWIFYSPLALFIPPSVYAVHLQFNLLYQFWIHTEVINNLGPLELILNTPSHHRVHHGRNRYCIDKNYAGVLIIWDKIFGTFEAENEKVVYGLTHPINTFEPIKVQFHHLFSIWTTFWATPGFFNKFSVIFKGPGWGPGKPRLGLSEEIPEVTGKEVPFSSSSSQLLKIYTVVQFALMLAFYEETFADTAALSQVTLLLRVCFIILTLTSIGFLLDQRPKAAIMETLRCLMFLMLYRFGHLKPLVPSLSSAFEIVFSICIAFWGVRSMKQLTSHPWK

SEQ ID NO: 3 - DNA, 2475 bp, Homo sapiens
ctctctacacagaatcggctgttgagtgtgctctcagtggagctttggttttagctgttctctgacaaagagcttgttctgagctgcacatctcgtcctctttgttcagcctcaggcttcaagcattgaatcctaaatattctccagctgggaatcagacaagggcagaaatgaagaacccagaagcccagcaggatgtttcagtttcccagggatttcgcatgttgttttacacgatgaaacccagtgaaacttcattccaaacattagaagaggtgcctgattatgtaaaaaaggcaactccatttttcatttctttgatgctgcttgaacttgttgtcagctggattctcaaaggaaagccaccaggtcgcctggatgatgctttaacgtcaatctcagctggtgttctgtctcgacttccaagtctatttttcaggagcattgaactgaccagttatatttatatctgggagaactacaggctgttcaatttgccttgggattctccatggacttggtattcagccttcttaggagttgactttggctactactggttccatcgtatggctcatgaagttaatattatgtgggccggacaccaaacacatcatagttctgaagactataacttatccacagcactgagacagtctgtcctccagatatatacttcctggattttctactctcccctggccctcttcatacccccttcagtatatgctgttcatcttcaattcaatcttctttaccaattttggatccatacagaggtcatcaataaccttggtcctttggaactgattcttaatactcctagccatcatagggttcatcatggcagaaatcgttattgcatagacaaaaattatgctggtgttcttattatttgggataaaatttttgggacatttgaagcagaaaatgaaaaagttgtatatggcttaacacatcccattaatacatttgaaccaatcaaagtgcagttccatcacttattttccatatggactacattctgggccacacctggattcttcaataagttttctgtcatatttaagggaccgggatggggtccaggtaaaccaagacttggtctcagtgaagaaattccagaggtcaccggcaaagaagttcccttctcatcatcttcatctcagctattaaagatatatacagttgtacagtttgctctgatgttggcattttatgaagagacctttgcagatacagctgcactgtcgcaagttactctccttctgagggtttgcttcattatcctgaccttgacttccattggatttcttctggatcaaagacccaaggcagctattatggaaactctccgttgcttgatgttcttaatgctgtaccgatttggtcacctgaagcctcttgtcccttcattgtcatctgcttttgagattgttttttccatttgcattgctttctggggagttagaagcatgaaacaactcacctctcacccttggaaataacctgaatttgtacataattctcttcttttaatgagttgtccacacgcatattatgactgcatattaaaatgtaattattttatgtaatgcttatatgaactatttcttcaatgaaaagtaaaattacttatttactattgtttgcctttcacatttgttattttctattaaaaattaaagtcagttttggttacttcccccctttactacaattaaaaaaagatttcaaatataatgatgttatattaactgatagccttatatgacaagtataaaaagaagggatgaaacttaaaaacagtaaaaacaagaaggaatattgcctttacatcaatttgaaaacaatgtttcctttgatgtttgctaaaattatgcatagatacatgtttgtagtcataaaaatgtattacattggttgtcttcctaaggccacagttacctttgcaatccatataacctaagaagctgcattccagaaaaagacatcactgaggccaggcgcggtggctcacccctgtaatcccagcactttgtggggctgaggtgggcggatcatgaggtccagagatagagaccatcctggccaacatggtgaagtcctgtctctactaaaaatataaaaatttagctggacatggtggtgtgcgcctgtagtcccagctactctggaggctgaggcaggagaatcgcttgaacctgggaggcagaagttggagtgagtggagattgcaccactgcactccagcctgggcgacagagcgagactccgtctcaaagaaaaaagacatcactgaaagaaaaatgaacagaatttgtcagaattagttttttcaacaggttactttgtcatacatttctctaatatgcttggtcaatttgttttggcagactgggcagcatgcagcaattctgcattatttaaagttatcagaacaatgttaattctctaaataaaattacccaaggt